(19) United States
(12) Patent Application Publication
(10) Pub. No.: US 2024/0285755 A1
(43) Pub. Date: Aug. 29, 2024

(54) Adjuvants

(71) Applicant: GlaxoSmithKline Biologicals SA, Rixensart (BE)

(72) Inventor: Emmanuel Jules HANON, Rixensart (BE)

(73) Assignee: GlaxoSmithKline Biologicals SA, Rixensart (BE)

(21) Appl. No.: 18/562,520

(22) PCT Filed: May 20, 2022
(86) PCT No.: PCT/EP2022/063709

§ 371 Date: Nov. 20, 2023

Related U.S. Application Data
(60) Provisional application No. 63/192,241, filed on May 24, 2021.
(60) Provisional application No. 63/232,366, filed on Aug. 12, 2021.

(51) Int. Cl. A61K 39/39; A61K 39/00

(52) U.S. Cl. CPC A61K 39/39; A61K 2039/53; A61K 2039/55561

(57) ABSTRACT

The present invention relates to immunisation using carrier-formulated mRNA in conjunction with an adjuvant comprising a STING agonist, and to related aspects.

### CROSS-REFERENCE TO RELATED APPLICATIONS

This application is a National Stage Application under 35 U.S.C. § 371 of International Application No. PCT/EP2022/063709, filed 20 May 2022, which claims the benefit of U.S. Provisional Application No. 63/192,241, filed 24 May 2021, and U.S. Provisional Application No. 63/232,366, filed 12 Aug. 2021, all of which are incorporated herein by reference in their entireties.

### REFERENCE TO SEQUENCE LISTING SUBMITTED ELECTRONICALLY

The instant application contains a Sequence Listing which has been submitted in ASCII format and is hereby incorporated by reference in its entirety. Said ASCII copy, created on Dec. 1, 2022, is named “24733280_VB67097WO01_Sequence_Listing_as_filed.txt” and is 96,472 bytes in size.

### TECHNICAL FIELD

The present invention relates to carrier-formulated mRNA immunisation in conjunction with an adjuvant comprising a STING agonist, and to related aspects.

### BACKGROUND ART

Messenger RNA (mRNA) is a single-stranded RNA molecule that corresponds to the genetic sequence of a gene and is read by ribosomes in the process of producing a protein. mRNA based vaccines provide an alternative vaccination approach to traditional strategies involving live attenuated/inactivated pathogens or subunit vaccines (Zhang, 2019). mRNA vaccines may utilise non-replicating mRNA or self-replicating RNA (also referred to as self-replicating or self-amplifying mRNA (‘SAM’)). Non-replicating mRNA-based vaccines typically encode an antigen of interest and contain 5′ and 3′ untranslated regions (UTRs), a 5′ cap and a poly(A) tail; whereas self-amplifying RNAs also encode viral replication machinery that enables intracellular RNA amplification (Pardi, 2018).

Adjuvants are sometimes included in vaccines to improve humoral and/or cellular immune responses, particularly in the case of poorly immunogenic subunit vaccines. Similar to natural infections by pathogens, adjuvants rely on the activation of the innate immune system to promote long-lasting adaptive immunity. As simultaneous activation of multiple innate immune pathways is a feature of natural infections, adjuvants may combine multiple immunostimulants in order to promote adaptive immune responses to vaccination.

STING (STimulator of InterferoN Genes) plays an important role in innate immunity. It is an intracellular receptor, part of the pattern-recognition receptor (PRR) family. Upon activation, it triggers the STING signalling pathway (see, e.g., Dubensky, 2013).

There remains a need for the provision of further immunisation approaches that may have benefits such as reduced raw material need and/or reduced unsolicited effects (e.g. reduced reactogenicity), particularly while maintaining an adequate or even improved immune response.

### SUMMARY OF THE INVENTION

The inventors have found that adjuvants comprising a STING agonist may be of benefit in conjunction with carrier-formulated mRNA encoding an antigen.

The invention therefore provides a method of eliciting an immune response in a subject, the method comprising administering to the subject (i) carrier-formulated mRNA wherein the mRNA encodes an antigen, and (ii) an adjuvant comprising a STING agonist. Further provided is a method of adjuvanting the immune response of a subject to an antigen expressed following administration of carrier-formulated mRNA wherein the mRNA encodes an antigen, the method comprising administering to the subject an adjuvant comprising a STING agonist.

The invention also provides an adjuvant comprising a STING agonist for use in eliciting an immune response in a subject by administration with carrier-formulated mRNA wherein the mRNA encodes an antigen. Also provided is carrier-formulated mRNA wherein the mRNA encodes an antigen, for use in eliciting an immune response in a subject by administration with an adjuvant comprising a STING agonist.

The invention also provides the use of an adjuvant comprising a STING agonist in the manufacture of a medicament for use in eliciting an immune response in a subject by administration with carrier-formulated mRNA wherein the mRNA encodes an antigen. Also provided is the use of carrier-formulated mRNA, wherein the mRNA encodes an antigen, in the manufacture of a medicament for use in eliciting an immune response in a subject by administration with an adjuvant comprising a STING agonist.

The invention also provides an immunogenic composition comprising (i) carrier-formulated mRNA wherein the mRNA encodes an antigen, and (ii) an adjuvant comprising a STING agonist. Also provided is a kit comprising: (i) a first container comprising carrier-formulated mRNA wherein the mRNA encodes an antigen; and (ii) a second container comprising an adjuvant comprising a STING agonist. Additionally provided is a kit comprising: (i) a first container comprising carrier-formulated mRNA wherein the mRNA encodes an antigen; (ii) a second container comprising an adjuvant comprising a STING agonist, and (iii) instructions for combining the carrier-formulated mRNA (such as a single dose of the carrier-formulated mRNA) with the adjuvant comprising a STING agonist (such as a single dose of the adjuvant comprising a STING agonist) to produce an immunogenic composition prior to administration of a single dose of the immunogenic composition to a subject.

The invention also provides the use of (i) carrier-formulated mRNA wherein the mRNA encodes an antigen, and (ii) an adjuvant comprising a STING agonist, in the manufacture of a medicament.

### BRIEF DESCRIPTION OF THE SEQUENCES

SEQ ID NO. 1: SARS-CoV-2 S protein
SEQ ID NO. 2: SARS-CoV-2 S protein ectodomain
SEQ ID NO. 3: SARS-CoV-2 S protein receptor binding domain
SEQ ID NO. 4: Pre-fusion stabilised SARS-CoV-2 S protein ectodomain
SEQ ID NO. 5: Polynucleotide sequence of HSV-2 gE/gI insert
SEQ ID NO. 6: Polynucleotide sequence of parent SAM pTC83R_P989 plasmid
SEQ ID NO: 7: Polynucleotide sequence of SAM gE_P317R_IRES_gI transcript
SEQ ID NO: 8: SAM pTC83R_P989 transcript theoretical RNA sequence
SEQ ID NO: 9: Polypeptide sequence of HSV-2 gE P317R
SEQ ID NO: 10: Polypeptide sequence of HSV-2 gI
SEQ ID NO: 11: Polynucleotide sequence of mRNA encoding HSV-2 gE
SEQ ID NO: 12: Polynucleotide sequence of chemically modified mRNA encoding HSV-2 gE
SEQ ID NO: 13: Polypeptide sequence of encoded HSV-2 gE

### BRIEF DESCRIPTION OF THE FIGURES

FIG. 1: Evaluation of total anti-HSV-2 gE IgG antibody response measured in serum samples collected after one (24P1) or two (21PII) 0.1ug doses of LNP/SAM-gE_P317R/gI vaccine co-administered at different timings and with different doses of a STING agonist adjuvant.

FIG. 2: Evaluation of total anti-HSV-2 gI IgG antibody response measured in serum samples collected after one (24P1) or two (21 PII) 0.1 ug doses of LNP/SAM-gE_P317R/gI vaccine co-administered at different timings and with different doses of a STING agonist adjuvant.

FIG. 3: Evaluation of total anti-HSV-1 gE/gI IgG antibody response measured in serum samples collected after one (24P1) or two (21PII) 0.1 ug doses of LNP/SAM-gE_P317R/gI vaccine co-administered at different timings and with different doses of a STING agonist adjuvant.

FIG. 4: Inhibition of hIgG Fc binding activity by HSV-2 gE/gI protein (ED50) 21 days after the second dose of 0.lug LNP/SAM-gE_P317R/gI vaccine co-administered at different timings and with different doses of a STING agonist adjuvant.

FIG. 5: Percentage of anti-HSV-2 gE or gI-specific CD4+ T cell responses induced in CB6F1 mice 21 days after second immunization with 0.lug LNP/SAM-gE_P317R/gI vaccine co-administered at different timings and with different doses of a STING agonist adjuvant.

FIG. 6: Percentage of anti-HSV-2 gE or gI-specific CD8+ T cell responses induced in CB6F1 mice 21 days after second immunization with 0.lug LNP/SAMgE_P317R/gI vaccine co-administered at different timings and with different doses of a STING agonist adjuvant.

FIG. 7: Evaluation of total anti-HSV-2 gE IgG antibody response measured in serum samples collected after one or two 0.8ug doses of LNP/mRNA (native) vaccine co-administered at different timings and with different doses of a STING agonist adjuvant.

FIG. 8: Evaluation of total anti-HSV-2 gE IgG antibody response measured in serum samples collected after one or two 0.8ug doses of LNP/mRNA (N1 m4ψ) vaccine co-administered at different timings and with different doses of a STING agonist adjuvant.

FIG. 9: Evaluation of total anti-HSV-1 gE/gI cross-reactive IgG antibody response measured in serum samples collected after one or two 0.8ug doses of LNP/mRNA (native) vaccine co-administered at different timings and with different doses of STING adjuvant.

FIG. 10: Evaluation of total anti-HSV-1 gE/gI cross-reactive IgG antibody response measured in serum samples collected after one or two 0.8ug doses of LNP/mRNA (N1 m4) vaccine co-administered at different timings and with different doses of a STING agonist adjuvant.

FIG. 11: Inhibition of hIgG Fc binding activity by HSV-2 gE/gI protein (ED50) 14PII for LNP/mRNA (native) vaccine co-administered at different timings and with different doses of a STING agonist adjuvant.

FIG. 12: Inhibition of hIgG Fc binding activity by HSV-2 gE/gI protein (ED50) 14PII for LNP/mRNA (N1 m4) vaccine co-administered at different timings and with different doses of a STING agonist adjuvant.

FIG. 13: Percentage of anti-HSV-2 gE or anti-HSV-1 gE cross-reactive CD4+ T cell responses induced in CB6F1 mice 14 days after second immunization with 0.8ug LNP/mRNA (native) vaccine co-administered at different timings and with different doses of a STING agonist adjuvant.

FIG. 14: Percentage of anti-HSV-2 gE or anti-HSV-1 gE cross-reactive CD4+ T cell responses induced in CB6F1 mice 14 days after second immunization with 0.8ug LNP/mRNA (N1m4J) vaccine co-administered at different timings and with different doses of a STING agonist adjuvant.

FIG. 15: Percentage of anti-HSV-2 gE or anti-HSV-1 gE cross-reactive CD8+ T cell responses induced in CB6F1 mice 14 days after second immunization with 0.8ug LNP/mRNA (native) vaccine co-administered at different timings and with different doses of a STING agonist adjuvant.

FIG. 16: Percentage of anti-HSV-2 gE or anti-HSV-1 gE cross-reactive CD8+ T cell responses induced in CB6F1 mice 14 days after second immunization with 0.8ug LNP/mRNA (N1m4J) vaccine co-administered at different timings and with different doses of a STING agonist adjuvant.

### DETAILED DESCRIPTION OF THE INVENTION

As mentioned previously, the inventors have found that adjuvants comprising a STING agonist may be of benefit in conjunction with carrier-formulated mRNA encoding an antigen. mRNA

Messenger RNA (mRNA) can direct the cellular machinery of a subject to produce proteins. mRNA may be circular or branched, but will generally be linear.

mRNA used herein are preferably provided in purified or substantially purified form i.e. substantially free from proteins (e.g., enzymes), other nucleic acids (e.g. DNA and nucleoside phosphate monomers), and the like, generally being at least about 50% pure (by weight), and usually at least 90% pure, such as at least 95% or at least 98% pure.

mRNA may be prepared in many ways e.g. by chemical synthesis in whole or in part, by digesting longer nucleic acids using nucleases (e.g. restriction enzymes), by joining shorter nucleic acids or nucleotides (e.g. using ligases or polymerases), from genomic or cDNA libraries, etc. In particular, mRNA may be prepared enzymatically using a DNA template.

The term mRNA as used herein includes conventional mRNA (also called unmodified or native mRNA) or mRNA analogs, such as those containing modified backbones or modified bases (e.g. pseudouridine, or the like). mRNA, may or may not have a 5′ cap.

The mRNA comprises a sequence which encodes at least one antigen.

Typically, the nucleic acids of the invention will be in recombinant form, i.e. a form which does not occur in nature. For example, the mRNA may comprise one or more heterologous nucleic acid sequences (e.g. a sequence encoding another antigen and/or a control sequence such as a promoter or an internal ribosome entry site) in addition to the sequence encoding the antigen.

Alternatively, or in addition, the sequence or chemical structure of the nucleic acid may be modified compared to a naturally-occurring sequence which encodes the antigen. The sequence of the nucleic acid molecule may be modified, e.g. to increase the efficacy of expression or replication of the nucleic acid, or to provide additional stability or resistance to degradation.

mRNA may also be codon optimised. In some embodiments, mRNA may be codon optimised for expression in human cells. By “codon optimised” is intended modification with respect to codon usage which may increase translation efficacy and/or half-life of the nucleic acid.

A poly A tail (e.g., of about 30 adenosine residues or more) may be attached to the 3′ end of the RNA to increase its half-life.

The 5′ end of the RNA may be capped, for example with a modified ribonucleotide with the structure m7G (5) ppp (5) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methyltransferase, which catalyzes the construction of N7-monomethylated cap 0 structures). Cap 0 structure plays an important role in maintaining the stability and translational efficacy of the mRNA molecule. The 5′ cap of the mRNA molecule may be further modified by a 2′-O-Methyltransferase which results in the generation of a cap 1 structure (m7Gppp [m2′-O] N), which may further increase translation efficacy.

mRNA may comprise one or more nucleotide analogs or modified nucleotides. As used herein, “nucleotide analog” or “modified nucleotide” refers to a nucleotide that contains one or more chemical modifications (e.g., substitutions) in or on the nitrogenous base of the nucleoside (e.g. cytosine (C), thymine (T) or uracil (U)), adenine (A) or guanine (G)). A nucleotide analog can contain further chemical modifications in or on the sugar moiety of the nucleoside (e.g. ribose, modified ribose, six-membered sugar analog, or open-chain sugar analog), or the phosphate. The preparation of nucleotides and modified nucleotides and nucleosides are well-known in the art, see the following references: U.S. Pat. Nos. 4,373,071, 4,458,066, 4,500,707, 4,668,777, 4,973,679, 5,047,524, 5,132,418, 5,153,319, 5,262,530, 5,700,642. Many modified nucleosides and modified nucleotides are commercially available. Modified nucleobases (chemical modifications) which can be incorporated into modified nucleosides and nucleotides and be present in the mRNA molecules include: m5C (5-methylcytidine); m5U (5-methyluridine); m6A (N6-methyladenosine); s2U (2-thiouridine); Um (2′-O-methyluridine); m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); m′I (1-methylinosine); m′Im (I,2′-O-dimethylinosine); m3C (3-methylcytidine); Cm (2′-O-methylcytidine); s2C (2-thiocytidine); ac4C (N4-acetylcytidine); f5C (5-fonnylcytidine); m5Cm (5,2-O-dimethylcytidine); ac4Cm (N4-acetyl-2-O-methylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-O-methyl uridine); cmnm5s2U (5-carboxymethylaminomethyl-2-thiouridine); m62A (N6,N6-dimethyladenosine); Im (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,2′-O-dimethyladenosine); m62Am (N6,N6,0-2-trimethyladenosine); m2′7G (N2,7-dimethylguanosine); m2′2′7G (N2,N2,7-trimethylguanosine); m3Um (3,2′-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C₂-C₆)-alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides are available from commercial suppliers.

The mRNA may encode more than one antigen. For example, the mRNA encoding an antigen protein may encode only the antigen or may encode additional proteins. Each antigen and additional protein(s) may be under the control of different regulatory elements. Alternatively, the antigen and additional proteins may be under the control of the same regulatory element. Where at least two additional proteins are encoded, some if the antigen and additional proteins may be under the control of the same regulatory element and may be under the control of different regulatory elements.

mRNA may be non-replicating or may be replicating, also known as self-replicating. A self-replicating mRNA molecule may be an alphavirus-derived mRNA replicon. mRNA amplification can also be achieved by the provision of a non-replicating mRNA encoding an antigen in conjunction with a separate mRNA encoding replication machinery.

Self-replicating mRNA molecules are well known in the art and can be produced by using replication elements derived from, e.g., alphaviruses, and substituting the structural viral proteins with a nucleotide sequence encoding a protein of interest. A self-replicating mRNA molecule is typically a +-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells.

Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons, see the following reference: WO2005/113782.

In certain embodiments, the self-replicating mRNA molecule described herein encodes (i) an RNA-dependent RNA polymerase which can transcribe RNA from the self-replicating mRNA molecule and (ii) an antigen. The polymerase can be an alphavirus replicase e.g. comprising one or more of alphavirus proteins nsPI, nsP2, nsP3 and nsP4.

Whereas natural alphavirus genomes encode structural virion proteins in addition to the non-structural replicase polyprotein, in certain embodiments, the self-replicating mRNA molecules do not encode alphavirus structural proteins. Thus, the self-replicating mRNA can lead to the production of genomic RNA copies of itself in a cell, but not to the production of RNA-containing virions. The inability to produce these virions means that, unlike a wild-type alphavirus, the self-replicating mRNA molecule cannot perpetuate itself in infectious form. The alphavirus structural proteins which are necessary for perpetuation in wild-type viruses are absent from self-replicating mRNAs of the present disclosure and their place is taken by gene(s) encoding the immunogen of interest, such that the subgenomic transcript encodes the immunogen rather than the structural alphavirus virion proteins.

Thus, a self-replicating mRNA molecule useful with the invention may have two open reading frames. The first (5) open reading frame encodes a replicase; the second (3′) open reading frame encodes an antigen. In some embodiments the self-replicating mRNA may have additional (e.g. downstream) open reading frames e.g. to encode further antigens or to encode accessory polypeptides.

In certain embodiments, the self-replicating mRNA molecule disclosed herein has a 5′ cap (e.g. a 7-methylguanosine). This cap can enhance in vivo translation of the RNA. In some embodiments, the 5′ sequence of the self-replicating mRNA molecule must be selected to ensure compatibility with the encoded replicase.

A self-replicating mRNA molecule may have a 3′ poly-A tail. It may also include a poly-A polymerase recognition sequence (e.g. AAUAAA) near its 3′ end.

Self-replicating mRNA molecules can have various lengths, but they are typically 5000 to 25000 nucleotides long, such as 8000 to 15000 nucleotides long, for example 9000 to 12000 nucleotides long. Self-replicating mRNA molecules will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

In another embodiment, a self-replicating mRNA may comprise two separate mRNA molecules, each comprising a nucleotide sequence derived from an alphavirus: one RNA molecule comprises an RNA construct for expressing alphavirus replicase, and one RNA molecule comprises an RNA replicon that can be replicated by the replicase in trans. In some embodiments, the RNA construct for expressing alphavirus replicase comprises a 5′-cap. See WO2017/162265.

The self-replicating mRNA can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-replicating mRNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-replicating mRNA can include (in addition to any 5′ cap structure) one or more nucleotides having a modified nucleobase. An RNA used with the invention ideally includes only phosphodiester linkages between nucleosides, but in some embodiments it can contain phosphoramidate, and/or methylphosphonate linkages.

The self-replicating mRNA molecule may encode a single heterologous polypeptide antigen (i.e. the antigen) or, optionally, two or more heterologous polypeptide antigens linked together in a way that each of the sequences retains its identity (e.g., linked in series) when expressed as an amino acid sequence. The heterologous polypeptides generated from the self-replicating mRNA may then be produced as a fusion polypeptide or engineered in such a manner to result in separate polypeptide or peptide sequences.

The self-replicating mRNA molecules described herein may be engineered to express multiple nucleotide sequences, from two or more open reading frames, thereby allowing co-expression of proteins, such as one, two or more antigens (e.g. one, two or more coronavirus protein(s), such as SARS-CoV-2 S protein(s)) together with cytokines or other immunomodulators, which can enhance the generation of an immune response. Such a self-replicating mRNA molecule might be particularly useful, for example, in the production of various gene products (e.g., proteins) at the same time, for example, as a bivalent or multivalent vaccine.

If desired, the self-replicating mRNA molecules can be screened or analyzed to confirm their therapeutic and prophylactic properties using various in vitro or in vivo testing methods that are known to those of skill in the art. For example, vaccines comprising self-replicating mRNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self-replicating mRNA molecule that encodes an antigen. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry.

Self-replicating mRNA molecules that encode an antigen can also be tested for ability to induce humoral immune responses, as evidenced, for example, by induction of B cell production of antibodies specific for the antigen of interest. These assays can be conducted using, for example, peripheral B lymphocytes from immunized individuals. Such assay methods are known to those of skill in the art. Other assays that can be used to characterize the self-replicating mRNA molecules can involve detecting expression of the encoded antigen by the target cells. For example, FACS can be used to detect antigen expression on the cell surface or intracellularly. Another advantage of FACS selection is that one can sort for different levels of expression; sometimes-lower expression may be desired. Other suitable method for identifying cells which express a particular antigen involve panning using monoclonal antibodies on a plate or capture using magnetic beads coated with monoclonal antibodies.

A non-replicating mRNA will typically contain 10000 bases or fewer, especially 8000 bases or fewer, in particular 5000 bases or fewer. A replicating mRNA will typically contain 25000 bases or fewer, especially 20000 bases or fewer, in particular 15000 bases or fewer. A replicating mRNA may contain 5000 to 25000 nucleotides, such as 8000 to 15000 nucleotides, for example 9000 to 12000 nucleotides.

A single dose of mRNA may be 0.001 to 1000 ug, especially 1 to 500 ug, in particular 10 to 250 ug. A single dose of mRNA may be 0.001 to 75 ug, 1 to 75 ug, 25 to 250 ug, or 250 to 1000 ug. Specifically, a replicating mRNA dose may be 0.001 to 75 ug, such as 0.1 to 75 ug. A non-replicating mRNA dose may, for example, be 1 to 500 ug, such as 1 to 250 ug.

In one embodiment the mRNA is non-replicating mRNA. In a second embodiment the mRNA is replicating mRNA.

mRNA Carriers
A range of carrier systems have been described which encapsulate or complex mRNA in order to facilitate mRNA delivery and consequent expression of encoded antigens as compared to mRNA which is not encapsulated or complexed. The present invention may utilise any suitable carrier system. Particular mRNA carrier systems of note are further described below.

### LNP

Lipid nanoparticles (LNPs) are non-virion liposome particles in which mRNA can be encapsulated. LNP delivery systems and methods for their preparation are known in the art. The particles can include some external mRNA (e.g. on the surface of the particles), but desirably at least half of the RNA (and suitably at least 85%, especially at least 95%, such as all of it) is encapsulated.

LNP formulated mRNA may be prepared comprising mRNA, cationic lipid, and other helper lipids. Liposomal particles can, for example, be formed of a mixture of zwitterionic, cationic and anionic lipids which can be saturated or unsaturated, for example; DSPC (zwitterionic, saturated), DlinDMA (cationic, unsaturated), and/or DMG (anionic, saturated). Preferred LNPs for use with the invention include an amphiphilic lipid which can form liposomes, optionally in combination with at least one cationic lipid (such as DOTAP, DSDMA, DODMA, DLinDMA, DLenDMA, etc.). A mixture of DSPC, DlinDMA, PEG-DMG and cholesterol is particularly effective. Other useful LNPs are described in the following references: Include WO 2012006372, WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9.

LNP can, for example, be formed of a mixture of (i) a PEG-modified lipid (ii) a non-cationic lipid (iii) a sterol (iv) an ionisable cationic lipid. Alternatively, LNP can for example be formed of a mixture of (i) a PEG-modified lipid (ii) a non-cationic lipid (iii) a sterol (iv) a non-ionisable cationic lipid.

The PEG-modified lipid may comprise a PEG molecule with a molecular weight of 10000 Da or less, especially 5000 Da or less, in particular 3000 Da, such 2000 Da or less. Examples of PEG-modified lipids include PEG-distearoyl glycerol, PEG-dipalmitoyl glycerol and PEG-dimyristoyl glycerol. The PEG-modified lipid is typically present at around 0.5 to 15 molar %.

The non-cationic lipid may be a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) and sphingomyelin (SM). The non-cationic lipid is typically present at around 5 to 25 molar %.

The sterol may be cholesterol. The sterol is typically present at around 25 to 55 molar A range of suitable ionizable cationic lipids are known in the art, which are typically present at around 20 to 60 molar %.

The ratio of RNA to lipid can be varied (see for example WO2013/006825). “N:P ratio” refers to the molar ratio of protonatable nitrogen atoms in the cationic lipids (typically solely in the lipid's headgroup) to phosphates in the RNA. The ratio of nucleotide (N) to phospholipid (P) can be in the range of, e.g., 1N:1P to 20N:1P, 1N:1P to 10N:1P, 2N:1P to 8N:1P, 2N:1P to 6N:1P or 3N:1P to 5N:1P. The ratio of nucleotide (N) to phospholipid (P) can be in the range of, e.g., 1N:1P, 2N:1P, 3N:1P, 4N:1P, 5N:1P, 6N:1P, 7N:1P, 8N:1P, 9N:1P, or 10N:1P. Alternatively or additionally, the ratio of nucleotide (N) to phospholipid (P) is 4N:1P.

WO2017/070620 provides general information on LNP compositions and is incorporated herein by reference. Other useful LNPs are described in the following references: WO2012/006376 (LNP and microparticle delivery systems); WO2012/006359 (microparticle delivery systems); WO2012/030901; WO2012/031046; WO2012/031043; WO2012/006378; WO2011/076807; WO2013/033563; WO2013/006825; WO2014/136086; WO2015/095340; WO2015/095346; WO2016/037053, Geall et al. (2012) PNAS USA. September 4; 109(36): 14604-9 (LNP delivery system), which are also incorporated herein by reference.

LNPs are typically 50 to 200 um in diameter (Z-average). Suitably the LNPs have a polydispersity of 0.4 or less, such as 0.3 or less.

In one embodiment the carrier is a lipid nanoparticle (LNP).

### CNE

The carrier may be a cationic nanoemulsion (CNE) delivery system. Such cationic oil-in-water emulsions can be used to deliver the mRNA to the interior of a cell. The emulsion particles comprise a hydrophobic oil core and a cationic lipid, the latter of which can interact with the mRNA, thereby anchoring it to the emulsion particle. In a CNE delivery system, the mRNA which encodes the antigen is complexed with a particle of a cationic oil-in-water emulsion. CNE carriers and methods for their preparation are described in WO2012/006380, WO2013/006837 and WO2013/006834 which are incorporated herein by reference.

Thus, the mRNA may be complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. Alternatively or additionally, the CNE comprises squalene and a cationic lipid, such as 1,2-dioleoyloxy-3-(trimethylammonium)propane (DOTAP) (see e.g. Brito, 2014). In an embodiment, the CNE is an oil-in-water emulsion of DOTAP and squalene stabilised with polysorbate 80 and/or sorbitan trioleate.

Desirably at least half of the RNA (and suitably at least 85%, such as all of it) is complexed with the cationic oil-in-water emulsion carrier.

CNE are typically 50 to 200 um in diameter (Z-average). Suitably the CNE have a polydispersity of 0.4 or less, such as 0.3 or less.

In one embodiment the carrier is a cationic nanoemulsion (CNE).

### LION

A lipidoid-coated iron oxide nanoparticle (LION) is capable of delivering mRNA into cells and may be aided after administration to a subject by application of an external magnetic field. A LION is an iron oxide particle with one or more coatings comprising lipids and/or lipidoids wherein mRNA encoding the antigen is incorporated into or associated with the lipid and/or lipidoid coating(s) through electrostatic interactions. The mRNA being embedded within the coating(s) may offer protection from enzymatic degradation. The lipids and/or lipidoids comprised within a LION may for example include those included in Figure S1 of Jiang, 2013, especially lipidoids comprising alkyl tails of 12 to 14 carbons in length and in particular lipidoid C14-200 as disclosed in Jiang, 2013. A LION may typically comprise 200 to 5000, such as 500 to 2000, in particular about 1000 lipid and/or lipidoid molecules. Typically the LIONs are 20 to 200 nm in diameter, especially 50 to 100 nm in diameter. The lipid/lipidoid to mRNA weight ratio may be about 1:1 to 10:1, especially about 5:1. Particularly suitable LIONs, and methods for preparation of LIONs are disclosed in Jiang, 2013.

In one embodiment the carrier is a lipidoid-coated iron oxide nanoparticle (LION).

### Antigen

According to the present invention, the adjuvant comprising a STING agonist is to be utilised in conjunction with carrier-formulated mRNA wherein the mRNA encodes an antigen (also referred to herein simply as ‘carrier-formulated mRNA’). By the term carrier-formulated mRNA’ is meant mRNA that is formulated with a carrier to facilitate delivery and/or improved stability. Suitable carriers include LNP, CNE and LION.

By the term antigen is meant a polypeptide which is capable of eliciting an immune response in a subject. Suitably the immune response is a protective immune response, e.g. reducing partially or completely the severity of one or more symptoms and/or time over which one or more symptoms are experienced by a subject, reducing the likelihood of developing an established infection after challenge and/or slowing progression of an associated illness (e.g. extending survival).

Suitably the antigen comprises at least one B or T cell epitope, suitably an antigen comprises B and T cell epitopes. The elicited immune response may be an antigen specific B cell response which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

Suitably the encoded antigen contains 3000 residues or fewer, especially 2000 residues or fewer, in particular 1500 residues or fewer. The encoded antigen may contain 1000 residues or fewer, 800 residues or fewer, 600 residues or fewer, 400 residues or fewer or 200 residues or fewer.

Suitably the antigen contains 50 residues or more, especially 100 residues or more, in particular 150 residues or more.

Suitably the antigen contains 50 to 3000 residues, especially 100 to 1500 residues, in particular 200 to 1000 residues.

The antigen may be derived from a pathogen, especially a human pathogen, (such as a bacterium, virus or parasite) or may be a cancer antigen (such as a tumour antigen and/or a neoantigen).

In one embodiment, the antigen is derived from a coronavirus, particularly from SARS-CoV-2.

A plurality of antigens may be encoded. Consequently, in some embodiments the antigen is derived from at least one coronavirus, for example from SARS-CoV-2. In some embodiments the antigen is derived from more than one coronavirus (such as 2, 3, 4 or 5), for example from SARS-CoV-2 (such as a plurality of SARS-CoV-2 variant antigens).

SARS-CoV-2 makes use of a densely glycosylated spike (S) protein to gain entry into host cells. In coronaviruses, the S protein is a trimeric class I fusion protein which exists in a metastable pre-fusion conformation that undergoes a substantial structural rearrangement to fuse the viral membrane with the host cell membrane (Li, 2016; Bosch, 2003).

A coronavirus protein of use in the present invention is a fragment or variant of a native coronavirus protein which is capable of eliciting neutralising antibodies and/or a T cell response (such as a CD4 or CD8 T cell response) to a coronavirus, suitably a protective immune response.

A SARS-CoV-2 S protein of use in the present invention comprises, such as consists of, a fragment or variant of a native SARS-CoV-2 S protein which is capable of eliciting neutralising antibodies and/or a T cell response (such as a CD4 or CD8 T cell response) to SARS-CoV-2, suitably a protective immune response.

The encoded SARS-CoV-2 S protein may comprise, such as consist of, a full length S protein (such as SEQ ID NO:1). Alternatively, the encoded SARS-CoV-2 S protein may comprise, such as consist of, an amino acid sequence having at least 90% identity to the amino acid sequence set forth in SEQ ID NO:1. The encoded SARS-CoV-2 S protein may comprise, such as consist of, an amino acid sequence having at least 95% identity to the amino acid sequence set forth in SEQ ID NO:1, especially at least 98% identity to the amino acid sequence set forth in SEQ ID NO:1, in particular at least 99% identity to the amino acid sequence set forth in SEQ ID NO:1, such as 100% identity to the amino acid sequence set forth in SEQ ID NO:1.

The encoded SARS-CoV-2 S protein may comprise, or consist of, one or more domains of a full length SARS-CoV-2 S protein, such as the ectodomain (SEQ ID NO:2) or receptor binding domain (RBD, SEQ ID NO:3), or variants thereof.

The encoded SARS-CoV-2 S protein may comprise, such as consist of, an amino acid sequence having at least 90% identity to the amino acid sequence set forth in SEQ ID NO:2. The encoded SARS-CoV-2 S protein may comprise, such as consist of, an amino acid sequence having at least 95% identity to the amino acid sequence set forth in SEQ ID NO:2, especially at least 98% identity to the amino acid sequence set forth in SEQ ID NO:2, in particular at least 99% identity to the amino acid sequence set forth in SEQ ID NO:2, such as 100% identity to the amino acid sequence set forth in SEQ ID NO:2.

The encoded SARS-CoV-2 S protein may comprise, such as consist of, an amino acid sequence having at least 90% identity to the amino acid sequence set forth in SEQ ID NO:3. The encoded SARS-CoV-2 S protein may comprise, such as consist of, an amino acid sequence having at least 95% identity to the amino acid sequence set forth in SEQ ID NO:3, especially at least 98% identity to the amino acid sequence set forth in SEQ ID NO:3, in particular at least 99% identity to the amino acid sequence set forth in SEQ ID NO:3, such as 100% identity to the amino acid sequence set forth in SEQ ID NO:3.

Suitably the encoded SARS-CoV-2 S protein is pre-fusion stabilised to facilitate appropriate presentation to the immune system. For example, Wrapp and colleagues (Wrapp et al., 2020) produced a recombinant prefusion S ectodomain using a stabilization strategy that proved effective for other betacoronavirus S proteins (Pallesen et al, 2017; Kirchdoerfer et al, 2018). To this end, starting with the SARS-CoV-2 polynucleotide sequence (GenBank accession number MN908947.3), a gene encoding residues 1 to 1208 of SARS-CoV-2 S protein (UniProt accession number PODTC2 version 1 dated 22 Apr. 2020) with proline substitutions at residues 986 and 987, a “GSAS” substitution at the furin cleavage site (residues 682-685) a C-terminal T4 fibritin trimerization motif, an HRV3C protease cleavage site, a TwinStrepTag and an 8×HisTag was synthesized and cloned into the mammalian expression vector pαH.

Residues 1 to 1208 of SARS-CoV-2 S protein with proline substitutions at residues 986 and 987, a “GSAS” substitution at the furin cleavage site are provided in SEQ ID NO:4, which is an example of a pre-fusion stabilized ectodomain of SARS-CoV-2 S protein.

The encoded SARS-CoV-2 S protein may comprise, such as consist of, an amino acid sequence having at least 90% identity to the amino acid sequence set forth in SEQ ID NO:4. The encoded SARS-CoV-2 S protein may comprise, such as consist of, an amino acid sequence having at least 95% identity to the amino acid sequence set forth in SEQ ID NO:4, especially at least 98% identity to the amino acid sequence set forth in SEQ ID NO:4, in particular at least 99% identity to the amino acid sequence set forth in SEQ ID NO:4, such as 100% identity to the amino acid sequence set forth in SEQ ID NO:4.

Suitably the SARS-CoV-2 S protein is a pre-fusion stabilised protein.

In one embodiment, the SARS-CoV-2 S protein is the stabilized recombinant prefusion S ectodomain disclosed by Wrapp et al., 2020.

The SARS-CoV-2 S protein (such as a pre-fusion stabilized SARS-CoV-2 S protein) may desirably be in the form of a trimer and consequently may comprise a trimerization motif, such as a T4 fibritin trimerization motif, more suitably a C-terminal T4 fibritin trimerization motif. Alternative trimerization motifs include, for example, a domain derived from collagen called ‘Trimer-Tag’ such as disclosed in Liu et al., 2017, or a molecular clamp, such as that disclosed in WO2018/176103.

An encoded SARS-CoV-2 S protein is desirably 1800 residues or fewer in length, especially 1500 residues or fewer, in particular 1400 residues or fewer, such as 1300 residues or fewer.

An encoded SARS-CoV-2 S protein is desirably 150 residues or more in length, especially 200 residues or more, in particular 400 residues or more, such as 600 residues or more.

In one embodiment the antigen is a human cytomegalovirus (CMV) antigen.

In one embodiment the antigen is a Zika virus antigen.

In one embodiment the antigen is a human parainfluenza virus (PIV) antigen, such as a human PIV type 3 antigen.

In one embodiment the antigen is a human metapneumovirus (hMPV) antigen.

In one embodiment the antigen is a respiratory syncytial virus (RSV) antigen.

In one embodiment the antigen is an influenza virus antigen, such as a hemagglutinin or a neuraminidase.

In one embodiment the antigen is an Epstein-Barr virus (EBV) antigen.

In one embodiment the antigen is a Herpes simplex virus (HSV) antigen, such as a gE and/or a gI antigen. The gE antigen may be a sequence comprising, such as consisting of, a sequence having at least 80%, such as at least 90%, especially at least 95%, in particular at least 98% for example at least 99% or 100% identity to SEQ ID No: 9. The gI antigen may be a sequence comprising, such as consisting of, a sequence having at least 80%, such as at least 90%, especially at least 95%, in particular at least 98% for example at least 99% or 100% identity to SEQ ID No: 10.

### Sequence Alignments

Identity or homology with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)]can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences.

### Additional Antigens

The present invention may involve a plurality of antigenic components, for example with the objective to elicit a broad immune response e.g. to a pathogen or to elicit responses to multiple pathogens. Consequently, more than one antigen may be present, more than one polynucleotide encoding an antigen may be present, one polynucleotide encoding more than one antigen may be present or a mixture of antigen(s) and polynucleotide(s) encoding antigen(s) may be present. Polysaccharides such as polysaccharide conjugates, may also be present.

### STING Agonists

The STING agonist may be any appropriate agonist that is capable of binding to and activating STING receptor and STING signalling.

In one embodiment, the STING agonist binds to STING with an in vitro K_{f} of less than about 0.750 μM (e.g., less than about 0.500 μM, less than 0.250 μM or less than 0.100 μM).

In one embodiment, the STING agonist activates STING with an in vitro EC₅₀ of about 100 μM or less (e.g., about 50 μM or less, about 20 μM or less, or about 10 μM or less) when measured by monitoring phosphorylation of interferon regulatory factor-3 (IRF3).

In one embodiment, the STING agonist activates STING with an in vitro EC₅₀ of about 100 μM or less (e.g., about 50 μM or less, about 20 μM or less, or about 10 μM or less) as measured by monitoring interferon-p induction.

In one embodiment, the STING agonist is a nucleic acid, a protein, a peptide, or a small molecule. In one embodiment, the STING agonist is a small molecule selected from a modified or unmodified cyclic dinucleotide. In one embodiment, the cyclic dinucleotide is selected from a compound of Formulae (I)-(III):

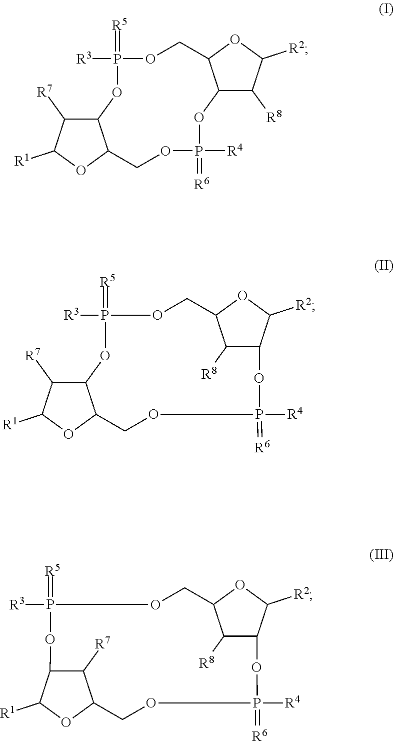

wherein
R¹ and R² are each independently selected from the following groups:

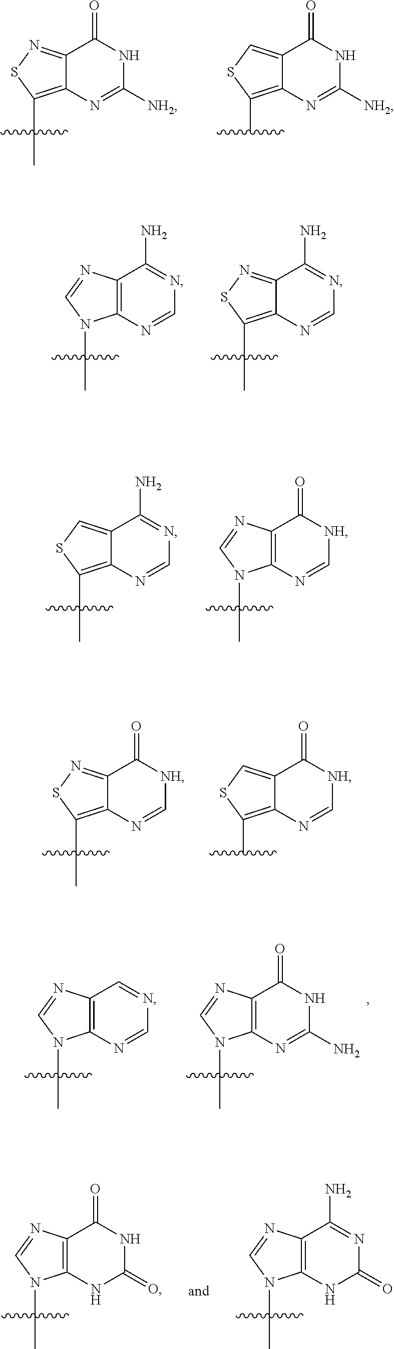

R³ and R⁴ are each independently —SH or —OH,
R⁵ and R⁶ are oxygen or sulphur,
R⁷ and R⁸ are each independently halogen, hydrogen, —OH, or OCH₃.

In one embodiment, the STING agonist is the compound:

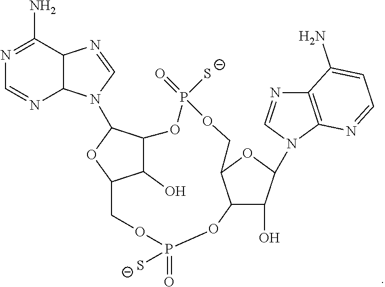 [SMILES: NC1=NC=NC2C1N=CN2C1OC2COP(=O)([S-])OC3C(COP(=O)([S-])OC1C2O)OC(n1cnc2c(N)ccnc21)C3O]

In one embodiment, the cyclic dinucleotide is selected from c-di^{tz}GMP. c-di-^{th}GMP, c-G^{tz}GMP, c-GAMP, c-^{th}GMP, c-^{tz}GMP, c-di-^{th}AMP, c-di^{tz}AMP, c-di-AMP, c-di-GMP, c-diXGMP, c-G^{th}XMP, c-GXMP, c-ACMP, c-A^{th}XMP, c-A^{tz}XMP, c-di-^{th}XMP, or c-di^{tz}XMP (as defined in paragraph [0053] of US 2021/0101924).

In one embodiment, the STING agonist is 2′,3′-cGAMP. In one embodiment, the STING agonist is 3′,3′-cGAMP.

In one embodiment, the STING agonist is the compound:

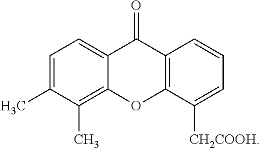

In one embodiment, the STING agonist is the compound 6-bromo-N-(naphthalen-1-yl)benzo[d][1,3]dioxole-5-carboxamide:

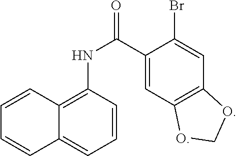 [SMILES: O=C(Nc1cccc2ccccc12)c1cc2c(cc1Br)OCO2]

In one embodiment, the STING agonist is a compound of Formula A:

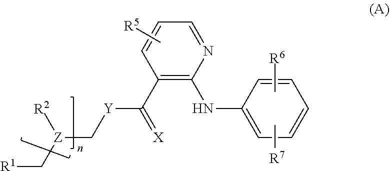

wherein
X is O or NR^{4A}
Y is O, NR^{4A}, CH₂, or absent,
n is 0, 1, 2, or 3,
R¹ and R² are independently selected from OH, OR³, OR^{3A}, SR³, and NR³R⁴,
R³, R⁴, and R^{4A} are independently selected from hydrogen, C1-C10 alkyl optionally substituted with 1-6 halogen, C6-C10 aryl or 5-10 membered heteroaryl, or R³ and R⁴ together with the nitrogen atom to which they are attached form a 3 to 7 membered heterocycle or 5 to 10 membered heteroaryl,
R^{3A} is

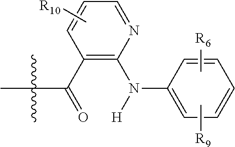

wherein [symbol] represents the point of connection of R^{3A} to the remainder of the molecule,
R⁵-R¹⁰ are independently selected from hydrogen, halogen, pseudohalogen,
C1-C10 alkyl optionally substituted with 1-6 halogens, C6-C10 aryl, and 5 to 10 membered heteroaryl.

In one embodiment, the STING agonist is selected from:

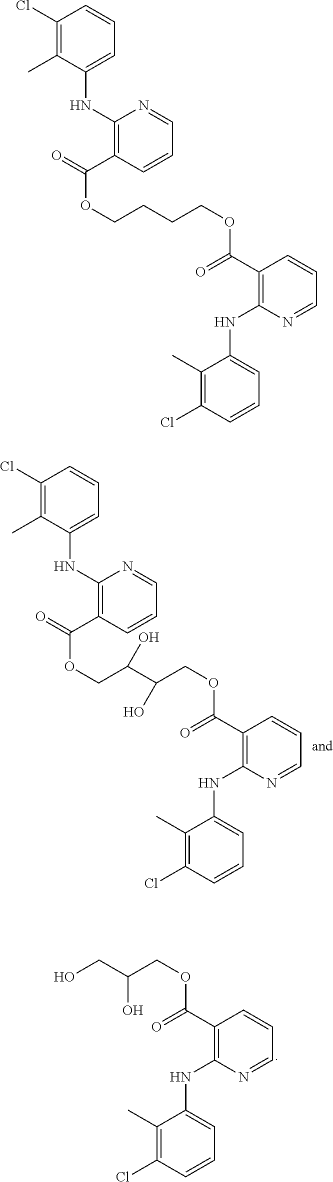 [SMILES: Cc1c(Cl)cccc1Nc1ncccc1C(=O)OCCCCOC(=O)c1cccnc1Nc1cccc(Cl)c1C ; Cc1c(Cl)cccc1Nc1ncccc1C(=O)OCC(O)C(O)COC(=O)c1cccnc1Nc1cccc(Cl)c1C ; Cc1c(Cl)cccc1Nc1ncccc1C(=O)OCC(O)CO]

In one embodiment, the STING agonist is a flavonoid. Suitable flavonoids include, but are not limited to, 10-(carboxymethyl)-9(10H)acridone (CMA), 5,6-Dimethylxanthenone-4-acetic acid (DMXAA), methoxyvone, 6,4′-dimethoxyflavone, 4′-methoxyflavone, 3′,6′-dihydroxyflavone, 7,2′-dihydroxyflavone, daidzein, formononetin, retusin 7-methyl ether, xanthone, or any combination thereof. In one aspect, the STING agonist can be 10-(carboxymethyl)-9(10H)acridone (CMA). In one aspect, the STING agonist can be 5,6-Dimethylxanthenone-4-acetic acid (DMXAA). In one aspect, the STING agonist can be methoxyvone. In one aspect, the STING agonist can be 6,4′-dimethoxyflavone. In one aspect, the STING agonist can be 4′-methoxyflavone. In one aspect, the STING agonist can be 3′,6′-dihydroxyflavone. In one aspect, the STING agonist can be 7,2′-dihydroxyflavone. In one aspect, the STING agonist can be daidzein. In one aspect, the STING agonist can be formononetin. In one aspect, the STING agonist can be retusin 7-methyl ether. In one aspect, the STING agonist can be xanthone. In one aspect, the STING agonist can be any combination of the above flavonoids.

In one embodiment, the STING agonist is a compound of Formula B:

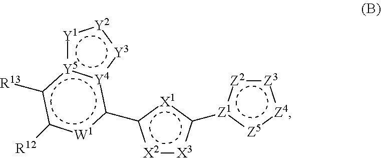

wherein
[symbol] represents two conjugated double bonds in a five-membered heteroaromatic ring and three conjugated double bonds in a six-membered aromatic or heteroaromatic ring,
W¹ is selected from CR¹¹ and N;
X¹ is selected from CR¹, C(R¹)₂, N, NR¹, O and S;
X² is selected from CR², C(R²)₂, N, NR², O and S;
X³ is selected from CR³, C(R³)₂, N, NR³, O and S;
where two or three of X¹, X² and X³ are independently selected from N, NR¹, NR², NR³, O and S; and
where at least one of X¹, X² and X³ is selected from N, NR¹, NR² and NR³;
Y¹ is selected from N, NR⁴, O, S, CR⁴ and C(R⁴)₂;
Y² is selected from N, NR⁵, O, S, CR⁵ and C(R⁵)₂;
Y³ is selected from N, NR⁶, O, S, CR⁶ and C(R⁶)₂;
Y⁴ is selected from C and N;
Y⁵ is selected from C and N;
where at least one and not more than two of Y¹, Y² and Y³ are independently selected from N, NR⁴, NR⁵ and NR⁶;
where when if one of Y⁴ or Y⁵ is N, the other one of Y⁴ or Y⁵ is C;
Z¹ is selected from C and N;
Z² is selected from N, NR⁸ and CR⁸;
Z³ is selected from N, NR⁹ and CR⁹;
Z⁴ is selected from N, NR¹⁰ and CR¹⁰;
Z⁵ is selected from N, NR⁷ and CR⁷;
where two or three of Z¹, Z², Z³, Z⁴ and Z⁵ are independently selected from N, NR⁷, NR⁸, NR⁹, and NR¹⁰;
each R¹ is independently selected from the group consisting of H, C₁-C₈ alkyl, C₁-C₈ alkylene-NRR and C₁-C₈ alkylene-C(O)OR;
each R² is independently selected from the group consisting of H, C₁-C₈ alkyl, C₁-C₈ alkylene-NRR, C₁-C₈ alkylene-C(O)OR, C₁-C₈ alkylene-OR and C₁-C₈ alkylene-O—P(O)(OH)₂;
each R₃ is independently selected from the group consisting of H, C₁-C₈ alkyl, C₁-C₈ alkylene-NRR, C₁-C₈ alkylene-C(O)OR and C₁-C₈ alkylene-O—P(O)(OH)₂; each R⁴ is independently selected from the group consisting of H, —OR, —NRR, C₁-C₈ alkyl optionally substituted with one or two —OR, C₁-C₈ alkylene-NRR, —C(O)OR, C₁-C₈ alkylene-C(O)OR, 3-10 membered heterocycle, C₁-C₈ alkylene-3-10 membered heterocycle optionally substituted with one 3-10 membered heterocycle, (C₃-C₁₀)-cycloalkyl, and C₁-C₈ alkylene-(C₃-C₁₀)-cycloalkyl; each R⁵ is independently selected from the group consisting of H, OR, C₁-C₈ alkyl, —NRR, C₁-C₈ alkylene-NRR, —C(O)OR, C₁-C₈ alkylene-C(O)OR, 3-10 membered heterocycle, C₁-C₈ alkylene-3-10 membered heterocycle optionally substituted with one 3-10 membered heterocycle, and C₁-C₈ alkylene-OR;
each R⁶ is H;
R⁷ is selected from the group consisting of H, halo, hydroxy or NH₂;
R⁸ is selected from the group consisting of H, C₁-C₈ alkyl optionally substituted with one or two —NRR or —OR, C₁-C₈ alkylene-C(O)OR and C₁-C₈ alkylene-SO₂R;
R⁹ is H;
R¹⁰ is selected from the group consisting of H, C₁-C₈ alkyl optionally substituted with one or two —OR, and halo;
R¹¹ is selected from the group consisting of H, C₁-C₈ alkyl, —OR and halo;
R¹² is —C(O)N(R)₂ or —C(O)NHR;
R¹³ is H;
each R is independently selected from the group consisting of H or C₁-C₈ alkyl, or C₁-C₈ haloalkyl, or two R join to form, together with the atom or atoms to which they are bound, a —C₃-C₁₀ cycloalkyl or 3-10 membered heterocycle,
where said 3-10 membered heterocycle contains one, two or three atoms selected from N, O and S; and
where, when two R join to form, together with the atom or atoms to which they are bound, a —(C₃-C₁₀) cycloalkyl or 3-10 membered heterocycle, said —C₃-C₁₀ cycloalkyl or 3-10 membered heterocycle is optionally substituted with one or more substituents each independently selected from C₁-C₈ alkyl, hydroxy, C₁-C₈ alkoxy, —(C₃-C₁₀) cycloalkyl, 3-10 membered heterocycle, halo and cyano.

In one embodiment, the STING agonist is a compound selected from:

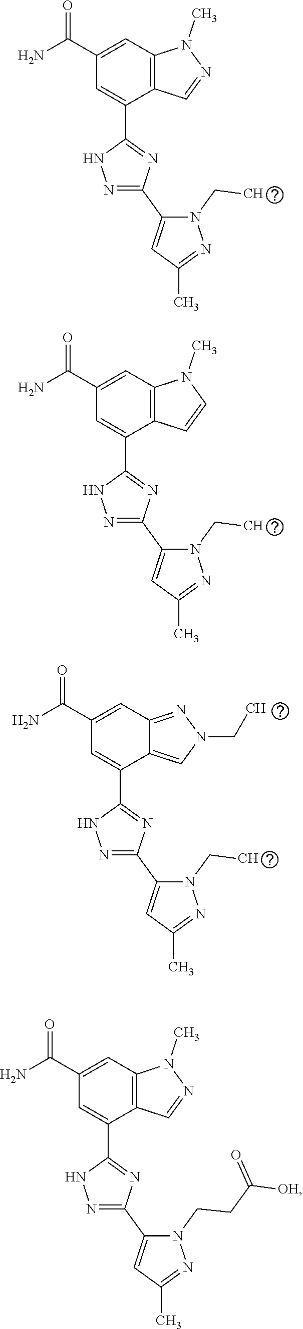 [SMILES: CCn1nc(C)cc1-c1n[nH]c(-c2cc(C(N)=O)cc3c2cnn3C)n1 ; CCn1nc(C)cc1-c1n[nH]c(-c2cc(C(N)=O)cc3c2ccn3C)n1 ; CCn1cc2c(-c3nc(-c4cc(C)nn4CC)n[nH]3)cc(C(N)=O)cc2n1 ; Cc1cc(-c2n[nH]c(-c3cc(C(N)=O)cc4c3cnn4C)n2)n(CCC(=O)O)n1]

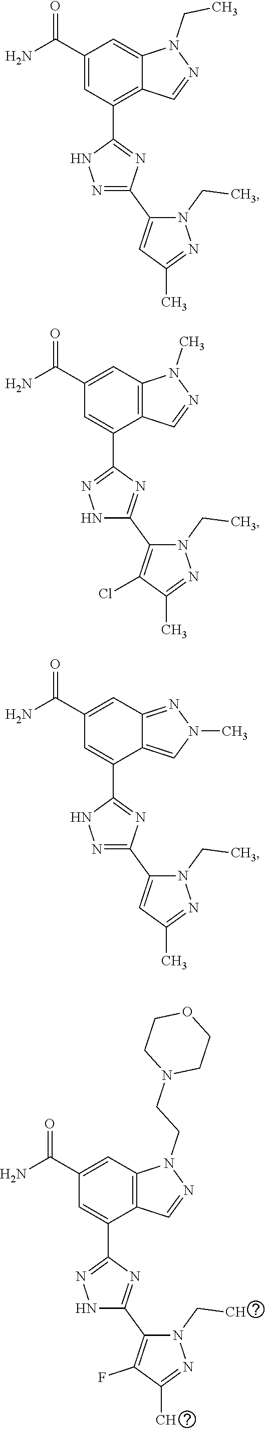

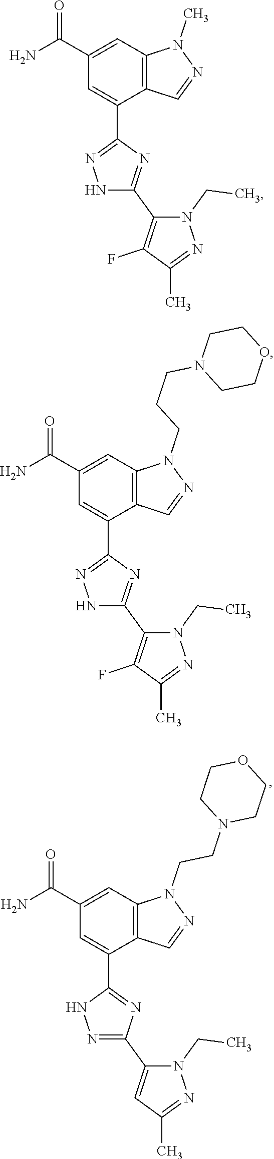 [SMILES: CCn1nc(C)c(F)c1-c1nc(-c2cc(C(N)=O)cc3c2cnn3C)n[nH]1 ; CCn1nc(C)c(F)c1-c1nc(-c2cc(C(N)=O)cc3c2cnn3CCCN2CCOCC2)n[nH]1 ; CCn1nc(C)cc1-c1n[nH]c(-c2cc(C(N)=O)cc3c2cnn3CCN2CCOCC2)n1]

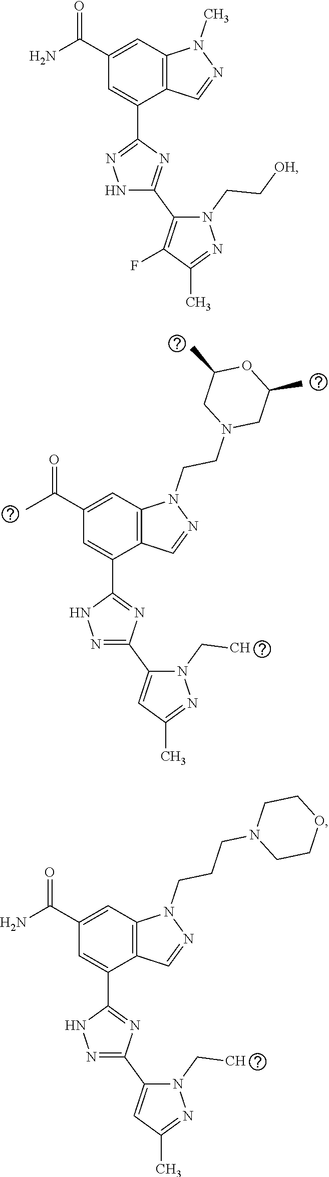

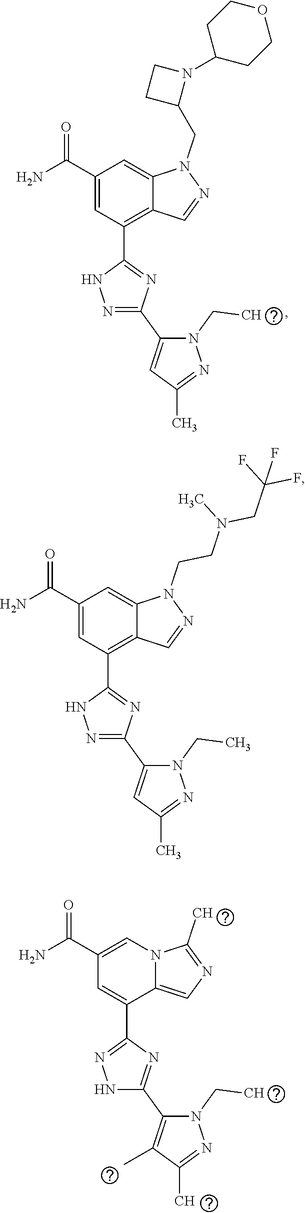 [SMILES: CCn1nc(C)cc1-c1n[nH]c(-c2cc(C(N)=O)cc3c2cnn3CC2CCN2C2CCOCC2)n1 ; CCn1nc(C)cc1-c1n[nH]c(-c2cc(C(N)=O)cc3c2cnn3CCN(C)CC(F)(F)F)n1 ; CCn1nc(C)c(C)c1-c1nc(-c2cc(C(N)=O)cn3c(C)ncc23)n[nH]1]

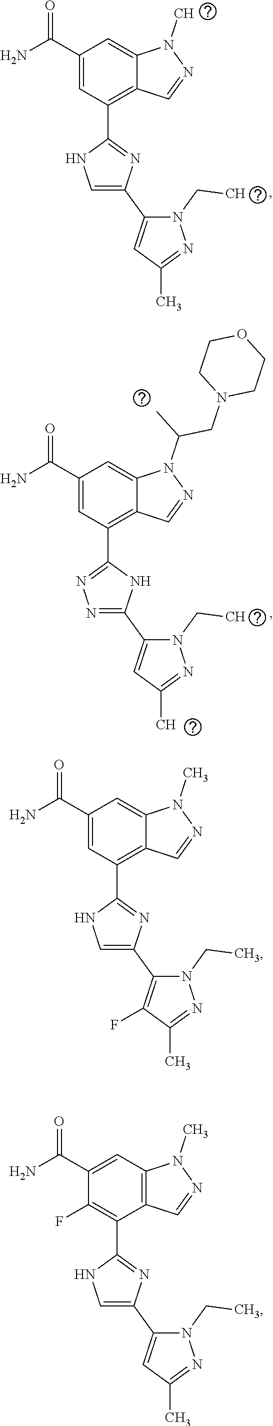 [SMILES: CCn1nc(C)cc1-c1c[nH]c(-c2cc(C(N)=O)cc3c2cnn3C)n1 ; CCn1nc(C)cc1-c1nnc(-c2cc(C(N)=O)cc3c2cnn3C(C)CN2CCOCC2)[nH]1 ; CCn1nc(C)c(F)c1-c1c[nH]c(-c2cc(C(N)=O)cc3c2cnn3C)n1 ; CCn1nc(C)cc1-c1c[nH]c(-c2c(F)c(C(N)=O)cc3c2cnn3C)n1]

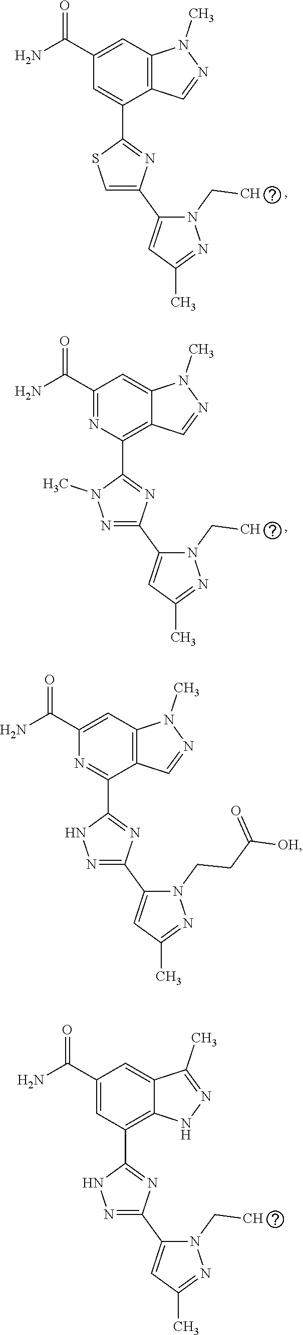 [SMILES: CCn1nc(C)cc1-c1csc(-c2cc(C(N)=O)cc3c2cnn3C)n1 ; CCn1nc(C)cc1-c1nc(-c2nc(C(N)=O)cc3c2cnn3C)n(C)n1 ; Cc1cc(-c2n[nH]c(-c3nc(C(N)=O)cc4c3cnn4C)n2)n(CCC(=O)O)n1 ; CCn1nc(C)cc1-c1n[nH]c(-c2cc(C(N)=O)cc3c(C)n[nH]c23)n1]

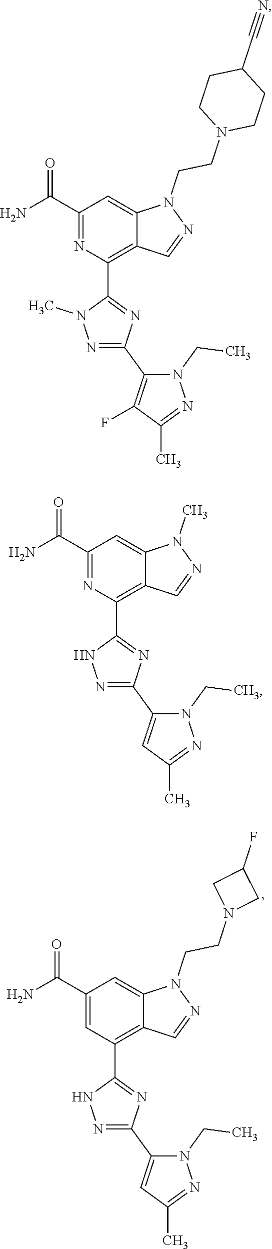 [SMILES: CCn1nc(C)c(F)c1-c1nc(-c2nc(C(N)=O)cc3c2cnn3CCN2CCC(C#N)CC2)n(C)n1 ; CCn1nc(C)cc1-c1n[nH]c(-c2nc(C(N)=O)cc3c2cnn3C)n1 ; CCn1nc(C)cc1-c1n[nH]c(-c2cc(C(N)=O)cc3c2cnn3CCN2CC(F)C2)n1]

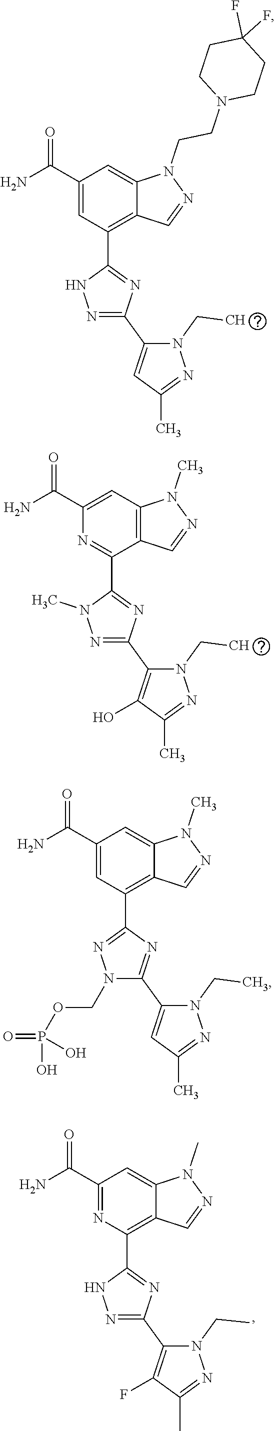

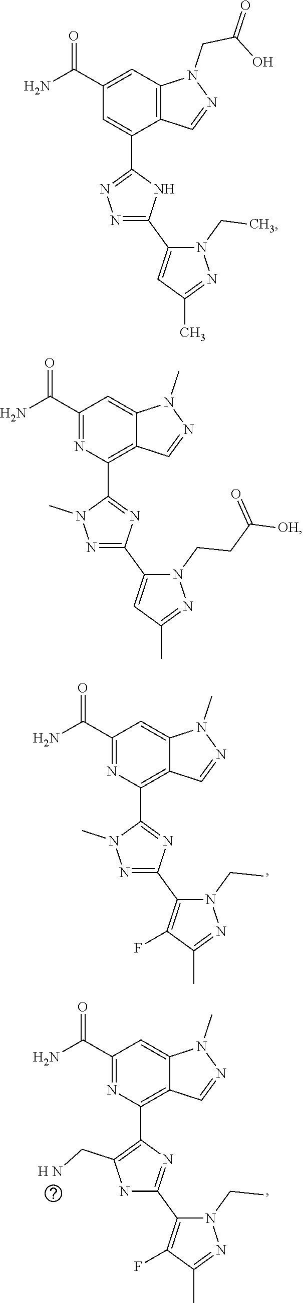

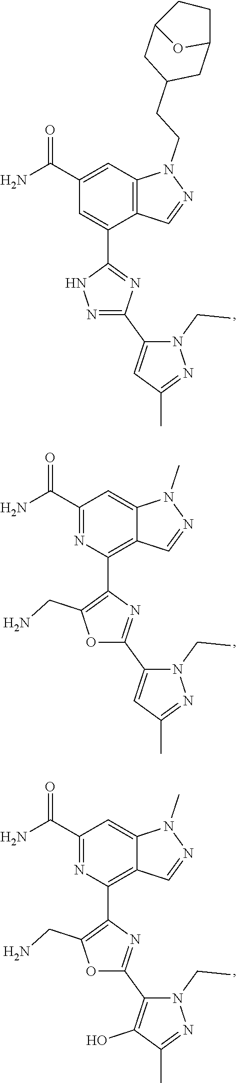

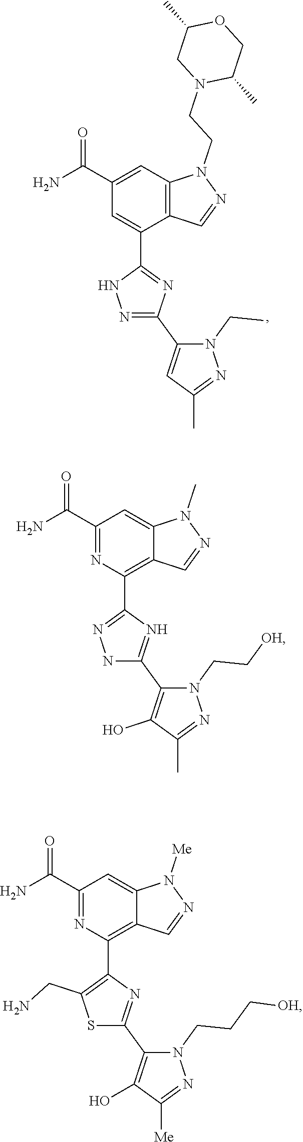 [SMILES: CCn1nc(C)cc1-c1n[nH]c(-c2cc(C(N)=O)cc3c2cnn3CCN2C[C@H](C)OC[C@@H]2C)n1 ; Cc1nn(CCO)c(-c2nc(-c3nc(C(N)=O)cc4c3cnn4C)n[nH]2)c1O ; Cc1nn(CCCO)c(-c2nc(-c3nc(C(N)=O)cc4c3cnn4C)c(CN)s2)c1O]

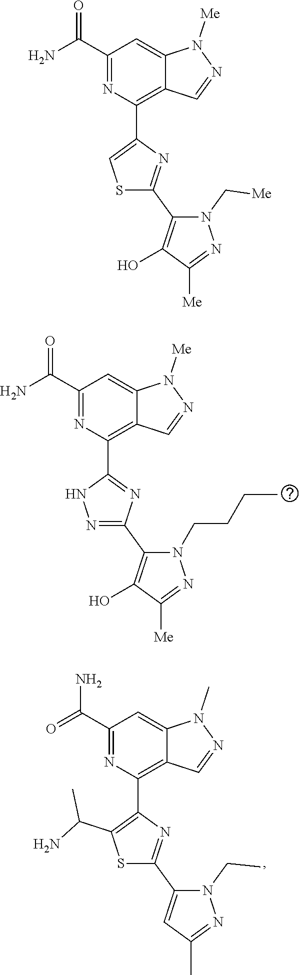 [SMILES: CCn1nc(C)c(O)c1-c1nc(-c2nc(C(N)=O)cc3c2cnn3C)cs1 ; CCCCn1nc(C)c(O)c1-c1n[nH]c(-c2nc(C(N)=O)cc3c2cnn3C)n1 ; CCn1nc(C)cc1-c1nc(-c2nc(C(N)=O)cc3c2cnn3C)c(C(C)N)s1]

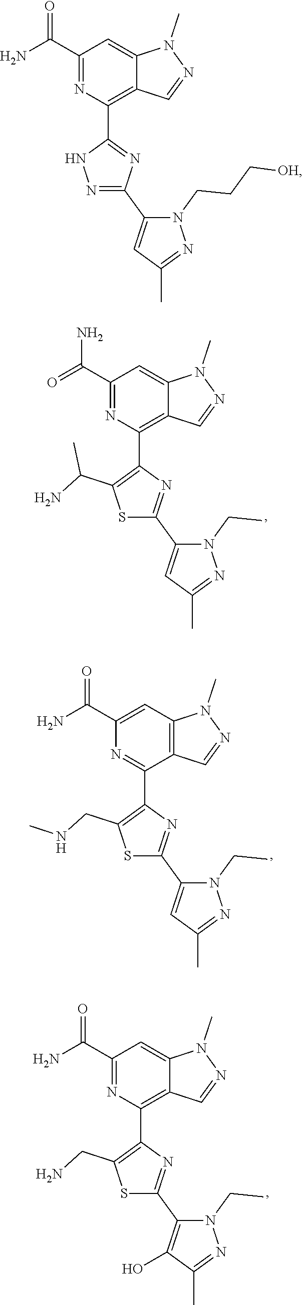

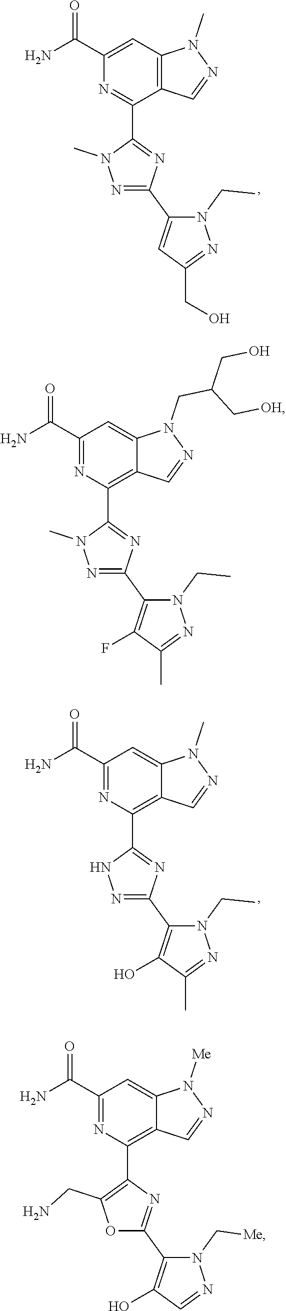

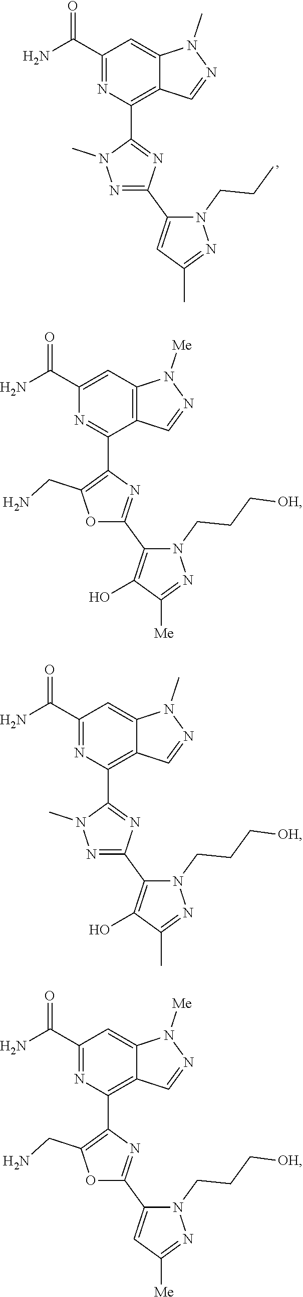

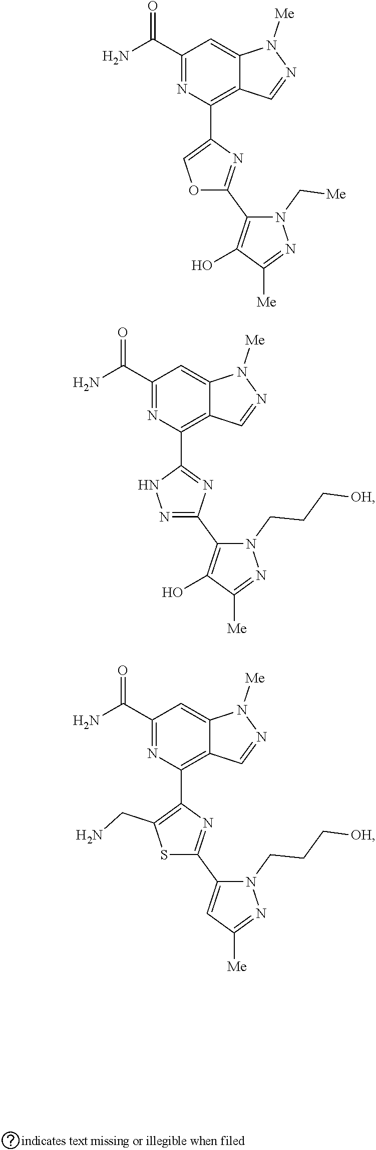

In one embodiment, the STING agonist is a compound of Formula C:

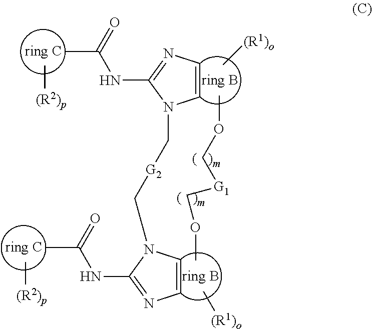

wherein
G₁ is independently selected from Ring A

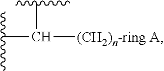

ring A is independently selected from optionally substituted heterocyclyl and optionally substituted heteroaryl,
ring B is aromatic carbocyclic ring,
ring C is optionally substituted five-membered heteroaryl,
R¹ is —CON(R³)₂,
R² is independently selected from hydrogen, optionally substituted C₁-C₆ alkyl, and optionally substituted C₃-C₅ monocyclic cycloalkyl,
R³ is independently selected from hydrogen, and optionally substituted C₁-C₆ alkyl;
m is selected from 0, or 1;
n is selected from 0, 1, or 2;
o is 1;
p is selected from 0, 1, or 2;
when ‘alkyl’s substituted, it is substituted with 1 to 4 substituents independently selected from halogen, alkyl, perhaloalkyl, cycloalkyl, heterocyclyl, —N(R⁴)₂, and —OR⁴;
when ‘carbocycle’ or ‘cycloalkyl’ is substituted, it is substituted with 1 to 4 substituents independently selected from halogen, alkyl, perhaloalkyl, —N(R⁴)₂, and —OR⁴;
when ‘heterocycle’ or ‘heterocyclyl’ is substituted, it is substituted with 1 to 4 substituents independently selected from oxo (═O), halogen, cyano, alkyl, perhaloalkyl, —OR⁴, —C(═O)OH, —OP(O)(OR⁴)₂, —P(O)(OR⁴)₂, —P(O)(OR⁴)R^{4a}, —SO₂R^{4a}, —SO₂NH₂, —C(═O)N(H)R⁴, —C(═O)N(alkyl)R⁴, —N(H)C(═O)R^{4a}, —N(H)R⁴, and —N(alkyl)R⁴;
when the ‘heteroaryl’ group is substituted, it is substituted with I to 4 substituents selected from halogen, cyano, alkyl, perhaloalkyl, —O-alkyl, —O-perhaloalkyl, —N(alkyl)alkyl, —N(H)R⁴, —SO₂-alkyl, —N(alkyl)C(═O)alkyl, —N(H)C(═O)alkyl, —C(═O)N(alkyl)alkyl, —C(═O)N(H)alkyl, —C(═O)NH₂, —SO₂N(alkyl)alkyl, —SO₂N(H)alkyl, —SO₂NH₂, —C(═O)OH, —OP(O)(OR⁴)₂, —P(O)(OR⁴)₂, and —P(O)(OR⁴)R^{4a};
each R⁴ is independently selected from hydrogen, alkyl, and cycloalkyl; and
each R^{4a} is independently selected from alkyl, and cycloalkyl.

In one embodiment, the STING agonist is selected from:

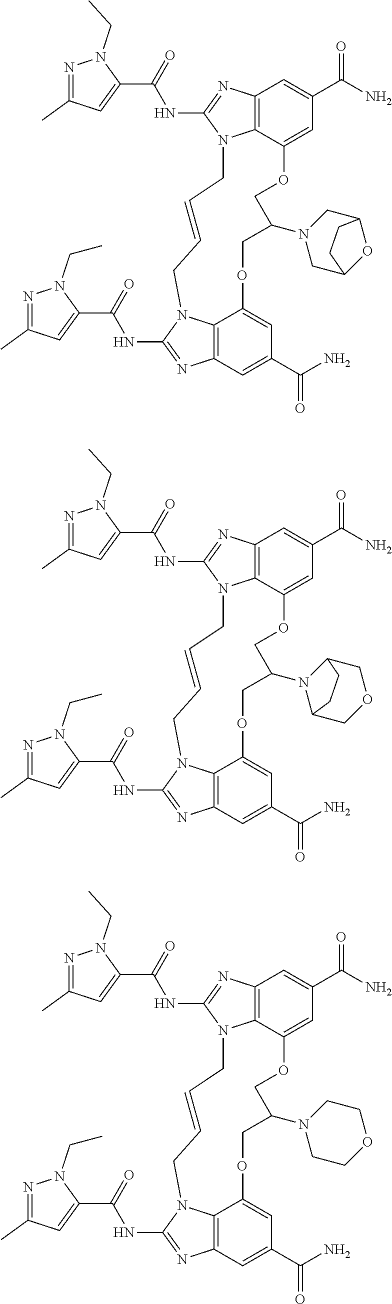 [SMILES: CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CC2CCC(C1)O2)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1C2CCC1COC2)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCOCC1)CO3]

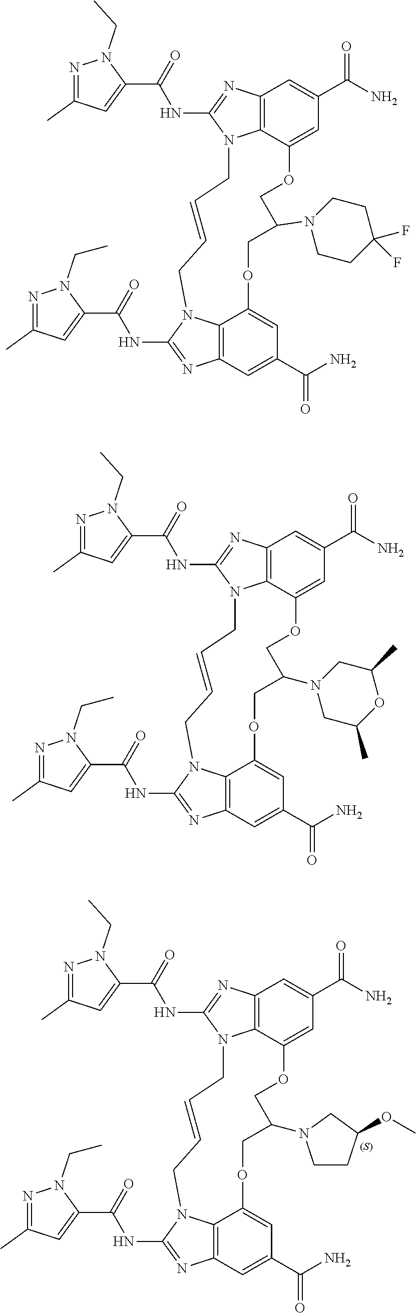

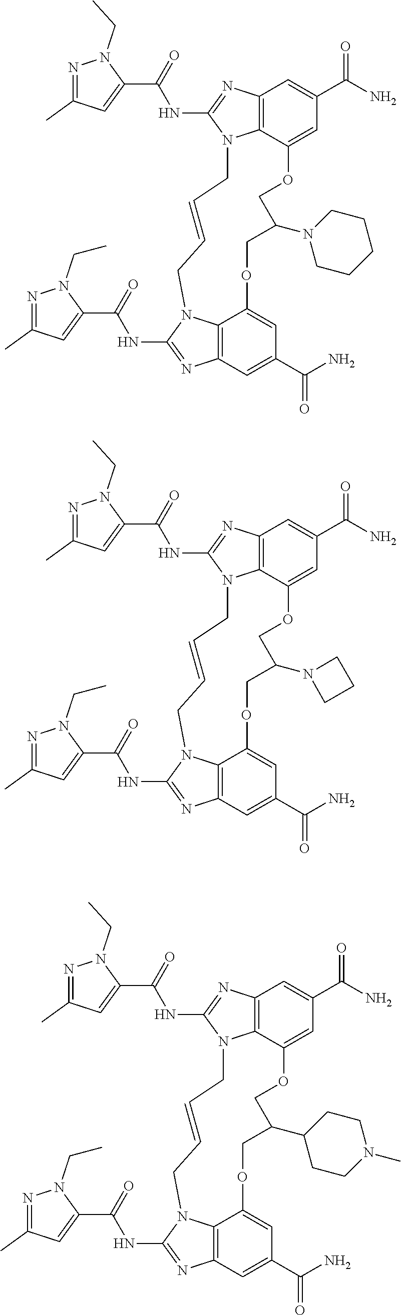

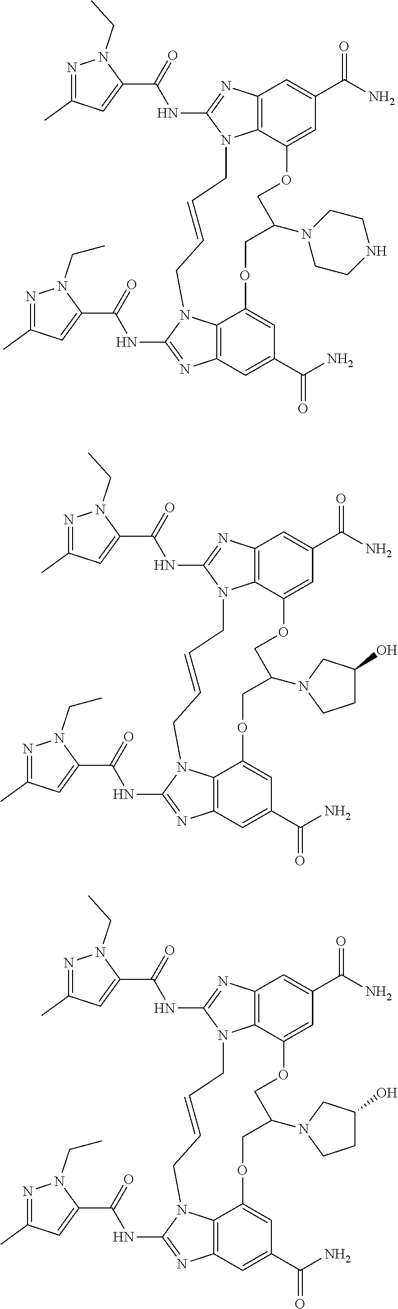 [SMILES: CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCNCC1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CC[C@H](O)C1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CC[C@@H](O)C1)CO3]

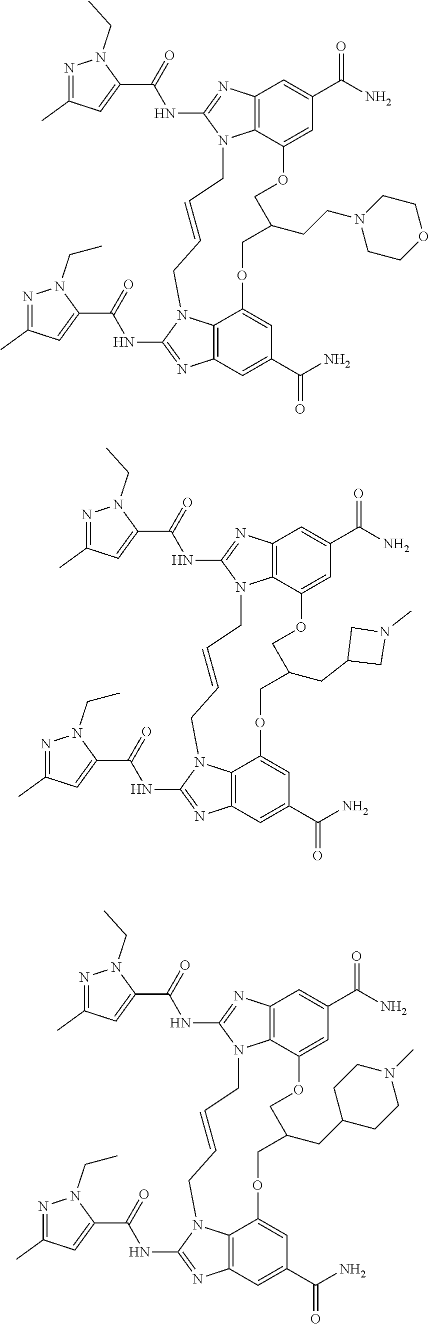

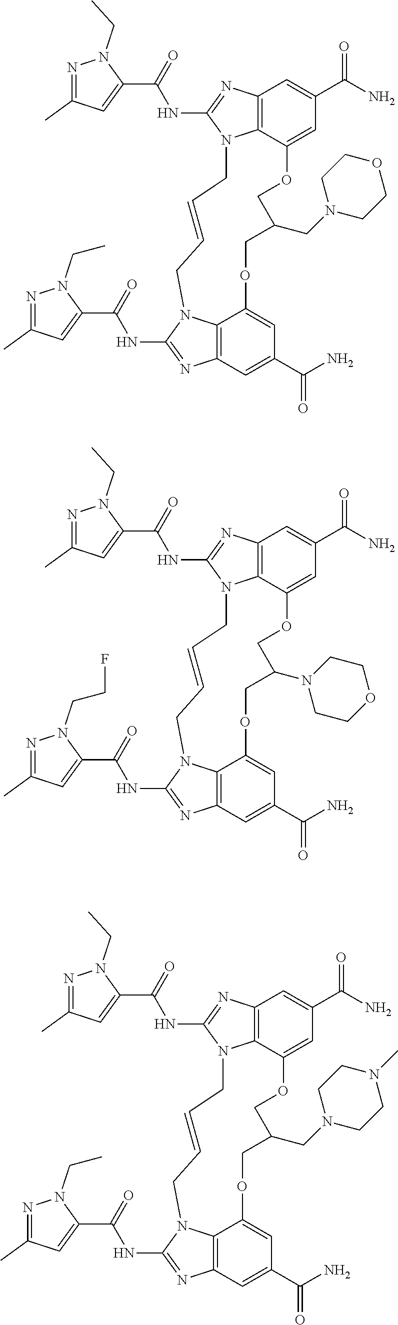

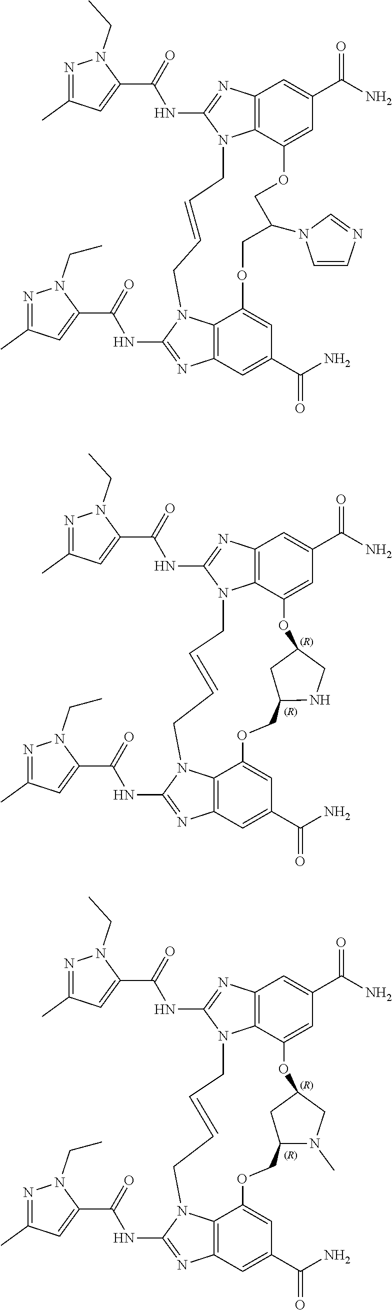

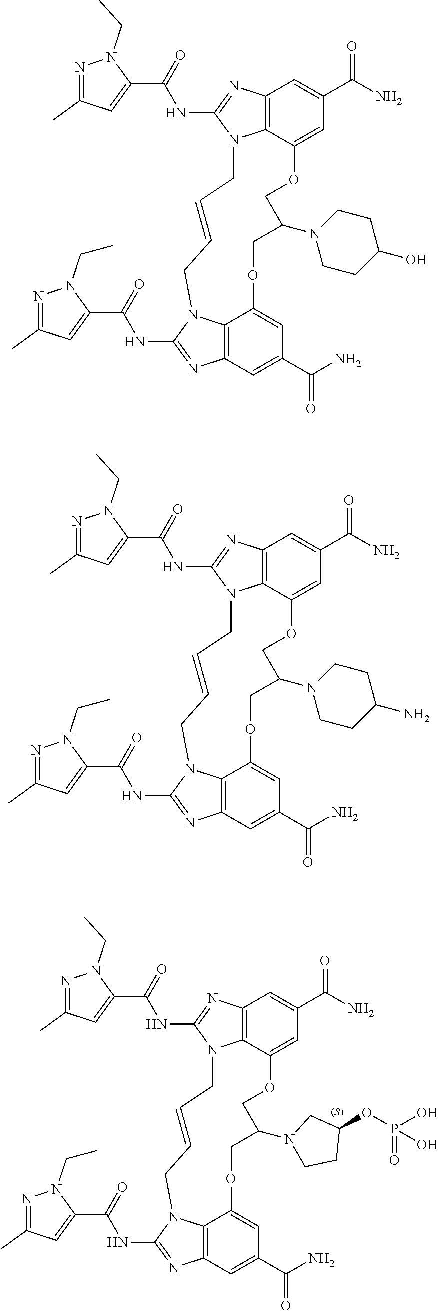

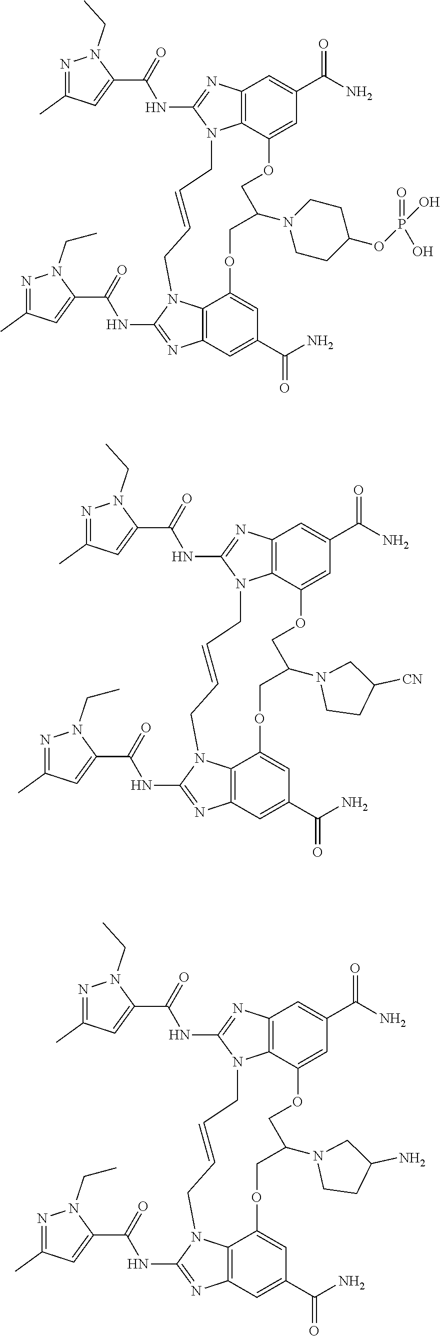 [SMILES: CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCC(OP(=O)(O)O)CC1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCC(C#N)C1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCC(N)C1)CO3]

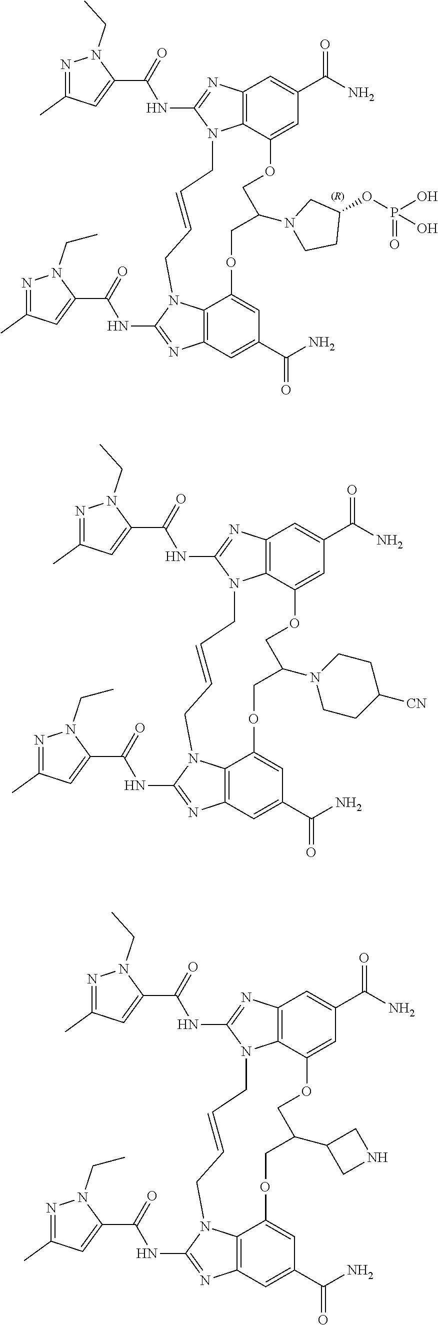

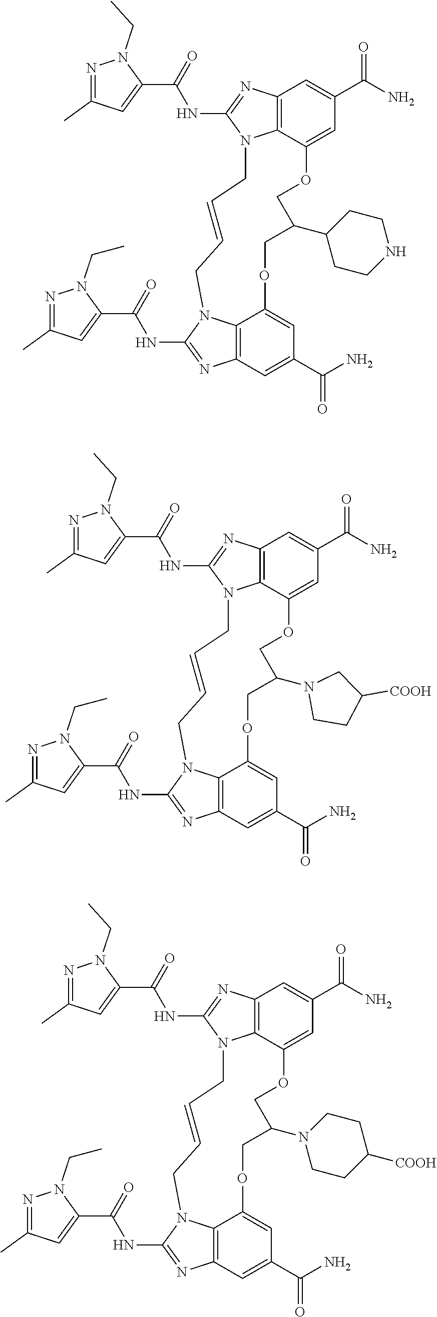 [SMILES: CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(C1CCNCC1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCC(C(=O)O)C1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCC(C(=O)O)CC1)CO3]

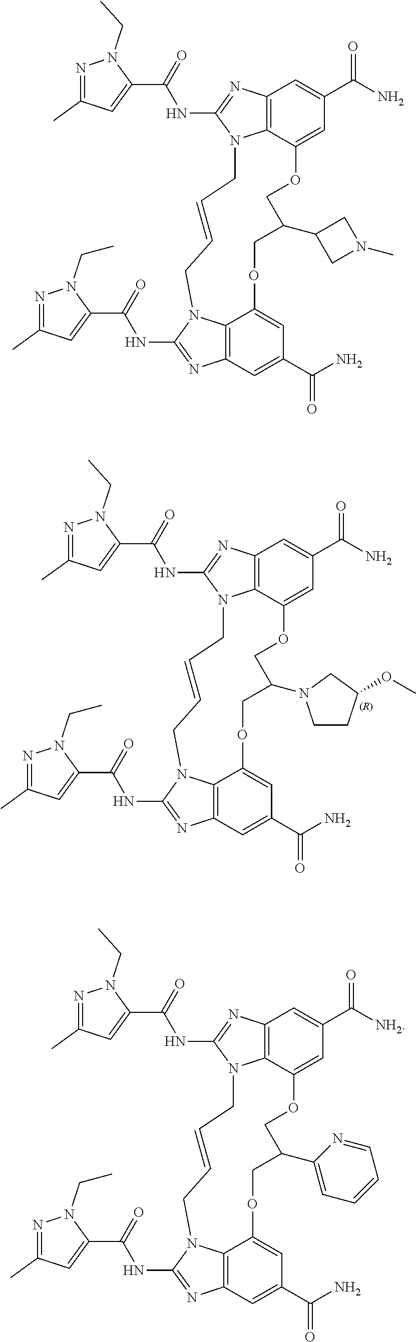

In one embodiment, the STING agonist is selected from IMSA101, ADU-S100 (MIW815), BMS-986301, CRD5500, CMA (IO-carboxymethyl-9-acridanone), diABZI STING agonist-1 (e.g., CAS No.: 2138299-34-8), DMXAA (ASA404/vadimezan), E7766 (Cas no. 2242635-02-3)

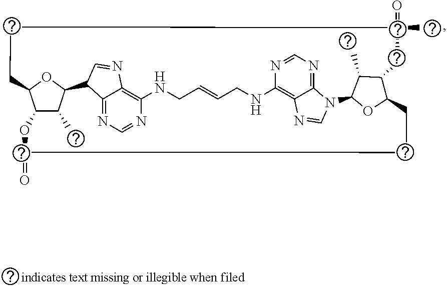

### MK-1454, MK-2118, SB-11285, SRCB-0074, TAK-676, and TTI-10001.

In one embodiment, the STING agonist is a STING agonist as disclosed in WO 2017/175147 (pages 7 to 92). In particular, the STING agonist is a compound according to Formula (I-N):

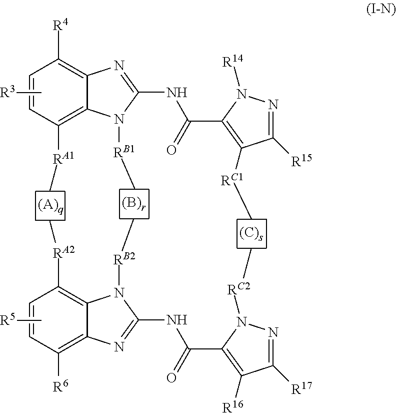

wherein:
q is 0 or 1;
r is 0 or 1;
s is 0 or 1;
wherein q+r+s=1 or 2;
when q is 0, R^{A1} and R^{A2} are each independently H, halogen, hydroxy, —O—P(O)(OH)₂, —O—P(O)(R^{I}R^{II})₂, —N(R^{e})(R^{f}), —CO₂R, —N(R^{f})COR^{b}, —N(R^{g})SO₂(C₁-C₄alkyl)-N(R^{e})(R^{f}), —N(R^{g})CO(C₁-C₄alkyl)-N(R^{h})(R^{f}), optionally substituted (C₁-C₆alkyl), optionally substituted (C₁-C₆alkyl)oxy-, optionally substituted (C₁-C₆alkyl)amino-, and optionally substituted (C₁-C₆alkyl)(C₁-C₄alkyl)amino-,
wherein the (C₁-C₆alkyl) of said optionally substituted (C₁-C₆alkyl), optionally substituted (C₁-C₆alkyl)oxy-, optionally substituted (C₁-C₆alkyl)amino- and optionally substituted (C₁-C₆alkyl)(C₁-C₄alkyl)amino- is optionally substituted by 1-4 substituents each independently selected from hydroxy, —O—P(O)(OH)₂, —O—P(O)(R^{I}R^{II})₂, C₁-C₄alkoxy-, —N(R^{e})(R^{f}), —CO₂(R^{f}), —CON(R^{e})(R^{f}), optionally substituted phenyl, optionally substituted 5-6 membered heterocycloalkyl and optionally substituted 5-6 membered heteroaryl group, wherein said optionally substituted phenyl, 5-6 membered heterocycloalkyl or 5-6 membered heteroaryl is optionally substituted by 1-4 substituents each independently selected from halogen, hydroxy, —O—P(O)(OH)₂, —O—P(O)(R^{I}R^{II})₂, amino, (C₁-C₆alkyl)amino-, (C₁-C₆alkyl)(C₁-C₆alkyl)amino-, —(C₁-C₆alkyl)-NH₂, halo(C₁-C₆alkyl), hydroxy-(C₁-C₄alkyl)-, —(C₁-C₄alkyl)-O—P(O)(OH)₂, -(C₁-C₄alkyl)-O—P(O)(R^{I}R^{II})₂, halo(C₁-C₄alkoxy)-, C₁-C₄alkoxy-, hydroxy-(C₂-C₄alkoxy)-, —(C₂-C₄alkoxy)-O—P(O)(OH)₂, —(C₂-C₄alkoxy)-O—P(O)(R^{I}R^{II})₂, —C₁-C₄alkyl-(C₁-C₄alkoxy) or C₁-C₄alkoxy-(C₁-C₄alkoxy)-;
when r is 0, R^{B1} and R^{B2} are each independently H, optionally substituted C₁-C₆alkyl, halo(C₁-C₆alkyl), optionally substituted C₂-C₆alkenyl, optionally substituted C₂-C₆alkynyl, optionally substituted C₃-C₆cycloalkyl, optionally substituted 4-6 membered heterocycloalkyl, optionally substituted phenyl, optionally substituted 5-6 membered heteroaryl, or optionally substituted 9-10 membered heteroaryl, wherein said optionally substituted C₁-C₆alkyl, optionally substituted C₂-C₆alkenyl, optionally substituted C₂-C₆alkynyl, optionally substituted C₃-C₆cycloalkyl, optionally substituted 4-6 membered heterocycloalkyl, optionally substituted phenyl, optionally substituted 5-6 membered heteroaryl, or optionally substituted 9-10 membered heteroaryl is optionally substituted by 1-4 substituents each independently selected from halogen, nitro, —R^{c}, —OH, —O—P(O)(OH)₂, —O—P(O)(R^{I}R^{II})₂, —OR^{c}, —NH₂, —NR^{c}R^{c}, —NR^{c}R^{d}, —OCOR^{c}, —CO₂H, —CO₂R^{c}, —SOR^{c}, —SO₂R^{c}, —CONH₂, —CONR^{c}R^{d}, —SO₂NH₂, —SO₂NR^{c}R^{d}, —OCONH₂, —OCONR^{c}R^{d}, —NR^{d}COR^{c}, —NR^{d}SOR^{c}, —NR^{d}CO₂R^{c}, and —NR^{d}SO₂R^{c};
when s is 0, R^{c1} is H, halogen, or C₁-C₄alkyl and R^{c2} is optionally substituted C₁-C₄alkyl, wherein said optionally substituted C₁-C₄alkyl group is optionally substituted by a substituent selected from —OR^{c}, —NR^{c}R^{d}, —CO₂R^{c}, —CONR^{c}R^{d}, —SO₂NR^{c}R^{d}, and —OCONR^{c}R^{d};
when q is 1, R^{A1} and R^{A2} are each independently —CH₂—, —NR^{e}—, or —O—, and A, taken together with R^{A1} and R^{A2}, forms a linking group, wherein A is -halo(C₁-C₁₂alkyl)-, optionally substituted —C₁-C₁₂alkyl-, optionally substituted —C₂-C₁₂alkenyl-, optionally substituted —C₂-C₁₂alkynyl-, optionally substituted —C₁-C₆alkyl-O—C₁-C₆alkyl-, optionally substituted —C₁-C₆alkyl-NR^{a}—C₁-C₆alkyl-, optionally substituted —C₁-C₆alkyl-(C₃-C₆cycloalkyl)-C₁-C₆alkyl-, optionally substituted —C₁-C₆alkyl-phenyl-C₁-C₆alkyl-, optionally substituted —C₁-C₆alkyl-(4-6 membered heterocycloalkyl)-C₁-C₆alkyl-, or optionally substituted —C₁-C₆alkyl-(5-6 membered heteroaryl)-C₁-C₆alkyl-, wherein the alkyl moiety of said optionally substituted —C₁-C₁₂alkyl-, optionally substituted —C₂-C₁₂alkenyl-, optionally substituted —C₂-C₁₂alkynyl-, optionally substituted —C₁-C₆alkyl-O—C₁-C₆alkyl-, optionally substituted —C₁-C₆alkyl-NR^{a}—C₁-C₆alkyl-, optionally substituted —C₁-C₆alkyl-(C₃-C₆cycloalkyl)-C₁-C₆alkyl-, optionally substituted —C₁-C₆alkyl-phenyl-C₁-C₆alkyl-, optionally substituted —C₁-C₆alkyl-(4-6 membered heterocycloalkyl)-C₁-C₆alkyl-, or optionally substituted —C₁-C₆alkyl-(5-6 membered heteroaryl)-C₁-C₆alkyl- is optionally substituted by 1-4 substituents each independently selected from halogen, halo(C₁-C₄alkyl), —OH, —O—P(O)(OH)₂, —O—P(O)(R^{I}R^{II})₂, —OR^{c}, —NH₂, —NR^{c}R^{d}, —OCOR^{c}, —CO₂H, —CO₂R^{c}, —SOR^{c}, —SO₂R^{c}, —CONH₂, —CONR^{c}R^{d}, —SO₂NH₂, —SO₂NR^{c}R^{d}, —OCONH₂, —OCONR^{c}R^{d}, —NR^{d}COR^{c}, —NR^{d}SOR^{c}, —NR^{d}CO₂R^{c}, and —NR^{d}SO₂R^{c}, and the C₃-C₆cycloalkyl, phenyl, 4-6 membered heterocycloalkyl, or 5-6 membered heteroaryl moiety of said optionally substituted —C₁-C₆alkyl-(C₃-C₆cycloalkyl)-C₁-C₆alkyl-, optionally substituted —C₁-C₆alkyl-phenyl-C₁-C₆alkyl-, optionally substituted —C₁-C₆alkyl-(4-6 membered heterocycloalkyl)-C₁-C₆alkyl-, or optionally substituted —C₁-C₆alkyl-(5-6 membered heteroaryl)-C₁-C₆alkyl- is optionally substituted by 1-4 substituents each independently selected from halogen, hydroxy, —O—P(O)(OH)₂, —O—P(O)(R^{I}R^{II})₂, amino, (C₁-C₄alkyl)amino-, (C₁-C₄alkyl)(C₁-C₄alkyl)amino-, C₁-C₄alkyl, halo(C₁-C₄alkyl), halo(C₁-C₄alkoxy)-, C₁-C₄alkoxy-, hydroxy-(C₁-C₄alkoxy)-, —(C₁-C₄alkoxyl)-O—P(O)(OH)₂, —(C₁-C₄alkoxyl)-O—P(O)(R^{I}R^{II})₂ and C₁-C₄alkoxy-(C₁-C₄alkoxy)-;
when r is 1, R^{B1} and R^{B2} are each independently —CH₂—, and B, taken together with R^{B1} and R^{B2}, forms a linking group, wherein B is a bond or B is -halo(C₁-C₁₀alkyl)-, optionally substituted —C₁-C₁₀alkyl-, optionally substituted —C₂-C₁₀alkenyl-, optionally substituted —C₂-C₁₀alkynyl-, optionally substituted —C₁-C₆alkyl-O—C₁-C₆alkyl-, optionally substituted —C₁-C₆alkyl-NR^{a}—C₁-C₆alkyl-, optionally substituted C₃-C₆cycloalkyl, optionally substituted phenyl, optionally substituted 4-6 membered heterocycloalkyl, optionally substituted 5-6 membered heteroaryl, optionally substituted —C₁-C₄alkyl-(C₃-C₆cycloalkyl)-C₁-C₄alkyl-, optionally substituted —C₁-C₄alkyl-phenyl-C₁-C₄alkyl-, optionally substituted —C₁-C₄alkyl-(4-6 membered heterocycloalkyl)-C₁-C₄alkyl-, or optionally substituted —C₁-C₄alkyl-(5-6 membered heteroaryl)-C₁-C₄alkyl-, wherein the alkyl moiety of said optionally substituted —C₁-C₁₀alkyl-, optionally substituted —C₂-C₁₀alkenyl-, optionally substituted —C₂-C₁₀alkynyl-, optionally substituted —C₁-C₆alkyl-O—C₁-C₆alkyl-, optionally substituted —C₁-C₆alkyl-NR^{a}—C₁-C₆alkyl-, optionally substituted —C₁-C₄alkyl-(C₃-C₆cycloalkyl)-C₁-C₄alkyl-, optionally substituted —C₁-C₄alkyl-phenyl-C₁-C₄alkyl-, optionally substituted —C₁-C₄alkyl-(4-6 membered heterocycloalkyl)-C₁-C₄alkyl-, or optionally substituted —C₁-C₄alkyl-(5-6 membered heteroaryl-C₁-C₄alkyl)- is optionally substituted by 1 or 2 substituents each independently selected from halogen, halo(C₁-C₄alkyl), —OH, —O—P(O)(OH)₂, —O—P(O)(R_{I}R_{II})₂, —OR^{c}, —NH₂, —NR^{c}R^{d}, —OCOR^{c}, —CO₂H, —CO₂R^{c}, —SOR^{c}, —SO₂R^{c}, —CONH₂, —CONR^{c}R^{d}, —SO₂NH₂, —SO₂NR^{c}R^{d}, —OCONH₂, —OCONR^{c}R^{d}, —NR^{d}COR^{c}, —NR^{d}SOR^{c}, —NR^{d}CO₂R^{c}, and —NR^{d}SO₂R^{c}, and the C₃-C₆cycloalkyl, phenyl, 4-6 membered heterocycloalkyl, or 5-6 membered heteroaryl moiety of said optionally substituted C₃-C₆cycloalkyl, optionally substituted phenyl, optionally substituted 4-6 membered heterocycloalkyl, optionally substituted 5-6 membered heteroaryl, optionally substituted —C₁-C₄alkyl-(C₃-C₆cycloalkyl)-C₁-C₄alkyl-, optionally substituted —C₁-C₄alkyl-phenyl-C₁-C₄alkyl-, optionally substituted —C₁-C₄alkyl-(4-6 membered heterocycloalkyl)-C₁-C₄alkyl-, or optionally substituted —C₁-C₄alkyl-(5-6 membered heteroaryl)-C₁-C₄alkyl- is optionally substituted by 1-4 substituents each independently selected from halogen, hydroxy, —O—P(O)(OH)₂, —O—P(O)(R^{I}R^{II})₂, amino, (C₁-C₄alkyl)amino-, (C₁-C₄alkyl)(C₁-C₄alkyl)amino-, C₁-C₄alkyl, halo(C₁-C₄alkyl), halo(C₁-C₄alkoxy)-, C₁-C₄alkoxy-, hydroxy-(C₂-C₄alkoxy)-, —(C₂-C₄alkoxy)O—P(O)(OH)₂, —(C₂-C₄alkoxy)-O—P(O)(R^{I}R^{II})₂, and C₁-C₄alkoxy-(C₁-C₄alkoxy)-;
when s is 1, R^{c}1 and R^{C2} are each independently —CH₂—, and C, taken together with R^{c}1 and R^{c2}, forms a linking group, wherein C is -halo(C₁-C₁₂alkyl)-, optionally substituted —C₁-C₁₂alkyl-, optionally substituted —C₂-C₁₂alkenyl-, optionally substituted —C₂-C₁₂alkynyl-, optionally substituted —C₁-C₆alkyl-O—C₁-C₆alkyl-, optionally substituted —C₁-C₆alkyl-NR^{a}—C₁-C₆alkyl-, optionally substituted —C₁-C₆alkyl-(C₃-C₆cycloalkyl)-C₁-C₆alkyl-, optionally substituted —C₁-C₆alkyl-phenyl-C₁-C₆alkyl-, optionally substituted —C₁-C₆alkyl-(4-6 membered heterocycloalkyl)-C₁-C₆alkyl-, or optionally substituted —C₁-C₆alkyl-(5-6 membered heteroaryl)-C₁-C₆alkyl-, wherein the alkyl moiety of said optionally substituted —C₁-C₁₂alkyl-, optionally substituted —C₂-C₁₂alkenyl-, optionally substituted —C₂-C₁₂alkynyl-, optionally substituted —C₁-C₆alkyl-O—C₁-C₆alkyl-, optionally substituted —C₁-C₆alkyl-NR^{a}—C₁-C₆alkyl-, optionally substituted —C₁-C₆alkyl-(C₃-C₆cycloalkyl)-C₁-C₆alkyl-, optionally substituted —C₁-C₆alkyl-phenyl-C₁-C₆alkyl-, optionally substituted —C₁-C₆alkyl-(4-6 membered heterocycloalkyl)-C₁-C₆alkyl-, or optionally substituted —C₁-C₆alkyl-(5-6 membered heteroaryl)-C₁-C₆alkyl- is optionally substituted by 1 or 2 substituents each independently selected from halogen, halo(C₁-C₄alkyl), —OH, —O—P(O)(OH)₂, —O—P(O)(R^{I}R^{II})₂, —OR^{c}, —NH₂, —NR^{c}R^{d}, —OCOR^{c}, —CO₂H, —CO₂R^{c}, —SOR^{c}, —SO₂R^{c}, —CONH₂, —CONR^{c}R^{d}, —SO₂NH₂, —SO₂NR^{c}R^{d}, —OCONH₂, —OCONR^{c}R^{d}, —NR^{d}COR^{c}, —NR^{d}SOR^{c}, —NR^{d}CO₂R^{c}, and —NR^{d}SO₂R^{c}, and the C₃-C₆cycloalkyl, phenyl, 4-6 membered heterocycloalkyl, or 5-6 membered heteroaryl moiety of said optionally substituted —C₁-C₆alkyl-(C₃-C₆cycloalkyl)-C₁-C₆alkyl-, optionally substituted —C₁-C₆alkyl-phenyl-C₁-C₆alkyl-, optionally substituted —C₁-C₆alkyl-(4-6 membered heterocycloalkyl)-C₁-C₆alkyl-, or optionally substituted —C₁-C₆alkyl-(5-6 membered heteroaryl)-C₁-C₆alkyl- is optionally substituted by 1-4 substituents each independently selected from halogen, hydroxy, —O—P(O)(OH)₂, —O—P(O)(R^{I}R^{II})₂, amino, (C₁-C₄alkyl)amino-, (C₁-C₄alkyl)(C₁-C₄alkyl)amino-, C₁-C₄alkyl, halo(C₁-C₄alkyl), halo(C₁-C₄alkoxy)-, C₁-C₄alkoxy-, hydroxy-(C₂-C₄alkoxy)-, —(C₂-C₄alkoxy)-O—P(O)(OH)₂, —(C₂-C₄alkoxy)-O—P(O)(R^{I}R^{II})₂,and C₁-C₄alkoxy-(C₁-C₄alkoxy)-;
R³ and R⁵ are each independently —CON(R^{d})(R), or one of R³ and R⁵ is —CON(R^{d})(R), and the other of R³ and R⁵ is H, COOH or —CO₂(R^{c}); R⁴ and R⁶ are each independently selected from H, halogen, halo(C₁-C₆alkyl), halo(C₁-C₆alkoxy)-, hydroxy, —O—P(O)(OH)₂, —O—P(O)(R^{I}R^{II})₂, —NH₂, —NR^{c}R^{c}, —NR^{c}R^{d}, —COR^{c}, —CO₂R^{c}, —N(R^{d})COR^{c}, —N(R^{d})SO₂R^{c}, —N(R^{g})SO₂(C₁-C₂alkyl)-N(R^{h})(R^{f}), —N(R^{g})CO(C₁-C₂alkyl)-N(R^{h})(R^{f}), optionally substituted (C₁-C₆alkyl), optionally substituted (C₁-C₆alkyl)oxy-, optionally substituted (C₁-C₆alkyl)amino-, and optionally substituted (C₁-C₆alkyl)(C₁-C₄alkyl)amino-, wherein the (C₁-C₆alkyl) of said optionally substituted (C₁-C₆alkyl), optionally substituted (C₁-C₆alkyl)oxy-, optionally substituted (C₁-C₆alkyl)amino- and optionally substituted (C₁-C₆alkyl)(C₁-C₄alkyl)amino- is optionally substituted by 1-4 substituents each independently selected from —OH, —O—P(O)(OH)₂, —O—P(O)(R^{I}R^{II})₂, —OR^{c}, —NH₂, —NR^{c}R^{c}, —NR^{c}R^{d}, —CO₂H, —CO₂R^{c}, —OCOR^{c}, —CO₂H, —CO₂R^{c}, —SOR^{c}, —SO₂R^{c}, —CONH₂, —CONR^{c}R^{d}, —SO₂NH₂, —SO₂NR^{c}R^{d}, —OCONH₂, —OCONR^{c}R^{d}, —NR^{d}COR^{c}, —NR^{d}SOR^{c}, —NR^{d}CO₂R^{c}, —NR^{d}SO₂R^{c}, optionally substituted phenyl, optionally substituted 5-6 membered heterocycloalkyl and optionally substituted 5-6 membered heteroaryl group, wherein said optionally substituted phenyl, 5-6 membered heterocycloalkyl or 5-6 membered heteroaryl is optionally substituted by 1-4 substituents each independently selected from halogen, hydroxy, —O—P(O)(OH)₂, —O—P(O)(R^{I}R^{II})₂, amino, (C₁-C₄alkyl)amino-, (C₁-C₄alkyl)(C₁-C₄alkyl)amino-, C₁-C₄alkyl, halo(C₁-C₄alkyl), hydroxy-(C₁-C₄alkyl)-, —(C₁-C₄alkyl)-O—P(O)(OH)₂, —(C₁-C₄alkyl)-O—P(O)(R^{I}R^{II})₂, halo(C₁-C₄alkoxy)-, C₁-C₄alkoxy-, hydroxy-(C₂-C₄alkoxy)-, —(C₂-C₄alkoxy)-O—P(O)(OH)₂, —(C₂-C₄alkoxy)-O—P(O)(R^{I}R^{II})₂, C₁-C₄alkoxy-(C₁-C₄alkoxy)-, —COR^{d}, —CON(R^{d})(R^{f}), and —CO₂R^{d};
R¹⁴ is optionally substituted C₁-C₄alkyl, wherein said optionally substituted C₁-C₄alkyl is optionally substituted by a substituent selected from —OR^{c}, —NR^{c}R^{d}, —CO₂R^{c}, —CONR^{c}R^{d}, —SO₂NR^{c}R^{d}, and —OCONR^{c}R^{d};
R¹⁶ is H, halogen, or C₁-C₄alkyl;
R¹⁵ and R¹⁷ are each independently H, cyclopropyl, or C₁-C₄alkyl; Ra is H, —R^{c}, —COR^{c}, —CO₂H, —CO₂R^{c}, —SOR^{c}, —SO₂R^{c}, —CONH₂, —CONR^{c}R^{d}, —SO₂NH₂, or —SO₂NR^{c}R^{d};
each R^{b} is independently C₁-C₄alkyl, halo(C₁-C₄alkyl), —(C₁-C₄alkyl)-OH, —(C₁-C₄alkyl)-O—P(O)(OH)₂, —(C₁-C₄alkyl)-O—P(O)(R^{I}R^{II})₂, —(C₁-C₄alkyl)-O—(C₁-C₄alkyl), —(C₁-C₄alkyl)-N(R^{e})(R^{f}), —(C₁-C₄alkyl)-O—CO(C₁-C₄alkyl), or —(C₁-C₄alkyl)-CO—O—(C₁-C₄alkyl);
each R^{c} is independently C₁-C₄alkyl, halo(C₁-C₄alkyl), —(C₁-C₄alkyl)-OH, —(C₁-C₄alkyl)-O—P(O)(OH)₂, —(C₁-C₄alkyl)-O—P(O)(R^{I}R^{II})₂, —(C₁-C₄alkyl)-O—(C₁-C₄alkyl), —(C₁-C₄alkyl)-N(R^{e})(R^{f}), —(C₁-C₄alkyl)-O—CO(C₁-C₄alkyl), —(C₁-C₄alkyl)-CO—O—(C₁-C₄alkyl), optionally substituted C₃-C₆cycloalkyl, optionally substituted phenyl, optionally substituted 4-6 membered heterocycloalkyl, optionally substituted 5-6 membered heteroaryl, optionally substituted 9-10 membered heteroaryl, optionally substituted —C₁-C₄alkyl-C₃-C₆cycloalkyl, optionally substituted —C₁-C₄alkyl-phenyl, optionally substituted —C₁-C₄alkyl-4-6 membered heterocycloalkyl, optionally substituted —C₁-C₄alkyl-5-6 membered heteroaryl, or optionally substituted —C₁-C₄alkyl-9-10 membered heteroaryl, wherein the C₃-C₆cycloalkyl, phenyl, 4-6 membered heterocycloalkyl, 5-6 membered heteroaryl or 9-10 membered heteroaryl moiety of said optionally substituted C₃-C₆cycloalkyl, optionally substituted phenyl, optionally substituted 4-6 membered heterocycloalkyl, optionally substituted 5-6 membered heteroaryl, optionally substituted 9-10 membered heteroaryl optionally substituted —C₁-C₄alkyl-C₃-C₆cycloalkyl, optionally substituted —C₁-C₄alkyl-phenyl, optionally substituted —C₁-C₄alkyl-4-6 membered heterocycloalkyl, optionally substituted —C₁-C₄alkyl-5-6 membered heteroaryl, or optionally substituted —C₁-C₄alkyl-9-10 membered heteroaryl is optionally substituted by 1-4 substituents each independently selected from halogen, hydroxy, —O—P(O)(OH)₂, —O—P(O)(R^{I}R^{II})₂, amino, —(C₁-C₄alkyl)NH₂, (C₁-C₄alkyl)amino-, (C₁-C₄alkyl)(C₁-C₄alkyl)amino-, C₁-C₄alkyl, halo(C₁-C₄alkyl), halo(C₁-C₄alkoxy)-, C₁-C₄alkoxy-, hydroxy-(C₂-C₄alkoxy)-, —(C₂-C₄alkoxy)-O—P(O)(OH)₂, —(C₂-C₄alkoxy)-O—P(O)(R^{I}R^{II})₂, C₁-C₄alkoxy-(C₁-C₄alkoxy)-, —COR^{d}, —CON(R^{d})(R^{f}), and —CO₂R^{d};
each R^{d} is independently H or C₁-C₄alkyl;
each R^{e} is independently H, (C₁-C₄alkyl), —CO(C₁-C₄alkyl), —OCO(C₁-C₄alkyl), —CO₂(C₁-C₄alkyl), —(C₁-C₄alkyl)NH₂, —(C₁-C₄alkyl) C₁-C₄alkoxy, —CO-(optionally substituted 5-6 membered heterocycloalkyl), —CO(C₁-C₄alkyl)-(optionally substituted 5-6 membered heterocycloalkyl), —CO(optionally substituted 5-6 membered heteroaryl), —CO(C₁-C₄alkyl)-(optionally substituted 5-6 membered heteroaryl), wherein the optionally substituted 5-6 membered heterocycloalkyl or optionally substituted 5-6 membered heteroaryl is optionally substituted 1-4 substituents each independently selected from halogen, hydroxy, —O—P(O)(OH)₂, —O—P(O)(R^{I}R^{II})₂, amino, (C₁-C₄alkyl)amino-, (C₁-C₄alkyl)(C₁-C₄alkyl)amino-, C₁-C₄alkyl, halo(C₁-C₄alkyl), halo(C₁-C₄alkoxy)-, C₁-C₄alkoxy-, hydroxy-(C₂-C₄alkoxy)-, —(C₂-C₄alkoxy)O—P(O)(OH)₂, —(C₂-C₄alkoxy)-O—P(O)(R^{I}R^{II})₂, C₁-C₄alkoxy-(C₁-C₄alkoxy)-, —COR^{d}, —CON(R^{d})(R^{f}), and —CO₂R^{d};
each R^{f} is independently H or (C₁-C₄alkyl);
R^{g} and R^{h} are each independently H or (C₁-C₄alkyl) or R^{g} and R^{h}, taken together with the atom or atoms through which they are connected, form a 5-6 membered ring;
and each occurrence of R^{I} and R^{II} are independently (C₁-C₆alkyl)oxy-;
or a tautomer thereof.

In one embodiment, the STING compound is selected from:
(E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-hydroxypropoxy)-1H-benzo[d]imidazole-5-carboxamide (Example 10 of WO 2017/175147)

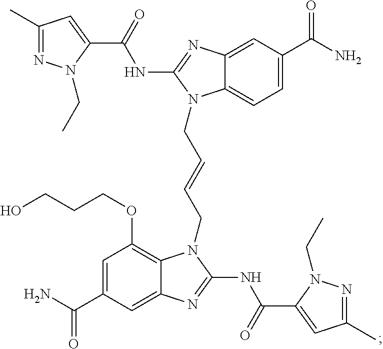

(E)-1-((E)-4-((E)-5-carbamoyl-2-((1-ethyl-3-methyl-1H-pyrazole-5-carbonyl)imino)-2,3-dihydro-1H-benzo[d]imidazol-1-yl)but-2-en-1-yl)-2-((1-ethyl-3-methyl-1H-pyrazole-5-carbonyl)imino)-7-(3-hydroxypropoxy)-2,3-dihydro-1H-benzo[d]imidazole-5-carboxamide (Example 10 of WO 2017/175147))

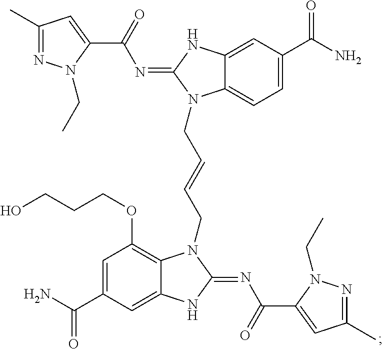

(Z)-1-((E)-4-((Z)-5-carbamoyl-2-((1-ethyl-3-methyl-1H-pyrazole-5-carbonyl)imino)-2,3-dihydro-1H-benzo[d]imidazol-1-yl)but-2-en-1-yl)-2-((1-ethyl-3-methyl-1H-pyrazole-5-carbonyl)imino)-7-(3-hydroxypropoxy)-2,3-dihydro-1H-benzo[d]imidazole-5-carboxamide (Example 10 of WO 2017/175147)

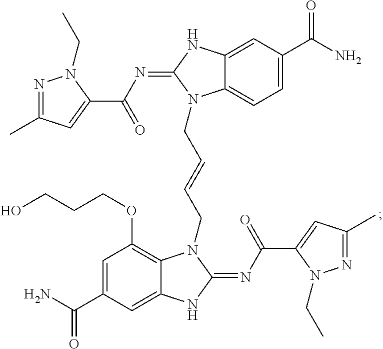

(E)-1-(4-(5-carbamoyl-2-(1-ethyl-3-methyl-1H-pyrazole-5-carboxamido)-7-(3-hydroxypropoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide (Example 11 of WO 2017/175147)

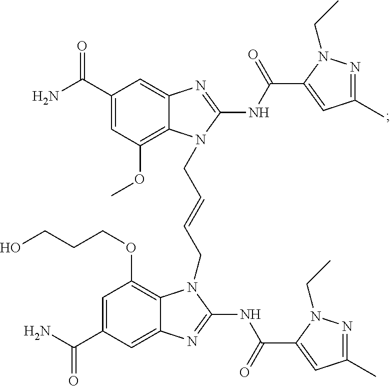 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCO)c21]

(E)-1-((E)-4-((E)-5-carbamoyl-2-((1-ethyl-3-methyl-1H-pyrazole-5-carbonyl)imino)-7-(3-hydroxypropoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide (Example 11 of WO 2017/175147)

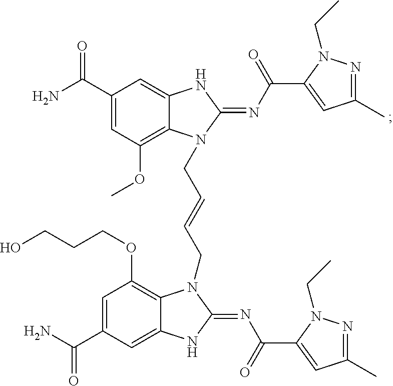 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCO)c21]

(Z)-1-((E)-4-((Z)-5-carbamoyl-2-((1-ethyl-3-methyl-1H-pyrazole-5-carbonyl)imino)-7-(3-hydroxypropoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide (Example 11 of WO 2017/175147)

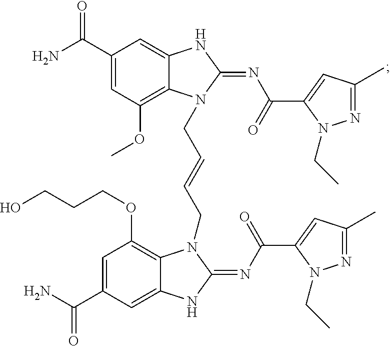 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCO)c21]

(E)-1-(4-(5-carbamoyl-2-(1-ethyl-3-methyl-1H-pyrazole-5-carboxamido)-17H-benzo[d]imidazol-1-yl)but-2-en-1-yl)-2-(1-ethyl-3-methyl-1H-pyrazole-5-carboxamido)-7-(3-morpholinopropoxy)-1H-benzo[d]imidazole-5-carboxamide (Example 13 of WO 2017/175147)

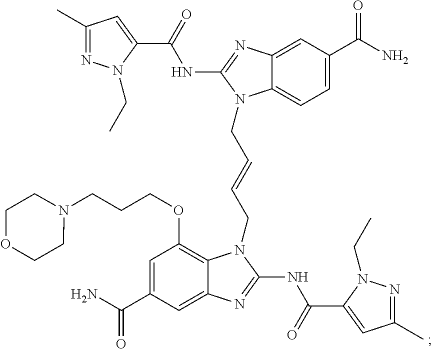 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCOCC3)c21]

(E)-1-((E)-4-((E)-5-carbamoyl-2-((1-ethyl-3-methyl-1H-pyrazole-5-carbonyl)imino)-2,3-dihydro-1H-benzo[d]imidazol-1-yl)but-2-en-1-yl)-2-((1-ethyl-3-methyl-1H-pyrazole-5-carbonyl)imino)-7-(3-morpholinopropoxy)-2,3-dihydro-1H-benzo[d]imidazole-5-carboxamide (Example 13 of WO 2017/175147)

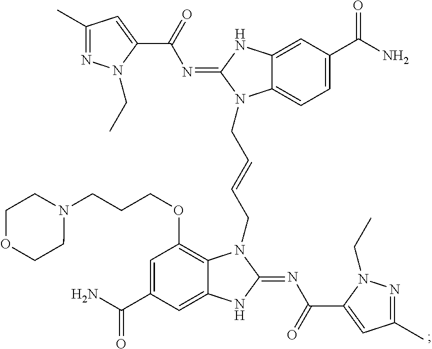 [SMILES: CCn1nc(C)cc1C(=O)/N=c1\[nH]c2cc(C(N)=O)ccc2n1C/C=C/Cn1/c(=N/C(=O)c2cc(C)nn2CC)[nH]c2cc(C(N)=O)cc(OCCCN3CCOCC3)c21]

(Z)-1-((E)-4-((Z)-5-carbamoyl-2-((1-ethyl-3-methyl-1H-pyrazole-5-carbonyl)imino)-2,3-dihydro-1H-benzo[d]imidazol-1-yl)but-2-en-1-yl)-2-((1-ethyl-3-methyl-1H-pyrazole-5-carbonyl)imino)-7-(3-morpholinopropoxy)-2,3-dihydro-1H-benzo[d]imidazole-5-carboxamide (Example 13 of WO 2017/175147)

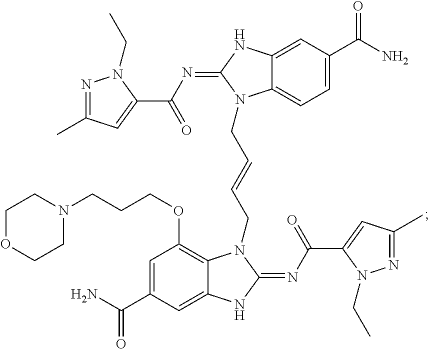 [SMILES: CCn1nc(C)cc1C(=O)/N=c1/[nH]c2cc(C(N)=O)ccc2n1C/C=C/Cn1/c(=N\C(=O)c2cc(C)nn2CC)[nH]c2cc(C(N)=O)cc(OCCCN3CCOCC3)c21]

(E)-1-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide (Example 14 of WO 2017/175147)

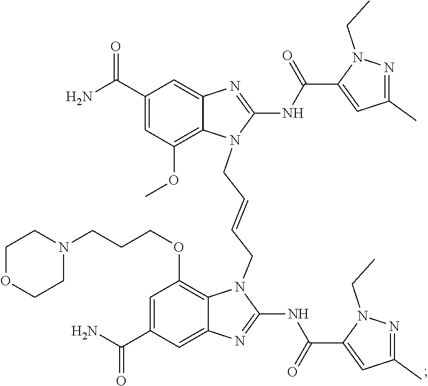 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCOCC3)c21]

(E)-1-((E)-4-((E)-5-carbamoyl-2-((1-ethyl-3-methyl-1H-pyrazole-5-carbonyl)imino)-7-(3-morpholinopropoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide (Example 14 of WO 2017/175147)

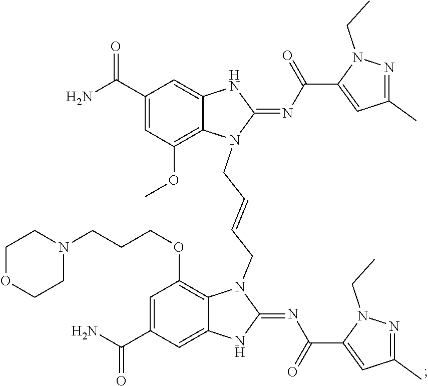 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCOCC3)c21]

(Z)-1-((E)-4-((Z)-5-carbamoyl-2-((1-ethyl-3-methyl-1H-pyrazole-5-carbonyl)imino)-7-(3-morpholinopropoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide (Example 14 of WO 2017/175147)

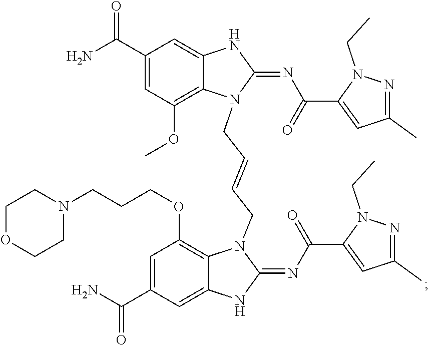 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCOCC3)c21]

3-(((Z)-6-carbamoyl-3-((E)-4-((Z)-5-carbamoyl-2-((1-ethyl-3-methyl-1H-pyrazole-5-carbonyl)imino)-7-methoxy-2,3-dihydro-1H-benzo[d]imidazol-1-yl)but-2-en-1-yl)-2-((1-ethyl-3-methyl-1H-pyrazole-5-carbonyl)imino)-2,3-dihydro-1H-benzo[d]imidazol-4-yl)oxy)propyldihydrogen phosphate (Example 19 of WO 2017/175147)

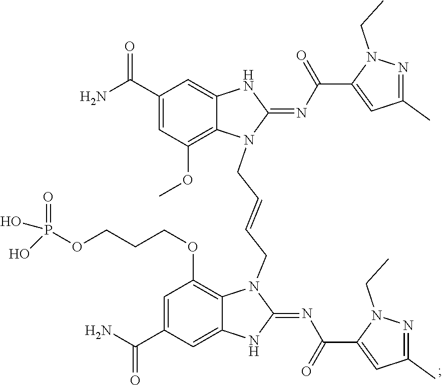 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCOP(=O)(O)O)c21]

(E)-3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl dihydrogen phosphate (Example 19 of WO 2017/175147)

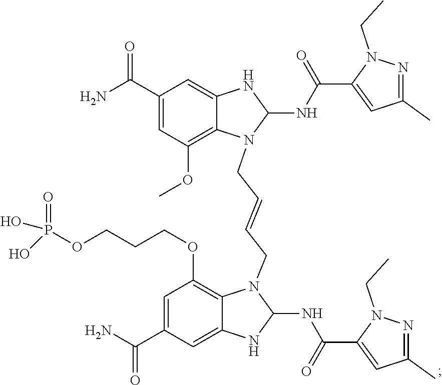 [SMILES: CCn1nc(C)cc1C(=O)NC1Nc2cc(C(N)=O)cc(OC)c2N1C/C=C/CN1c2c(cc(C(N)=O)cc2OCCCOP(=O)(O)O)NC1NC(=O)c1cc(C)nn1CC]

3-(((E)-6-carbamoyl-3-((E)-4-((E)-5-carbamoyl-2-((1-ethyl-3-methyl-1H-pyrazole-5-carbonyl)imino)-7-methoxy-2,3-dihydro-1H-benzo[d]imidazol-1-yl)but-2-en-1-yl)-2-((1-ethyl-3-methyl-1H-pyrazole-5-carbonyl)imino)-2,3-dihydro-1H-benzo[d]imidazol-4-yl)oxy) propyl dihydrogen phosphate (Example 19 of WO 2017/175147)

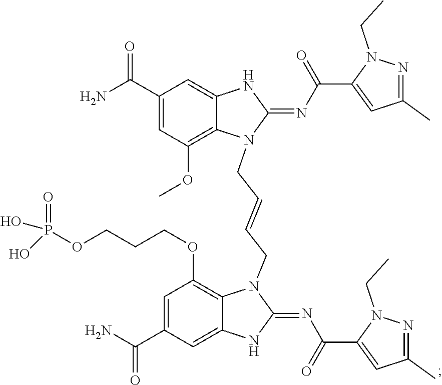 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCOP(=O)(O)O)c21]

(E)-4-((5-carbamoyl-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)butanoic acid (Example 52 of WO 2017/175147)

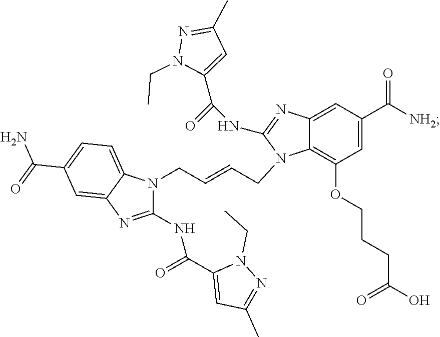 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCC(=O)O)c21]

(E)-1-(4-(5-carbamoyl-2-(1-ethyl-3-methyl-1H-pyrazole-5-carboxamido)-7-methoxy-1H-benzo[d]imidazol-1-yl)but-2-en-1-yl)-7-(3-(dimethylamino)propoxy)-2-(1-ethyl-3-methyl-1H-pyrazole-5-carboxamido)-1H-benzo[d]imidazole-5-carboxamide (Example 39 of WO 2017/175147)

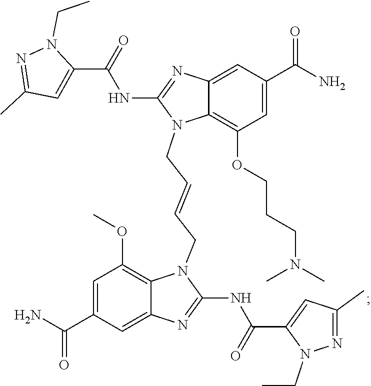

(E)-1-((E)-4-((E)-5-carbamoyl-2-((1-ethyl-3-methyl-1H-pyrazole-5-carbonyl)imino)-7-(3-(4-(2-hydroxyethyl)piperazin-1-yl)propoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide (Example 43 of WO 2017/175147)

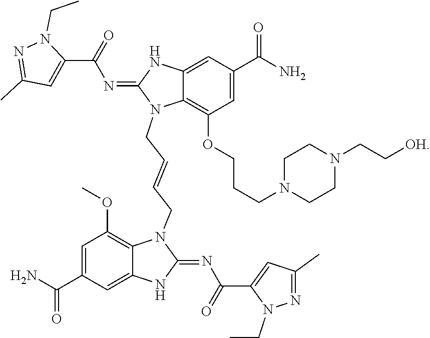 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(CCO)CC3)c21]

In one embodiment, the STING agonist is 3-(((Z)-6-Carbamoyl-3-((E)-4-((Z)-5-carbamoyl-2-((1-ethyl-3-methyl-1H-pyrazole-5-carbonyl)imino)-7-methoxy-2,3-dihydro-1H-benzo[d]imidazol-1-yl)but-2-en-1-yl)-2-((1-ethyl-3-methyl-1H-pyrazole-5-carbonyl)imino)-2,3-dihydro-1H-benzo[d]imidazol-4-yl)oxy)propyl dihydrogen phosphate

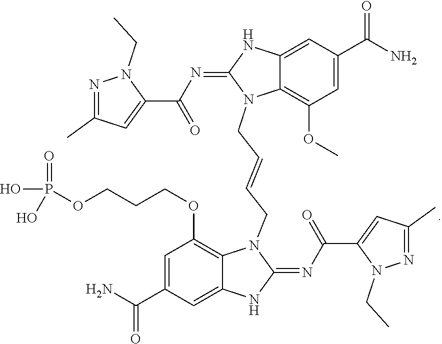 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCOP(=O)(O)O)c21]

In one embodiment, the STING agonist is (E)-3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl dihydrogen phosphate

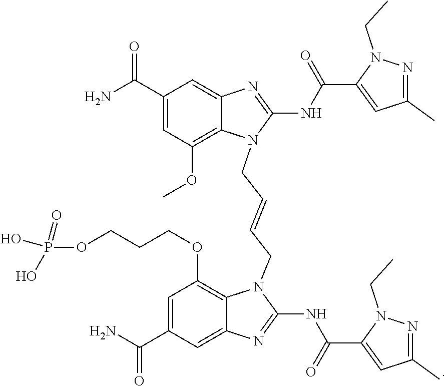 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCOP(=O)(O)O)c21]

In one embodiment, the STING agonist is by 3-(((E)-6-carbamoyl-3-((E)-4-((E)-5-carbamoyl-2-((1-ethyl-3-methyl-1H-pyrazole-5-carbonyl)imino)-7-methoxy-2,3-dihydro-1H-benzo[d]imidazol-1-yl)but-2-en-1-yl)-2-((1-ethyl-3-methyl-1H-pyrazole-5-carbonyl)imino)-2,3-dihydro-1H-benzo[d]imidazol-4-yl)oxy)propyl

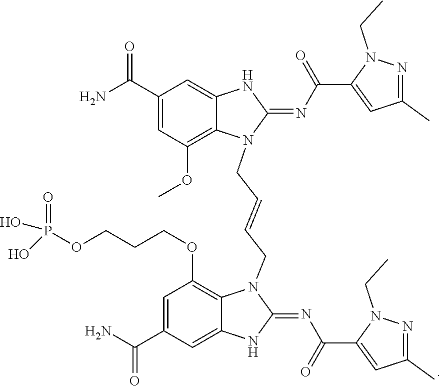 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCOP(=O)(O)O)c21]

Those skilled in the art will appreciate that STING agonists may contain one or more asymmetrically substituted atom(s), furthermore, geometric isomers of double bonds such as olefins and C═N double bonds may also be present in certain STING agonists, and all chiral, diastereomeric, racemic forms and all geometric isomeric forms of a structure are intended, unless the specific stereochemistry or isomeric form is specifically indicated. Additionally, certain compounds may exist as tautomers, and all such tautomers of a structure are intended unless the context requires otherwise.

Compounds may also contain ionisable groups and therefore may be presented as a pharmaceutically acceptable salt.

As used herein, “pharmaceutically acceptable salts” refer to derivatives of the disclosed compounds wherein the parent compound is modified by making pharmaceutically acceptable acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Lists of suitable salts are found in Remington's Pharmaceutical Sciences, 17th ed., Mack Publishing Company, Easton, P A, 1985, p. 1418, the disclosure of which is hereby incorporated by reference.

The phrase “pharmaceutically acceptable salt” is employed herein to refer to those salts which are, within the scope of sound medical judgment, suitable for use in a pharmaceutical context, without excessive toxicity, irritation, allergic response, or other problem or complication, commensurate with a reasonable benefit/risk ratio.

If provided in solution, STING agonists with ionisable groups may be in dissociated form. Pharmaceutically acceptable salts of STING agonists may be dissociated in solution.

If provided in solid form, the STING agonist may be in the form of a solvate, such as a hydrate.

A typical human dose of STING agonist may be 0.1 to 150 ug, especially 0.5 to 100 ug, such as 1 to 50 ug.

More than one STING agonist may be utilised, such as two. Typically only one STING agonist is utilised.

### Adjuvant Carriers

The STING agonist will typically be formulated with one or more carriers. Suitable carriers may include ISCOMs, liposomes and emulsions.

The term ‘liposome’ is well known in the art and defines a general category of vesicles which comprise one or more lipid bilayers surrounding an aqueous space. Liposomes thus consist of one or more lipid and/or phospholipid bilayers and can contain other molecules, such as proteins or carbohydrates, in their structure. Because both lipid and aqueous phases are present, liposomes can encapsulate or entrap water-soluble material, lipid-soluble material and/or amphiphilic compounds.

Liposome size may vary from 30 nm to several um depending on the phospholipid composition and the method used for their preparation. In the present invention, the liposome size will typically be in the range of 50 nm to 200 nm, especially 60 nm to 180 nm, such as 70 to 165 nm. Optimally, the liposomes should be stable and have a diameter of ˜100 nm to allow convenient sterilization by filtration.

The liposomes of use in the present invention suitably contain DOPC

The ratio of STING agonist:DOPC will typically be in the order of 1:50 to 1:10 (w/w), suitably between 1:25 to 1:15 (w/w), and preferably 1:22 to 1:18 (w/w), such as 1:20 (w/w).

Structural integrity of the liposomes may be assessed by methods such as dynamic light scattering (DLS) measuring the size (Z-average diameter, Zav) and polydispersity of the liposomes, or, by electron microscopy for analysis of the structure of the liposomes. In one embodiment the average particle size is between 95 and 120 nm, and/or, the polydispersity (PdI) index is not more than 0.35, in particular not more than 0.3, such as not more than 0.25. In one embodiment the average particle size is between 95 and 120 nm, and/or, the polydispersity (PdI) index is not more than 0.2. The average particle size may be 90 to 120 nm, and/or, the polydispersity (PdI) index is not more than 0.35, in particular not more than 0.3, such as not more than 0.25. In one embodiment the average particle size is 90 to 120 nm, and/or, the polydispersity (PdI) index is not more than 0.2.

Emulsion carriers will typically be an oil in water emulsion comprising a pharmaceutically acceptable metabolizable oil, such as squalene. Squalene, is a branched, unsaturated terpenoid ([(CH₃)₂C[═CHCH₂CH₂C(CH₃)]₂═CHCH₂—]₂; C₃₀H₅₀; 2,6,10,15,19,23-hexamethyl-2,6,10,14,18,22-tetracosahexaene; CAS Registry Number 7683-64-9). Squalene is readily available from commercial sources or may be obtained by methods known in the art. Squalene shows good biocompatibility and is readily metabolised.

Squalene emulsion adjuvants will typically have a submicron droplet size. Droplet sizes below 200 nm are beneficial in that they can facilitate sterilisation by filtration. There is evidence that droplet sizes in the 80 to 200 nm range are of particular interest for potency, manufacturing consistency and stability reasons. (Klucker, 2012; Shah, 2014; Shah, 2015; Shah, 2019). The squalene emulsion adjuvant may have an average droplet size of 50 to 200 nm, such as 80 to 200 nm, especially 120 to 180 nm, in particular 140 to 180 nm, such as about 160 nm.

Uniformity of droplet sizes is desirable. A polydispersity index (PdI) of greater than 0.7 indicates that the sample has a very broad size distribution and a reported value of 0 means that size variation is absent, although values smaller than 0.05 are rarely seen. Suitably the squalene emulsion adjuvant has a polydispersity of 0.5 or less, especially 0.3 or less, such as 0.2 or less.

The droplet size, as used herein, means the average diameter of oil droplets in an emulsion and can be determined in various ways e.g. using the techniques of dynamic light scattering and/or single-particle optical sensing, using an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan). See Light Scattering from Polymer Solutions and Nanoparticle Dispersions Schartl, 2007. Dynamic light scattering (DLS) is the preferred method by which droplet size is determined. The preferred method for defining the average droplet diameter is a Z-average i.e. the intensity-weighted mean hydrodynamic size of the ensemble collection of droplets measured by DLS. The Z-average is derived from cumulants analysis of the measured correlation curve, wherein a single particle size (droplet diameter) is assumed and a single exponential fit is applied to the autocorrelation function. Thus, references herein to average droplet size should be taken as an intensity-weighted average, and ideally the Z-average. PdI values are easily provided by the same instrumentation which measures average diameter.

In order to maintain a stable submicron emulsion, one or more emulsifying agents (i.e. surfactants) are generally required. Surfactant(s) will typically be metabolisable (biodegradable) and biocompatible, being suitable for use as a pharmaceutical. The surfactant can include ionic (cationic, anionic or zwitterionic) and/or non-ionic surfactants. The use of only non-ionic surfactants is often desirable, for example due to their pH independence. The invention can thus use surfactants including, but not limited to:
the polyoxyethylene sorbitan ester surfactants (commonly referred to as the Tweens or polysorbates), such as polysorbate 20 and polysorbate 80, especially polysorbate 80;
copolymers of ethylene oxide (EO), propylene oxide (PO), and/or butylene oxide (BO), sold under the DOWFAX™, Pluronic™ (e.g. F68, F127 or L121 grades) or Synperonic™ tradenames, such as linear EO/PO block copolymers, for example poloxamer 407, poloxamer 401 and poloxamer 188;
octoxynols, which can vary in the number of repeating ethoxy (oxy-1,2-ethanediyl) groups, with octoxynol-9 (Triton X-100, or t-octylphenoxypolyethoxyethanol) being of particular interest;
(octylphenoxy)polyethoxyethanol (IGEPAL CA-630/NP-40);
phospholipids such as phosphatidylcholine (lecithin);
polyoxyethylene fatty ethers derived from lauryl, cetyl, stearyl and oleyl alcohols (known as Brij surfactants), such as polyoxyethylene 4 lauryl ether (Brij 30, Emulgen 104P), polyoxyethylene-9-lauryl ether and polyoxyethylene 12 cetyl/stearyl ether (Eumulgin™ B1, cetereth-12 or polyoxyethylene cetostearyl ether);
sorbitan esters (commonly known as the Spans), such as sorbitan trioleate (Span 85), sorbitan monooleate (Span 80) and sorbitan monolaurate (Span 20);
or tocopherol derivative surfactants, such as alpha-tocopherol-polyethylene glycol succinate (TPGS).

Surfactants of particular interest include: poloxamer 401, poloxamer 188, polysorbate 80, sorbitan trioleate, sorbitan monooleate and polyoxyethylene 12 cetyl/stearyl ether either alone, in combination with each other or in combination with other surfactants. Especially of interest are polysorbate 80, sorbitan trioleate, sorbitan monooleate and polyoxyethylene 12 cetyl/stearyl ether either alone, or in combination with each other. A particular surfactant of interest is polysorbate 80. A particular combination of surfactants of interest is polysorbate 80 and sorbitan trioleate. A further combination of surfactants of interest is sorbitan monooleate and polyoxyethylene cetostearyl ether.

### Subjects

The present invention is generally intended for mammalian subjects, in particular human subjects. The subject may be a wild or domesticated animal. Mammalian subjects include for example cats, dogs, pigs, sheep, horses or cattle. In one embodiment of the invention, the subject is human.

The subject to be treated using the method of the invention may be of any age.

In one embodiment the subject is a human infant (up to 12 months of age). In one embodiment the subject is a human child (less than 18 years of age). In one embodiment the subject is an adult human (aged 18-59). In one embodiment the subject is an older human (aged 60 or greater).

Doses administered to younger children, such as less than 12 years of age, may be reduced relative to an equivalent adult dose, such as by 50%.

The methods of the invention may be intended for prophylaxis of infectious diseases, i.e. for administration to a subject which is not infected with a pathogen. In other embodiments the methods of the invention may be intended for treatment, e.g. for the treatment of infectious diseases, i.e. for administration to a subject which is infected with a pathogen.

### Formulation and Administration

The carrier-formulated mRNA and adjuvant comprising a STING agonist may be administered as a formulation containing the carrier-formulated mRNA and adjuvant comprising a STING agonist (‘co-formulation’ or ‘co-formulated’). Alternatively the carrier-formulated mRNA and adjuvant comprising a STING agonist may be administered as two or more formulations, for example, a first formulation containing the carrier-formulated mRNA and a second formulation containing the adjuvant comprising a STING agonist (‘separate formulation’ or ‘separately formulated’). Consequently, it will be appreciated that a range of formulation possibilities exist. A reasonable balance is desirable between practical considerations such as: compatibility of components for co-formulation; manufacture, storage and distribution of a plurality of single vs multiple component formulations; and the need for multiple administrations in the case of separate formulation.

When separately formulated, the carrier-formulated mRNA and STING agonist may be administered through the same or different routes, to the same or different locations, and at the same or different times.

The carrier-formulated mRNA and STING agonist may be administered via various suitable routes, including parenteral, such as intramuscular or subcutaneous administration. The carrier-formulated mRNA and STING agonist may be administered via different routes. Suitably the carrier-formulated mRNA and STING agonist are administered via the same route, in particular intramuscularly.

When administered as separate formulations, the carrier-formulated mRNA and STING agonist are desirably administered to locations with sufficient spatial proximity such that the adjuvant effect is adequately maintained. For example, spatial proximity is sufficient to maintain at least 50%, especially at least 75% and in particular at least 90% of the adjuvant effect seen with administration to the same location. The adjuvant effect seen with administration to the same location is defined as the level of increase observed as a result of administration of carrier-formulated mRNA and STING agonist to the same location compared with administration of carrier-formulated mRNA alone. The carrier-formulated mRNA and STING agonist are desirably administered to a location draining to the same lymph node, such as to the same limb, in particular to the same muscle.

Suitably carrier-formulated mRNA and STING agonist are administered intramuscularly to the same muscle. In certain embodiments, the carrier-formulated mRNA and STING agonist are administered to the same location.

The spatial separation of administration locations may be at least 5 mm, such as at least 1 cm.

The spatial separation of administration locations may be less than 10 cm, such as less than 5 cm apart.

When administered as separate formulations, the carrier-formulated mRNA and STING agonist are desirably administered with sufficient temporal proximity such that the adjuvant effect is adequately maintained. For example, temporal proximity is sufficient to maintain at least 50%, especially at least 75% and in particular at least 90% of the adjuvant effect seen with administration at the same time. The adjuvant effect seen with administration at the same time is defined as the level of increase observed as a result of administration of carrier-formulated mRNA and STING agonist at (essentially) the same time compared with administration of carrier-formulated mRNA without STING agonist.

When administered as separate formulations, carrier-formulated mRNA and STING agonist may be administered within 84 hours, such as within 60 hours, especially within 36 hours, in particular within 24 hours, for example within 12 hours. Suitably the carrier-formulated mRNA and STING agonist are administered within 6 hours, especially within 2 hours, in particular within 1 hour, such as within 30 minutes and especially within 15 minutes (e.g. within 5 minutes). In one embodiment the carrier-formulated mRNA and STING agonist are administered within 12 hours. In a further embodiment the carrier-formulated mRNA and STING agonist are administered within 12 to 36 hours. In another embodiment the carrier-formulated mRNA and STING agonist are administered within 36 to 84 hours.

The delay between administration of the carrier-formulated mRNA and STING agonist may be at least 5 seconds, such as 10 seconds, and in particular at least 30 seconds.

When administered as separate formulations, if the carrier-formulated mRNA and STING agonist are administered with a delay, the carrier-formulated mRNA may be administered first and the STING agonist administered second. Alternatively, the STING agonist is administered first and the carrier-formulated mRNA is administered second. Appropriate temporal proximity may depend on the order of administration.

Desirably, the carrier-formulated mRNA and STING agonist are administered without intentional delay (accounting for the practicalities of multiple administrations).

In addition to co-formulated or separately formulated presentations of carrier-formulated mRNA and STING agonist for direct administration, the carrier-formulated mRNA and STING agonist may initially be provided in various forms which facilitate manufacture, storage and distribution. For example, certain components may have limited stability in liquid form, certain components may not be amendable to drying, certain components may be incompatible when mixed (either on a short- or long-term basis). Independent of whether carrier-formulated mRNA and STING agonist are co-formulated at administration, they may be provided in separate containers the contents of at least some of which are subsequently combined. The skilled person will appreciate that many possibilities exist, although it is generally desirable to have a limited number of containers and limited number of required steps to prepare the final co-formulation or separate formulations for administration.

Carrier-formulated mRNA may be provided in liquid or dry (e.g. lyophilised) form. The preferred form will depend on factors such as the precise nature of the carrier-formulated mRNA, e.g. if the carrier-formulated mRNA is amenable to drying, or other components which may be present. The carrier-formulated mRNA is typically provided in liquid form.

The STING agonist may be provided in liquid or dry form. The preferred form will depend on the precise nature of the STING agonist and any associated carrier, e.g. if capable of reconstitution from dry form, and any other components present. The STING agonist is typically provided in liquid form.

The invention provides a composition comprising carrier-formulated mRNA encoding an antigen and adjuvant comprising a STING agonist. Typically, carrier-formulated mRNA encoding an antigen and adjuvant comprising a STING agonist are provided as a liquid co-formulation. A liquid co-formulation enables convenient administration at the point of use.

In other embodiments the carrier-formulated mRNA encoding an antigen and adjuvant comprising a STING agonist are provided as a dry co-formulation, the dry co-formulation being reconstituted prior to administration. A dry co-formulation, where the components of the formulation are amendable to such presentation, may improve stability and thereby facilitate longer storage.

The carrier-formulated mRNA encoding an antigen and adjuvant comprising a STING agonist may be provided in separate containers. The invention therefore provides carrier-formulated mRNA encoding an antigen for use with an adjuvant comprising a STING agonist.

Also provided is an adjuvant comprising a STING agonist for use with carrier-formulated mRNA encoding an antigen. Further provided is a kit comprising:
(i) a first container comprising carrier-formulated mRNA encoding an antigen; and
(ii) a second container comprising adjuvant comprising a STING agonist.

The carrier-formulated mRNA encoding an antigen may be in liquid form and the adjuvant comprising a STING agonist may be in liquid form. In such cases the contents of the first and second containers may be intended for combination to provide a co-formulation for administration. Alternatively, the contents of each container may be intended for separate administration as the first and second formulations.

The carrier-formulated mRNA encoding an antigen may be in dry form and the adjuvant comprising a STING agonist may be in liquid form. In such cases the contents of the first and second containers may be intended for combination to provide a co-formulation for administration. Alternatively, the carrier-formulated mRNA encoding an antigen may be intended to be reconstituted prior to the contents of each container being used for separate administration as the first and second formulations.

The adjuvant comprising a STING agonist may be in dry form and the carrier-formulated mRNA encoding an antigen may be in liquid form. In such cases the contents of the first and second containers may be intended for combination to provide a co-formulation for administration. Alternatively, the adjuvant comprising a STING agonist may be intended to be reconstituted prior to the contents of each container being used for separate administration as the first and second formulations.

The carrier-formulated mRNA may be in dry form and the adjuvant comprising a STING agonist may be in dry form. In such cases the contents of the first and second containers may be intended for reconstitution and combination to provide a co-formulation for administration. Reconstitution may occur separately before combination, or the contents of one container may be reconstituted and then used to reconstitute the contents of the other container. Alternatively, the contents of the first and second containers may be intended for reconstitution prior to the contents of each container being used for separate administration as the first and second formulations.

If appropriate to the circumstances, liquid forms may be stored frozen.

The precise composition of liquid used for reconstitution will depend on both the contents of a container being reconstituted and the subsequent use of the reconstituted contents e.g. if they are intended for administration directly or may be combined with other components prior to administration. A composition (such as those containing carrier-formulated mRNA encoding an antigen or adjuvant comprising a STING agonist) intended for combination with other compositions prior to administration need not itself have a physiologically acceptable pH or a physiologically acceptable tonicity; a formulation intended for administration should have a physiologically acceptable pH and should have a physiologically acceptable osmolality.

The pH of a liquid preparation is adjusted in view of the components of the composition and necessary suitability for administration to the human subject. The pH of a formulation is generally at least 4, especially at least 5, in particular at least 5.5 such as at least 6. The pH of a formulation is generally 9 or less, especially 8.5 or less, in particular 8 or less, such as 7.5 or less. The pH of a formulation may be 4 to 9, especially 5 to 8.5, in particular 5.5 to 8, such as 6.5 to 7.4 (e.g. 6.5 to 7.1).

For parenteral administration, solutions should have a physiologically acceptable osmolality to avoid excessive cell distortion or lysis. A physiologically acceptable osmolality will generally mean that solutions will have an osmolality which is approximately isotonic or mildly hypertonic. Suitably the formulations for administration will have an osmolality of 250 to 750 mOsm/kg, especially 250 to 550 mOsm/kg, in particular 270 to 500 mOsm/kg, such as 270 to 400 mOsm/kg. Osmolality may be measured according to techniques known in the art, such as by the use of a commercially available osmometer, for example the Advanced® Model 2020 available from Advanced Instruments Inc. (USA).

Liquids used for reconstitution will be substantially aqueous, such as water for injection, phosphate buffered saline and the like. As mentioned above, the requirement for buffer and/or tonicity modifying agents will depend on both the contents of the container being reconstituted and the subsequent use of the reconstituted contents. Buffers may be selected from acetate, citrate, histidine, maleate, phosphate, succinate, tartrate and TRIS. The buffer may be a phosphate buffer such as Na/Na₂PO₄, Na/K₂PO₄ or K/K₂PO₄.

It will be appreciated that some of the components used may form salts under appropriate conditions, therefore such components may be present as a salt, in particular a pharmaceutically acceptable salt.

Suitably, the formulations used in the present invention have a dose volume of between 0.05 ml and 1 ml, such as between 0.1 and 0.6 ml, in particular a dose volume of 0.45 to 0.55 ml, such as 0.5 ml. The volumes of the compositions used may depend on the subject, delivery route and location, with smaller doses being given by the intradermal route or if the carrier-formulated mRNA and STING agonist are delivered separately to the same location. A typical human dose for administration through routes such as intramuscular, is in the region of 200 ul to 750 ml, such as 400 to 600 ul, in particular about 500 ul, such as 500 ul.

If two liquids are intended to be combined, for example for co-formulation if the carrier-formulated mRNA is in liquid form and the adjuvant comprising a STING agonist is in liquid form, the volume of each liquid may be the same or different. Volumes for combination will typically be in the range of 10:1 to 1:10, such as 2:1 to 1:2. Suitably the volume of each liquid will be substantially the same, such as the same. For example a 250 ul volume of carrier-formulated mRNA in liquid form may be combined with a 250 ul volume adjuvant comprising a STING agonist in liquid form to provide a co-formulation dose with a 500 ul volume, each of the carrier-formulated mRNA and adjuvant comprising a STING agonist being diluted 2-fold during the combination.

Adjuvants comprising a STING agonist may therefore be prepared as a concentrate with the expectation of dilution by a liquid carrier-formulated mRNA containing composition prior to administration. For example, adjuvant comprising a STING agonist may be prepared at double-strength with the expectation of dilution by an equal volume of carrier-formulated mRNA containing composition prior to administration.

Carrier-formulated mRNA and adjuvant comprising a STING agonist, whether intended for co-formulation or separate formulation, may be provided in the form of various physical containers such as vials or pre-filled syringes.

In some embodiments the carrier-formulated mRNA, adjuvant comprising a STING agonist or kit comprising carrier-formulated mRNA and adjuvant comprising a STING agonist is provided in the form of a single dose. In other embodiments the carrier-formulated mRNA, adjuvant comprising a STING agonist or kit comprising carrier-formulated mRNA and adjuvant comprising a STING agonist is provided in multidose form such containing 2, 5 or 10 doses. Multidose forms, such as those comprising 10 doses, may be provided in the form of a plurality of containers with single doses of one part (e.g. the carrier-formulated mRNA) and a single container with multiple doses of the second part (e.g. adjuvant comprising a STING agonist) or may be provided in the form of a single container with multiple doses of one part (carrier-formulated mRNA) and a single container with multiple doses of the second part (e.g. adjuvant comprising a STING agonist).

It is common where liquids are to be transferred between containers, such as from a vial to a syringe, to provide ‘an overage’ which ensures that the full volume required can be conveniently transferred. The level of overage required will depend on the circumstances but excessive overage should be avoided to reduce wastage and insufficient overage may cause practical difficulties. Overages may be of the order of 20 to 100 ul per dose, such as 30 ul or 50 ul. For example, a typical 10 dose container of doubly concentrated adjuvant (250 ul per dose) may contain around 2.85 to 3.25 ml of adjuvant.

Stabilisers may be present. Stabilisers may be of particular relevance where multidose containers are provided as doses of the final formulation(s) may be administered to subjects over a period of time.

Carrier-formulated mRNA and adjuvant comprising a STING agonist in liquid form may be provided in the form of a multichamber syringe. The use of multi-chamber syringes provides a convenient method for the separate sequential administration of the carrier-formulated mRNA and adjuvant comprising a STING agonist. Multi-chamber syringes may be configured to provide concurrent but separate delivery of the carrier-formulated mRNA and adjuvant comprising a STING agonist, or they may be configured to provide sequential delivery (in either order).

In other configurations of multichambered syringes, the carrier-formulated mRNA may be provided in dry form (e.g., freeze-dried) in one chamber and reconstituted by the adjuvant comprising a STING agonist contained in the other chamber before administration.

Examples of multi-chamber syringes may be found in disclosures such as WO2016/172396, although a range of other configurations are possible.

Formulations are preferably sterile.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the carrier-formulated mRNA and adjuvant comprising a STING agonist may therefore be part of a multi-dose administration regime. For example, the carrier-formulated mRNA and adjuvant comprising a STING agonist may be provided as a priming dose in a multidose regime, especially a two- or three-dose regime, in particular a two-dose regime. The carrier-formulated mRNA and adjuvant comprising a STING agonist may be provided as a boosting dose in a multidose regime, especially a two- or three-dose regime, such as a two-dose regime.

Priming and boosting doses may be homologous or heterologous. Consequently, the carrier-formulated mRNA and adjuvant comprising a STING agonist may be provided as a priming dose and boosting dose(s) in a homologous multidose regime, especially a two- or three-dose regime, in particular a two-dose regime. Alternatively, the carrier-formulated mRNA and adjuvant comprising a STING agonist may be provided as a priming dose or boosting dose in a heterologous multidose regime, especially a two- or three-dose regime, in particular a two-dose regime, and the boosting dose(s) may be different (e.g. a different carrier-formulated mRNA; or an alternative antigen presentation such as protein or virally vectored antigen—with or without adjuvant, such as adjuvant comprising a STING agonist).

The time between doses may be two weeks to six months, such as three weeks to three months. Periodic longer-term booster doses may also be provided, such as every 2 to 10 years.

The adjuvant comprising a STING agonist may be administered to a subject separately from carrier-formulated mRNA, or the adjuvant may be combined, either during manufacturing or extemporaneously, with carrier-formulated mRNA to provide an immunogenic composition for combined administration.

Consequently, there is provided a method for the preparation of an immunogenic composition comprising an adjuvant comprising a STING agonist and carrier-formulated mRNA encoding an antigen, said method comprising the steps of:
(i) preparing an adjuvant comprising a STING agonist;
(ii) mixing the adjuvant comprising a STING agonist with carrier-formulated mRNA encoding an antigen.

Also provided is a method for the preparation of an immunogenic composition comprising an adjuvant comprising a STING agonist and carrier-formulated mRNA encoding an antigen, said method comprising the steps of:
(i) preparing carrier-formulated mRNA encoding an antigen;
(ii) mixing the carrier-formulated mRNA encoding an antigen with adjuvant comprising a STING agonist.

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in or “approximately” in relation to a numerical value x is optional and means, for example, x±10% of the given figure, such as x±5% of the given figure, in particular the given figure.

As used herein, the singular forms “a,” “an” and “the” include plural references unless the content clearly dictates otherwise.

As used herein, ng refers to nanograms, ug or pg refers to micrograms, mg refers to milligrams, mL or ml refers to milliliter, and mM refers to millimolar. Similar terms, such as um, are to be construed accordingly.

Unless specifically stated, a process comprising a step of mixing two or more components does not require any specific order of mixing. Thus components can be mixed in any order. Where there are three components then two components can be combined with each other, and then the combination may be combined with the third component, etc.

### Disclaimers

The following disclaimers may optionally be applied alone, or in combination, to any embodiment of the invention.

Suitably the antigen is not a Zika virus pre-M-E antigen (prME), or an immunogenic fragment or variant thereof. Desirably the antigen is not a Zika virus antigen.

Suitably the antigen is not a respiratory syncytial virus (RSV) F antigen. Desirably the antigen is not an RSV antigen.

Suitably the antigen is not a human immunodeficiency virus (HIV) gp140 antigen.

Desirably the antigen is not an HIV antigen.

Suitably the antigen is not a Leishmania antigen.

Suitably the antigen is not a glycoprotein.

Suitably the mRNA carrier is not a LION. Desirably the mRNA carrier does not comprise iron oxide particles, in particular the carrier does not comprise inorganic oxide particles.

Suitably administration of the carrier-formulated mRNA in conjugation with the adjuvant comprising a STING agonist does not significantly alter (such as less than +/−25% change, especially less than +/−10% change and in particular less than +/−5% change) the level of antigen expression observed in the absence of adjuvant comprising a STING agonist.

### EXAMPLES

### Example 1—Immunogenicity Evaluation of SAM Administered with Adjuvant Comprising a STING Agonist in Mice

### Materials and Methods

### Mouse Immunisation, Vector Production and Vaccination Scheme

Female CB6F1 inbred mice aged 6-8 weeks old were randomly assigned to study groups (n=8 gr1-10 (groups 1-10) and n=4/gr11 (group 11)) and kept under pathogen-free conditions. Mice (gr1-14) were intramuscularly (i.m.) immunized, in the gastrocnemius muscle, on days 0 and 28 with 0.1ug of LNP-formulated SAM (25 uL volume) comprising a heterodimer of the HSV-2 gE and gI proteins (LNP/SAM-gE_P317R/gI) and were injected, in the same muscle, with different doses of STING agonist (0.25, 0.74 or 2.22ug) either at the same time (day+0) or 1 or 3 days after each of the two SAM immunizations. As a positive control for vaccine response, mice immunized with 0.lug of LNP/SAM-gE_P317R/gI vaccine were i.m. injected, at the same time as the immunization, with a saline solution (NaCl 150 mM) instead of adjuvant. Finally, as negative control, mice were i.m injected with only 50 uL of a saline solution (NaCl 150 mM), following the same schedule of immunization (see Table 1 for details of each group).

The STING agonist used was 3-(((E)-6-carbamoyl-3-((E)-4-((E)-5-carbamoyl-2-((1-ethyl-3-methyl-1H-pyrazole-5-carbonyl)imino)-7-methoxy-2,3-dihydro-1H-benzo[d]imidazol-1-yl)but-2-en-1-yl)-2-((1-ethyl-3-methyl-1H-pyrazole-5-carbonyl)imino)-2,3-dihydro-1H-benzo[d]imidazol-4-yl)oxy)propyl

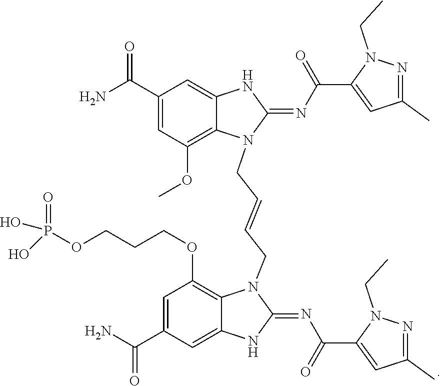 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCOP(=O)(O)O)c21]

TABLE 1
Summary of the study design and formulations tested

| | | | | Adjuvant | |
|---|---|---|---|---|---|
| | | | Vaccine | dose | |
| | | | immuni- | and | Volume |
| | Number | | zation | immuni- | and |
| | of | Vaccine | schedule | zation | route of |
| Group | animals | name & dose | (Days) | schedule | injection |
| 1 | 8 | LNP/SAM-gE_ | Days | 2.22 μg | i.m. |
| | | P317R/gl | 0 & 28 | Day + 0 | 25 uL each |
| | | (0.1 ug) | | | |
| 2 | 8 | LNP/SAM-gE_ | Days | 2.22 μg | i.m. |
| | | P317R/gl | 0 & 28 | Day + 1 | 25 uL each |
| | | (0.1 ug) | | | |
| 3 | 8 | LNP/SAM-gE_ | Days | 2.22μg | i.m. |
| | | P317R/gl | 0 & 28 | Day + 3 | 25 uL each |
| | | (0.1 ug) | | | |
| 4 | 8 | LNP/SAM-gE_ | Days | 0.74 μg | i.m. |
| | | P317R/gl | 0 & 28 | Day + 0 | 25 uL each |
| | | (0.1 ug) | | | |
| 5 | 8 | LNP/SAM-gE_ | Days | 0.74 μg | i.m. |
| | | P317R/gl | 0 & 28 | Day + 1 | 25 uL each |
| | | (0.1 ug) | | | |
| 6 | 8 | LNP/SAM-gE_ | Days | 0.74 μg | i.m. |
| | | P317R/gl | 0 & 28 | Day + 3 | 25 uL each |
| | | (0.1 ug) | | | |
| 7 | 8 | LNP/SAM-gE_ | Days | 0.25 μg | i.m. |
| | | P317R/gl | 0 & 28 | Day + 0 | 25 uL each |
| | | (0.1 ug) | | | |
| 8 | 8 | LNP/SAM-gE_ | Days | 0.25 μg | i.m. |
| | | P317R/gl | 0 & 28 | Day + 1 | 25 uL each |
| | | (0.1 ug) | | | |
| 9 | 8 | LNP/SAM-gE_ | Days | 0.25 μg | i.m. |
| | | P317R/gl | 0 & 28 | Day + 3 | 25 uL each |
| | | (0.1 ug) | | | |
| 10 | 8 | LNP/SAM-gE_ | Days | NaCl 150 mM | i.m. |
| | | P317R/gl | 0 & 28 | Day + 0 | 25 uL each |
| | | (0.1 ug) | | | |
| 11 | 4 | NaCl 150 mM | Days | NaCl 150 mM | i.m. |
| | | | 0 & 28 | Day + 0 | 25 uL each |

Serum samples were collected at days 24 & 49 post prime immunization (24PI, 21 PII) to measure anti-HSV-2 gE, anti-HSV-2 gI and anti-HSV-1 gE/gI IgG antibody responses. The function of antibodies was investigated only after the second immunization at day 49 (21PII). Spleens were also collected 21 days post second immunization (21PII) to evaluate vaccine-specific CD4+/CD8+ T cell responses. The study was split across two independent experiments—Experiment A and Experiment B.

The SAM pTC83R_P989 plasmid (pRIT17148, HSV-2_gE_P317R_IRES_gI SAM) was designed as a bicistronic vector where i) gE expression is driven by a single promoter (subgenomic promoter) and ii) gI expression was driven by an Internal Ribosome Entry Site from enterovirus 71 (IRES EV71). Moreover, in order to knock down Fc binding activity, mutation P317R was introduced in the gE sequence. HSV-2 gE/gI sequences were codon optimized for expression in humans. For the gE sequence, nucleotides (nt) from 1 to 1257 were included in the construction and for gI sequence, nt from 1 to 768. The nucleotide sequence of the HSV-2_gE_P317R_IRES_gI insert is provided in SEQ ID NO: 5. The insert comprises ApaI, NotI, Tth111I and BstBI restriction sites and a double stop codon.

The nucleotide sequence of the parent SAM pTC83R_P989 plasmid (comprising SAM backbone sequences which are not incorporated into the SAM gE_P317R_IRES_gI transcript) is provided in SEQ ID NO: 6 and comprises the functional elements shown in Table 2. The polynucleotide sequence of SAM gE_P317R_IRES_gI is provided in SEQ ID NO: 7. The SAM pTC83R_P989 transcript theoretical RNA sequence is provided in SEQ ID NO: 8. This sequence is 5′-capped with 7-methylguanosine (Cap 0). The resultant polynucleotide is referred to herein as SAM-gE_P317R/gI. When administered to mice in LNP, this is referred to herein as LNP/SAM-gE_P317R/gI. This polynucleotide encodes the gE P317R and gI amino acid sequences which are provided in SEQ ID NO: 9 and 10, respectively.

TABLE 2
SAM pTC83R_P989 plasmid functional elements

| Position | Name |
|---|---|
| 10482 . . . 12775 | E. coli vector backbone |
| 12758 . . . 12774 | T7 promoter |
| 12775 | Transcription start site |
| 12775 . . . 10521 | SAM HSV-2 gE/gl primary transcript |
| 1 . . . 44 | 5′UTR (5′-untranslated region) |
| 45 . . . 7526 | Non-structural polyprotein nsP1234 |
| 7513 . . . 7536 | Subgenomic promoter |
| 7501 . . . 7506 | ApaI upstream cloning site |
| 7541 . . . 7549 | Tth111I site |
| 7537 . . . 7561 | UTR upstream of the target gene′s coding sequence |
| 7562 . . . 9136 | Coding sequence of the target gene |
| | (gE P317R_IRES_gl) |
| 8922 . . . 8927 | BstBI site |
| 10340 . . . 10364 | Synthetic vector polylinker with NotI |
| | downstream cloning site |
| 10365 . . . 10481 | 3′UTR (3′-untranslated region) |
| 10482 . . . 10522 | Synthetic 3′-polyA sequence |
| Complement: | BspQI site |
| 10523-10530 | |

SAM-LNP was prepared by rapidly mixing ethanolic solutions of lipids with aqueous buffers that contain SAM. The rapid mixing resulted in a supersaturation of hydrophobic components that have ionically paired with SAM. The SAM/lipid complexes condense and precipitate through nucleation mediated precipitation, yielding small and narrowly disperse nanoparticles. Following this mixing step, the lipid nanoparticles matured to entrap the RNA and then were transferred to a final Tris/sucrose storage buffer through a buffer exchange step. The LNP solutions were then characterized for size, lipid content, RNA entrapment, final SAM concentration, and in vitro potency. The LNP solutions comprised 20 mM Tris, 5 mM NaCl, 7.5% sucrose, and had a pH of 8.

### Detection of Total Anti-HSV-2 gE & gI IqG Antibodies by ELISA

Quantification of the total HSV-2 gE or gI-specific IgG antibodies was performed using indirect ELISA. Recombinant HSV-2 gE (˜51 kDa) or HSV-2 gI proteins (˜46 kDa) were used as coating antigens.

Polystyrene 96-well ELISA plates were coated with 100 uL/well of antigen diluted at a concentration of 2 ug/mL (HSV-2 gE) and 1 ug/mL (HSV-2 gI) in carbonate/bicarbonate 50 mM pH 9.5 buffer and incubated overnight at 4° C. After incubation, the coating solution was removed and the plates were blocked with 200 uL/well of skimmed milk media additive 10% diluted in PBS (blocking buffer) for 1 h at 37° C. The blocking solution was removed and or three-fold sera dilutions (in PBS+0.1% Tween20+1% BSA buffer) were added to the coated plates and incubated for 1h at 37° C. The plates were washed four times with PBS 0.1% Tween20 (washing buffer) and peroxidase conjugated goat anti-mouse IgG (H+L) was used as secondary antibody. One hundred microliters per well of the secondary antibody diluted at a concentration of 1:500 in PBS+0.1% Tween20+1% BSA buffer was added to each well and the plates were incubated for 45 min at 37° C.

The plates were then washed four times with washing buffer and 2 times with deionised water and incubated for 15 min at RT (room temperature) with 100 uL/well of a solution of 75% single-component TMB peroxidase ELISA substrate diluted in sodium citrate 0.1M pH 5.5 buffer. Enzymatic color development was stopped with 100 uL of 0.4N sulfuric acid 1M (H₂SO₄) per well and the plates were read at an absorbance of 450/620 nm using an ELISA reader. Optical densities (OD) were captured and analysed. A standard curve was generated by applying a 4-parameter logistic regression fit to a reference standard. Antibody titer in the samples was calculated by interpolation of the standard curve. The antibody titer of the samples was obtained by averaging the values from dilutions that fell within the 20-80% dynamic range of the standard curve. All ELISA titers were normalized using a standard reference to allow titer comparisons.

Detection of Total Anti-HSV-1 gE/gI-Specific IqG Antibodies by ELISA

Quantification of the total HSV-1 gE/gI-specific IgG antibodies was performed using indirect ELISA. Recombinant gE/gI heterodimer protein from HSV-1 was used as coating antigens.

Polystyrene 96-well ELISA plates were coated with 100 uL/well of antigen diluted at a concentration of 2 ug/mL in carbonate/bicarbonate 50 mM pH 9.5 buffer and incubated overnight at 4° C. After incubation, the coating solution was removed and the plates were blocked with 200 uL/well of skimmed milk media additive 10% diluted in PBS (blocking buffer) for 1 h at 37° C. The blocking solution was removed and three-fold sera dilutions (in PBS+0.1% Tween20+1% BSA buffer) were added to the coated plates and incubated for 1h at 37° C. The plates were washed four times with PBS 0.1% Tween20 (washing buffer) and peroxidase conjugated goat anti-mouse IgG (H+L) was used as secondary antibody. One hundred microliters per well of the secondary antibody diluted at a concentration of 1:500 in PBS+0.1% Tween20+1% BSA buffer was added to each well and the plates were incubated for 45 min at 37° C.

The plates were then washed four times with washing buffer and 2 times with deionised water and incubated for 15 min at RT (room temperature) with 100 uL/well of a solution of 75% single-component TMB peroxidase ELISA substrate diluted in sodium citrate 0.1M pH5.5 buffer. Enzymatic color development was stopped with 100 uL of 0.4N sulfuric acid 1M (H₂SO₄) per well and the plates were read at an absorbance of 450/620 nm using an ELISA reader. Optical densities (OD) were captured and analysed. A standard curve was generated by applying a 4-parameter logistic regression fit to a reference standard. Antibody titer in the samples was calculated by interpolation of the standard curve. The antibody titer of the samples was obtained by averaging the values from dilutions that fell within the 20-80% dynamic range of the standard curve. All ELISA titers were normalized using a standard reference to allow titer comparisons.

### Evaluation of Anti-HSV-2 g and gI CD4+/CD8+ T Cell Responses by Intracellular Cytokine Staining (ICS)

The frequencies of vaccine-specific CD4+ and CD8+ T-cells producing IL-2 and/or IFN-γ and/or TNF-α and/or IL-13 and/or IL-17 cytokines were evaluated in splenocytes collected 21 days after second immunization after ex-vivo stimulation with HSV-2 gE or gI peptide pools.

### Isolation of Splenocytes

Spleens were collected from individual mice 21 days after second immunization and placed in RPMI 1640 medium supplemented with RPMI additives (glutamine, penicillin/streptomycin, sodium pyruvate, non-essential amino-acids & 2-mercaptoethanol) (=RPMI/additives). Cell suspensions were prepared from each spleen using a tissue grinder. The splenic cell suspensions were filtered and then the filter was rinsed with 35 mL of cold PBS-EDTA. After centrifugation (335 g, 10 min at 4° C.), cells were resuspended in 5 mL of cold PBS-EDTA. A second washing step was performed, as previously described, and the cells were finally resuspended in 2 mL of RPMI/additives supplemented with 5% FCS. Cell suspensions were then diluted 20× (10 uL) in PBS buffer (190 uL) for cell counting. After counting, cells were centrifuged (335 g, 10 min at RT) and resuspended at 10⁷ cells/mL in RPMI/additives supplemented with 5% FCS.

### Cell Preparation

Fresh splenocytes were seeded in round bottom 96-well plates at 10⁶ cells/well (100 uL). The cells were then stimulated for 6 hours (37° C., 5% CO₂) with anti-CD28 and anti-CD49d antibodies at 1ug/mL per well, containing 100 uL of either:
15 mer overlapping peptide pool covering the sequences of gE protein from HSV-2 (1ug/mL per peptide per well).
15 mer overlapping peptide pool covering the sequences of gI protein from HSV-2 (1ug/mL per peptide per well).
15 mer overlapping peptide pool covering the sequences of human p-actin protein (1ug/mL per peptide per well) (irrelevant stimulation).
RPMI/additives medium (as negative control of the assay).
PMA—ionomycin solution at working concentrations of 0.25 ug/mL and 2.5 ug/mL respectively (as positive control of the assay).

After 2 hours of ex vivo stimulation, brefeldin A protein transport inhibitor diluted 1/200 in RPMI/additives supplemented with 5% FCS was added for 4 additional hours to inhibit cytokine secretion. Plates were then transferred at 4° C. for overnight incubation.

### Intracellular Cytokine Staining

After overnight incubation at 4° C., cells were transferred to V-bottom 96-well plates, centrifuged (189 g, 5 min at 4° C.) and washed with 250 uL of cold PBS+1% FCS (flow buffer). After a second centrifugation (189 g, 5 min at 4° C.), cells were resuspended to block unspecific antibody binding (10 min at 4° C.) in 50 uL of flow buffer containing anti-CD16/32 antibodies diluted 1/50. Then, 50 uL flow buffer containing mouse anti-CD4-A700 antibodies, anti-CD8-PerCp-Cy5.5 antibodies, anti-CD26L-BV786 antibodies, anti-CD127-BV421 antibodies and fixable yellow dead cell stain was added for 30 min in darkness at 4° C. After incubation, 100 uL of flow buffer was added into each well and cells were then centrifuged (189 g for 5 min at 4° C.). A second washing step was performed with 200 uL of flow buffer and after centrifugation, cells were fixed and permeabilized by adding 200 uL of fixation solution for 20 min at 4° C. in darkness. After plate centrifugation (500 g for 5 min at 4° C.), cells were washed with 200 uL of cell wash buffer, centrifuged (500 g for 5 min at 4° C.) and resuspended in 50 uL of cell wash buffer containing mouse anti-IL2-FITC, anti- IFN-γ-APC, anti-TNF-α-PE, anti-IL-13-PE-Cy7 and anti-IL-17-BV605 antibodies, for 1 hour at 4° C. in darkness. After incubation, 100 uL of flow buffer was added into each well and cells were then finally washed with 200 uL of cell wash buffer (centrifugation 500 g for 5 min at 4° C.) and resuspended in 220 uL PBS.

### Cell Acquisition and Analysis

Stained cells were acquired by flow cytometry and analyzed. Live cells were identified with staining and then lymphocytes were isolated based on forward/side scatter lights (FSC/SSC) gating. The acquisition was performed on ˜20,000 CD4+/CD8+ T-cell events. The percentages of IFN-γ^{+/-}, IL-2^{+/-}, TNF-α^{+/-}, IL-13^{+/-} and IL-17^{+/-} producing cells were calculated on CD4+ and CD8+ T cell populations. For each sample, unspecific signal detected after medium stimulation was removed from the specific signal detected after peptide pool stimulation.

Competitive ELISA to Evaluate the Ability of Vaccine-Specific Antibodies to Decrease Human IgG Fc Binding by HSV-2 gE/gI Protein

The ability of polyclonal sera (pAbs) collected in different groups of mice to decrease in-vitro hIgG Fc binding by recombinant HSV-2 gE/gI protein was investigated by competitive ELISA. Recombinant HSV-2 gE/gI protein was used as coating antigen.

Polystyrene 96-well ELISA plates were coated with 50 uL/well of HSV-2 gE/gI protein diluted at a concentration of 4 ug/mL in free calcium/magnesium PBS buffer and incubated overnight at 4° C. After incubation, the coating solution was removed and the plates were blocked with 100 uL/well of PBS supplemented with 0.1% Tween-20+1% BSA for 1 h at 37° C.

Sixty microliters (60 uL) of two serial dilutions of individual mouse serum (starting dilution 1/10 in blocking buffer) was prepared into a 96-well clear V-bottom polypropylene microplate and mixed with 60 uL/well of biotinylated-hIgG antibodies pre-diluted at 0.7 ug/mL in blocking buffer. The blocking buffer from HSV-2 gE/gI coated plates was removed and 100 uL of the mixture was then transferred in the corresponding wells for an incubation period of 24h at 37° C. Positive control serum of the assay was a pool of serum samples. Negative control serum of the assay was a pool of irrelevant HPV serum samples diluted 1/1000 and mixed with hIgG.

After incubation, the plates were washed four times with PBS 0.1% Tween20 (washing buffer) and 50 uL/well of streptavidin-horseradish peroxidase diluted 2000× was added and plates were incubated for 30 min at 37° C. After incubation, plates were washed four times with washing buffer and 50 uL/well of a solution containing 75% single-component TMB peroxidase ELISA substrate diluted in sodium citrate 0.1M pH5.5 buffer were added for 10 min at room temperature. Enzymatic color development was stopped with 50 uL/well of 0.4N sulfuric acid 1M (H₂SO₄) and the plates were read at an absorbance of 450/620 nm using an ELISA reader. Optical densities (OD) were captured and fitted to a curve. Titers were expressed as the effective dilution at which 50% (i.e. ED50) of the signal was achieved by sample dilution.

For each plate and using a reference sample (i.e. irrelevant serum), the reference ED50 value was estimated using the following formula:

$$ED 50 = {OD}_{0 \%} + 0.5 * ( {OD}_{100 \%} - {OD}_{0 \%} )$$

where OD_{100%} is the highest OD obtained with similar samples and OD_{0%} is the lowest achievable signal. For each plate, the former was obtained by averaging (mean) 6 replicates while the latter was set at zero. Samples' ED50 titers were computed by way of linear interpolation between the left and right measurements closest to the ED50 estimate within the plate. The approximation was obtained, on the untransformed OD and the logarithm base 10 transformed dilutions.

Sample were not assigned a titer in the following cases:
no measurement was available above or below the ED50,
curve crossed at least twice the ED50 and
one of the dilution steps (left or right) closest to the ED50 was missing
   Evaluation of the Ability of Polyclonal Sera to Bind & Activate mFcγRIII after Incubation of HSV-2 gE/gI Transfected Cells (Mouse FcγRIII ADCC Bioassay):

The mouse FcγRIII Antibody Dependent Cell Cytotoxicity (ADCC) Reporter Bioassay, developed by Promega laboratory, is a bioluminescent cell-based assay which can be used to measure the ability of antibodies to specifically bind and activate the mouse FcγRIII expressed by modified Jurkat reporter cells after incubation with HSV-2 gE/gI transfected cells. Briefly, 3T3 cells, initially purchased from ATCC laboratories (clone A31, ATCC ref CCL-163), were grown in DMEM+10% heat inactivated FBS+1% L-glutamine 2 mM+1% Penicillin/streptomycin media. 3T3 cells (500 uL of cells at 1×10⁶ cells/mL) were transfected by electroporation with 20ug of HSV-2 gE/gI plasmid DNA in a 4 mm cuvette. After electroporation, all cuvettes were pooled to homogenise cell suspension and 500 μL of cell suspension/well was transferred into 6-well plates in 2 mL of pre-warmed DMEM+1% L-Glutamine 2 mM+1% Penicillin/streptomycin+10% of Ultra low IgG FBS media (electroporation media) and incubated at 37° C., 5% CO₂ during 48h. After 24h incubation, 2 mL of electroporation media was added in each well.

After 48h incubation, HSV-2 gE/gI transfected 3T3 cells (target cells (T)) were collected and pooled from the different 6-well plates. Cell suspension was centrifuged (10 min, 340 g, at RT) and resuspended in Promega assay buffer (96% RPMI+4% of low IgG serum) for cell counting. Then a solution at 96.000 3T3 cells/mL was prepared in Promega assay buffer and 25 μL of this suspension (24.000 cells/25 μL/well) was added in 96-well plates. In a round-bottom 96-well plates (Nunc, ref 168136), a 3-fold serial dilution of each mouse serum sample (starting dilution 1/500) in 200 μL was performed in Promega assay buffer and 25 μL of each dilution was transferred to the corresponding well containing already the HSV-2 gE/gI-transfected 3T3 cells. Finally, 25 μL of genetically engineered Jurkat cells expressing mouse FcγRIII (Effector Cells (E)) at a concentration of 2.400.000 cells/mL (60.000 cells/25 μL/well) were added in each well (˜E/T 2,5/1) and plates were incubated for 6h at 37° C. - 5% CO₂. After incubation, plates were put at RT for 15 min and 75 μL of Bio-Glow reagent were added in each well. The plates were finally incubated for 20 min at RT, read using luminometer and expressed in Relative Luminescence Units (RLU).

Samples titer in the mouse ADCC-like assay were computed by means of the area under the curve (AUC) as described in Huang, with minor modifications. Briefly, log-transformed responses (i.e. RLU) were fitted, on log 3-transformed sample serial dilutions, with a 5-parameter logistic model for each sample on the plate. A negative control sample (i.e. irrelevant mouse serum) was used to evaluate, in each plate, the background signal threshold estimated with its geometric mean plus 3 standard deviations (i.e. exp[mean(log RLU)+3*sd(log RLU)]). The area below the fitted curve of each sample and above the plate background (i.e. AUC) was computed using the trapezoid approximation method with 100 subdivisions used in the integration.

For sample curves sitting above the background (i.e. lower asymptote above plate background), the AUC was computed with the same method with a slight adaptation. First the AUC was obtained by integrating the 5PL model from the first to last serial dilution. Then the area under the plate background (i.e. also computed from the first to last serial dilution) was subtracted from it.

### Results

### Evaluation of Vaccine-Specific Antibody Responses

The anti-HSV-2 gE & gI-specific IgG antibody responses were investigated by ELISA at day 24 (24P1) and day 49 (21PII), and expressed in ELISA titers.

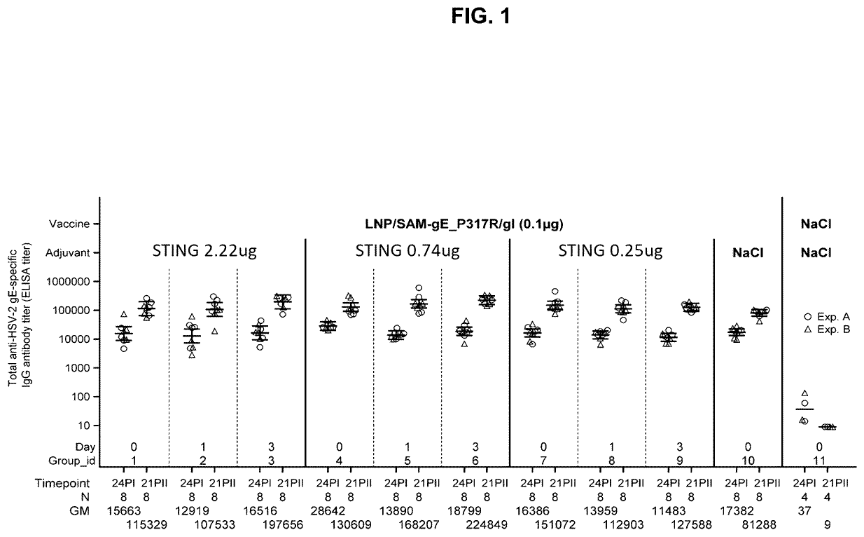

Anti-HSV-2 gE-specific IgG antibodies Adjuvanted 0.lug LNP/SAM-gE_P317/gI resulted in increased GM titers of anti-HSV-2 gE-specific IgG antibodies in 9 of 9 groups at 21PII, relative to unadjuvanted control (FIG. 1).

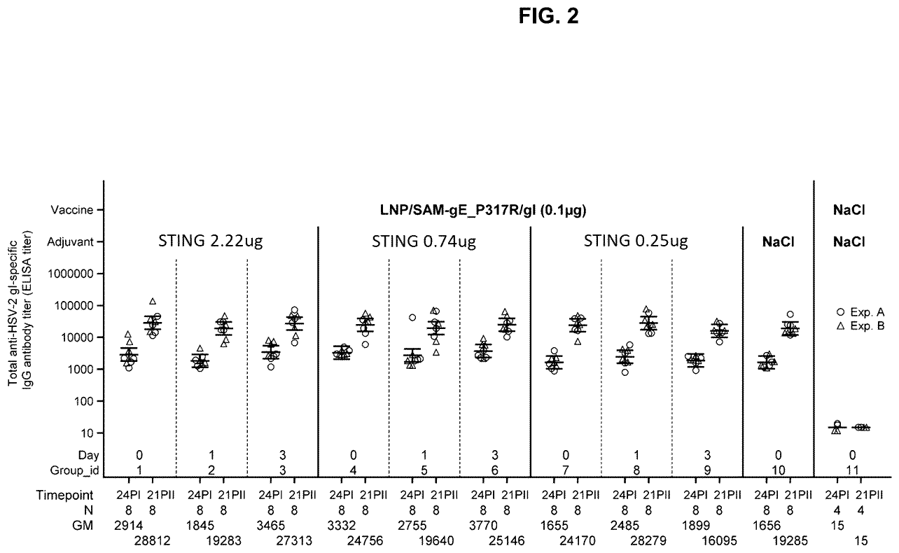

Anti-HSV-2 gI-specific IgG antibodies Adjuvanted 0.lug LNP/SAM-gE_P317/gI resulted in increased GM titers of anti-HSV-2 gI-specific IgG antibodies in 7 of 9 groups at 21PII, relative to unadjuvanted control FIG. 2).

Evaluation of Cross-Reactive Antibody Response to gE/gI from HSV-1

The anti-HSV-1 gE/gI cross-reactive IgG antibody response was investigated by ELISA at day 49 (21PII) and expressed in ELISA titers.

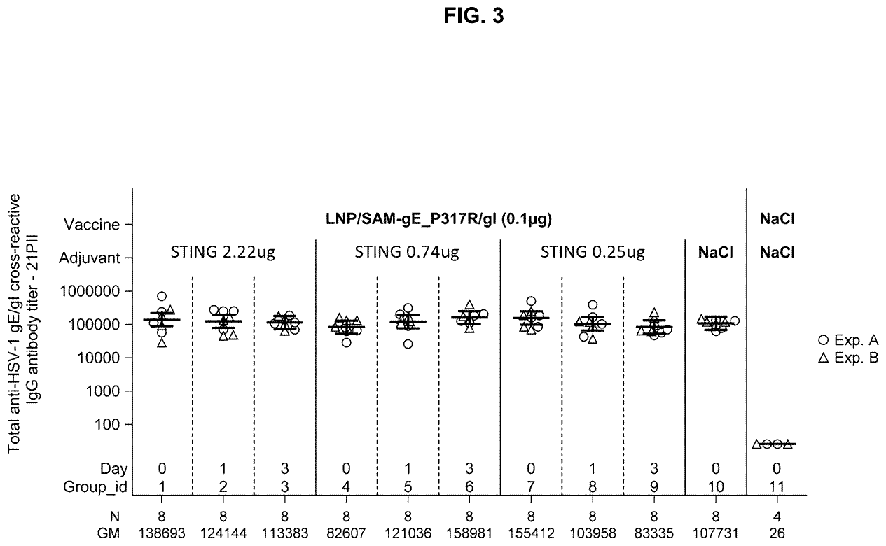

0.1 ug LNP/SAM-gE_P317/gI adjuvanted with 2.22ug of STING resulted in increased GM titers of anti-HSV-1 gE/gI cross reactive IgG antibody in all groups, relative to unadjuvanted control (FIG. 3).

### Evaluation of Vaccine-Specific Antibody Function 21 Days Post-Second Vaccination

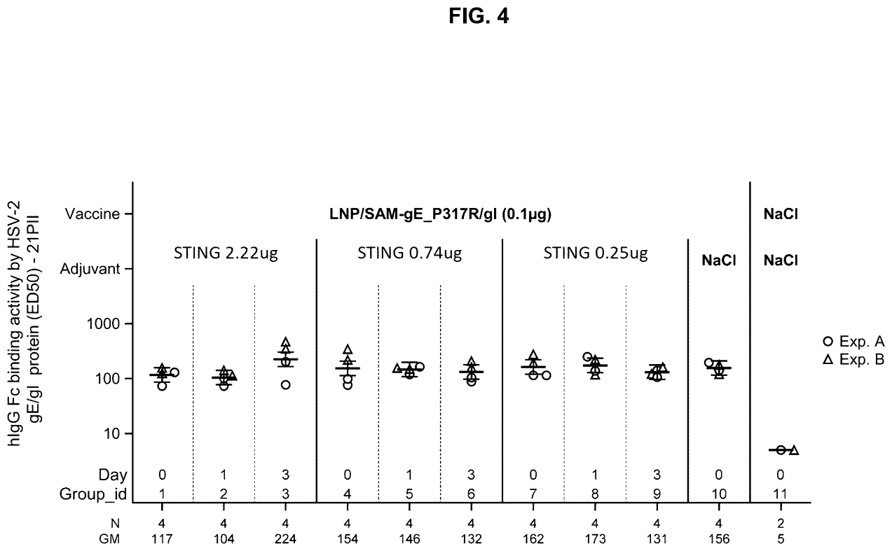

Vaccine-specific antibody functions were investigated in the sera collected at 21 days post-second immunization by assaying the ability of polyclonal antibodies to decrease binding of human IgG (hIgG) Fc by HSV-2 gE/gI protein. 4 pools of 2 mouse serum samples were prepared in each group, except for NaCl group where 2 pools of 2 mouse serum samples were prepared. The data are illustrated in FIG. 4.

### Evaluation of Vaccine-Specific T Cell Responses 21 Days Post-Second Vaccination

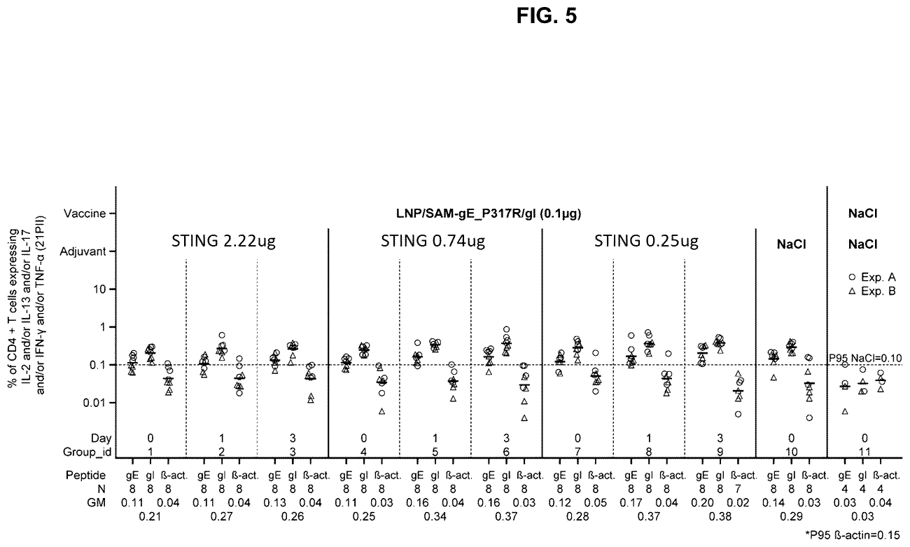

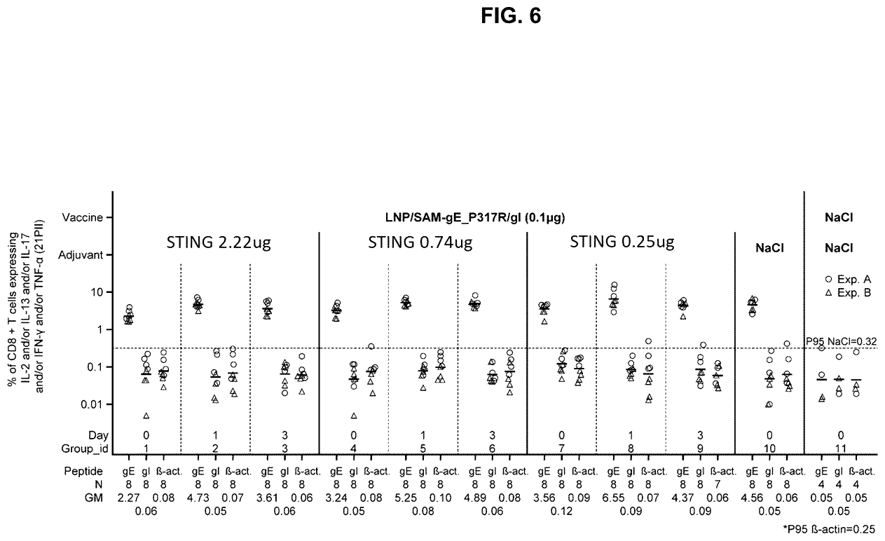

Twenty-one days after the second immunization (day 49), the anti-HSV-2 gE & gI-specific CD4+/CD8+ T cell responses were measured in spleens from mice. The data are illustrated in FIGS. 5 and 6.

### Example 2—Immunogenicity Evaluation of Non-Replicating mRNA Administered with a STING Agonist in Mice

### Materials and Methods

### Mouse Immunisation, Vector Production and Vaccination Scheme

Female CB6F1 inbred mice aged 6-8 weeks old were randomly assigned to study groups (n=8 for groups 1-12 (gr1-12), n=16 for groups 12-14 (gr13-14) and n=4 for group 15 (gr15)) and kept under pathogen-free conditions. Mice (gr-14) were intramuscularly (i.m.) immunized, in the gastrocnemius muscle, at days 0 & 21 with 0.8ug of LNP-formulated mRNA ((SEQ ID No:11, non-modified; SEQ ID No:12, uridine replacement by N1-methyl-pseudouridine—‘N1mψ’)) encoding HSV-2 gE (SEQ ID No:13) (25 uL volume) and were injected, in the same muscle, with different doses of STING agonist (0.74 or 2.22ug) prepared as detailed above under Example 1 (25 uL) either at the same time (day+0) or 1 or 3 days after each of the two mRNA immunizations. As positive controls for vaccine response, two groups of mice (gr(3-14) were injected i.m. with a saline solution (NaCl 150 mM, 25 uL) at the same time as mRNA. Finally, as a negative control, mice (gr15) were i.m injected with only 50 uL of a saline solution (NaCl 150 mM), following the same schedule of immunization (see Table 3 for details of each group).

TABLE 3
Summary of the study design and formulations tested

| | | | Vaccine | Adjuvant | |
|---|---|---|---|---|---|
| | | | immuni- | dose and | Volume |
| | | | zation | immuni- | and route |
| | Number | Vaccine | schedule | zation | of |
| Group | animals | name & dose | (Days) | schedule | injection |
| 1 | 8 | LNP/HSV-2 | Days | 2.22 μg | i.m. |
| | | gE mRNA | 0 & 21 | Day + 0 | 25 uL each |
| | | (native) | | | |
| 2 | 8 | LNP/HSV-2 | Days | 2.22 μg | i.m. |
| | | gE mRNA | 0 & 21 | Day + 1 | 25 uL each |
| | | (native) | | | |
| 3 | 8 | LNP/HSV-2 | Days | 2.22 μg | i.m. |
| | | gE mRNA | 0 & 21 | Day + 3 | 25 uL each |
| | | (native) | | | |
| 4 | 8 | LNP/HSV-2 | Days | 0.74 μg | i.m. |
| | | gE mRNA | 0 & 21 | Day + 0 | 25 uL each |
| | | (native) | | | |
| 5 | 8 | LNP/HSV-2 | Days | 0.74 μg | i.m. |
| | | gE mRNA | 0 & 21 | Day + 1 | 25 uL each |
| | | (native) | | | |
| 6 | 8 | LNP/HSV-2 | Days | 0.74 μg | i.m. |
| | | gE mRNA | 0 & 21 | Day + 3 | 25 uL each |
| | | (native) | | | |
| 7 | 8 | LNP/HSV-2 | Days | 2.22 μg | i.m. |
| | | gE mRNA | 0 & 21 | Day + 0 | 25 uL each |
| | | (N1mΨ) | | | |
| 8 | 8 | LNP/HSV-2 | Days | 2.22 μg | i.m. |
| | | gE mRNA | 0 & 21 | Day + 1 | 25 uL each |
| | | (N1mΨ) | | | |
| 9 | 8 | LNP/HSV-2 | Days | 2.22 μg | .m. |
| | | gE mRNA | 0 & 21 | Day + 3 | 25 uL each |
| | | (N1mΨ) | | | |
| 10 | 8 | LNP/HSV-2 | Days | 0.74 μg | i.m. |
| | | gE mRNA | 0 & 21 | Day + 0 | 25 uL each |
| | | (N1mΨ) | | | |
| 11 | 8 | LNP/HSV-2 | Days | 0.74 μg | i.m. |
| | | gE mRNA | 0 & 21 | Day + 1 | 25 uL each |
| | | (N1mΨ) | | | |
| 12 | 8 | LNP/HSV-2 | Days | 0.74 μg | i.m. |
| | | gE mRNA | 0 & 21 | Day + 3 | 25 uL each |
| | | (N1mΨ) | | | |
| 13 | 16 | LNP/HSV-2 | Days | (NaCl 150 mM) | i.m. |
| | | gE mRNA | 0 & 21 | Day + 0 | 25 uL each |
| | | (native) | | | |
| 14 | 16 | LNP/HSV-2 | Days | (NaCl 150 mM) | i.m. |
| | | gE mRNA | 0 & 21 | Day + 0 | 25 uL each |
| | | (N1mΨ) | | | |
| 15 | 4 | NaCl 150 mM | Days | (NaCl 150 mM) | i.m. |
| | | | 0 & 21 | Day + 0 | 25 uL each |

Serum samples were collected at days 21 & 35 post-prime immunization (21PI, 14P11) to measure anti-HSV-2 gE-specific and HSV-1 gE/gI cross-reactive IgG antibody responses and investigate the function of vaccine-specific antibodies. Spleens were also collected 35 days post-prime immunization (14P11) to evaluate anti-HSV-2 gE-specific and anti-HSV-1 gE/gI cross-reactive CD4+/CD8+ T cell responses. The study was split across two independent experiments—Experiment A (LIMS20210169) and Experiment B (LIMS20210170).

HSV gE non-replicating mRNA pDNA template was digested (linearized) with BspQ1 restriction enzyme, extracted with phenol: chloroform: isoamyl alcohol, washed with 70% ethanol, and resuspended in nuclease free water. In vitro transcription (IVT) reaction was performed with the linear template using a T7 Transcription kit at 37° C. for 4 hrs. IVT reactions contained either normal uridine or N1-Methyl-Pseudouridine-5′-Triphosphate. The IVT reactions were treated with TURBO DNase provided in the IVT kit, 15 minutes at 37° C., and the IVT product was lithium chloride purified and resuspended in nuclease free water. The IVT RNA was then capped using a m7G capping system with the addition of 2′-O-methyltransferase to achieve Cap-1 structure. The Capped IVT product was purified via silica column, and eluted in nuclease free water. The capped product was then treated with Anarctic phosphatase at 37° C. for 1 hr as per the manufacturer's recommendations. Finally, the Cap-1 IVT product was silica column purified as above and eluted in nuclease free water. The quality of the RNA was assessed by gel electrophoresis.

mRNA-LNP was prepared by rapidly mixing ethanolic solutions of lipids with aqueous buffers that contain the mRNA. The mRNA/lipid complexes are mixed to form lipid nanoparticles that entrap the RNA. Following this mixing step, the lipid nanoparticles matured to entrap the RNA and then were transferred to a final Tris/sucrose storage buffer through a buffer exchange step. The LNP solutions were then characterized for size, lipid content, RNA entrapment, final mRNA concentration, and in vitro potency. The LNP solutions comprised 20 mM Tris, 5 mM NaCl, 7.5% sucrose, and had a pH of 8.

### Detection of Total Anti-HSV-2 gE IqG Antibodies by ELISA

Quantification of the total anti-HSV-2 gE-specific IgG antibodies was performed using indirect ELISA. Recombinant HSV-2 gE (˜51 kDa) was used as coating antigen.

Polystyrene 96-well ELISA plates were coated with 100 uL/well of antigen diluted at a concentration of 2 ug/mL (HSV-2 gE) and 1 ug/mL (HSV-2 gI) in carbonate/bicarbonate 50 mM pH 9.5 buffer and incubated overnight at 4° C. After incubation, the coating solution was removed and the plates were blocked with 200 uL/well of skimmed milk media additive 10% diluted in PBS (blocking buffer) for 1 h at 37° C. The blocking solution was removed and three-fold sera dilutions (in PBS+0.1% Tween20+1% BSA buffer) were added to the coated plates and incubated for 1h at 37° C. The plates were washed four times with PBS 0.1% Tween20 (washing buffer) and peroxidase conjugated goat anti-mouse IgG (H+L) was used as secondary antibody. One hundred microliters per well of the secondary antibody diluted at a concentration of 1:500 in PBS+0.1% Tween20+1% BSA buffer was added to each well and the plates were incubated for 45 min at 37° C.

The plates were then washed four times with washing buffer and 2 times with deionised water and incubated for 10 min at RT (room temperature) with 100 uL/well of a solution of 75% single-component TMB peroxidase ELISA substrate diluted in sodium citrate 0.1M pH5.5 buffer. Enzymatic color development was stopped with 100 uL of 0.4N sulfuric acid 1M (H₂SO₄) per well and the plates were read at an absorbance of 450/620 nm using an ELISA reader.

Optical densities (OD) were captured and analysed. A standard curve was generated by applying a 4-parameter logistic regression fit to a reference standard. Antibody titer in the samples was calculated by interpolation of the standard curve. The antibody titer of the samples was obtained by averaging the values from dilutions that fell within the 20-80% dynamic range of the standard curve. ELISA titers were normalized using a sample reference to allow titer comparisons.

Detection of Total Anti-HSV-1 gE/gI Cross-Reactive IqG Antibodies Measured by ELISA

Quantification of the total anti-HSV-1 gE/gI-specific IgG antibodies was performed using indirect ELISA. Recombinant gE/gI heterodimer protein from HSV-1 was used as coating antigen. Polystyrene 96-well ELISA plates were coated with 100 μL/well of recombinant HSV-1 gE/gI protein diluted at a concentration of 2 pg/mL in carbonate/bicarbonate 50 mM pH 9.5 buffer and incubated overnight at 4° C. After incubation, the coating solution was removed and the plates were blocked with 200 μL/well of skimmed milk media additive 10% diluted in PBS (blocking buffer) for 1 h at 37° C. The blocking solution was removed and a three fold serial dilution of each sera was prepared (in PBS+0.1% Tween20+1% BSA buffer) and added to the coated plates for 1h at 37° C. After incubation, the plates were washed four times with PBS 0.1% Tween20 (washing buffer) and 100 μl/well of peroxidase conjugated goat anti-mouse IgG (H+L) diluted at a concentration of 1:500 in PBS+0.1% Tween20+1% BSA buffer was added to each well for 45 min at 37° C. The plates were then washed four times with washing buffer and 2 times with deionised water and incubated for 10 min at RT (room temperature) with 100 μL/well of 75% single component TMB peroxidase ELISA substrate diluted in sodium citrate 0.1M pH5.5 buffer. Enzymatic colour development was stopped with 100 μL of 0.4N sulfuric acid 1M (H₂SO₄) per well and the plates were read at an absorbance of 450/620 nm using an ELISA reader. Optical densities (OD) were captured and analysed. A standard curve was generated by applying a 4-parameter logistic regression fit to a reference standard. Antibody titer in the samples was calculated by interpolation of the standard curve. The antibody titer of the samples was obtained by averaging the values from dilutions that fell within the 20-80% dynamic range of the standard curve. ELISA titers were normalized using a standard reference to allow titer comparisons.

Evaluation of Anti-HSV-2 gE-Specific and Anti-HSV-1 gE Cross-Reactive CD4+/CD8+ T Cell Responses by Intracellular Cytokine Staining (ICS)

The frequencies of HSV-2 gE-specific and HSV-1 gE cross-reactive CD4+ and CD8+ T-cells producing IL-2 and/or IFN-γ and/or TNF-α (Th1) and/or IL-17 (Th17) were evaluated in splenocytes collected 14 days second immunization after ex-vivo stimulation with HSV-2 gE or HSV-1 gE peptides pools.

### Isolation of Splenocytes

Spleens were collected from individual mice 14 days after second immunization and placed in RPMI 1640 medium supplemented with RPMI additives (glutamine, penicillin/streptomycin, sodium pyruvate, non-essential amino-acids & 2-mercaptoethanol) (=RPMI/additives). Cell suspensions were prepared from each spleen using a tissue grinder. The splenic cell suspensions were filtered (cell stainer 100 um) and then the filter was rinsed with 35 mL of cold PBS-EDTA 2 mM. After centrifugation (335 g, 10 min at 4° C.), cells were resuspended in 5 mL of cold PBS-EDTA. A second washing step was performed, as previously described, and the cells were finally resuspended in 2 mL of RPMI/additives supplemented with 5% FCS. Cell suspensions were then diluted 20× (10 uL) in PBS buffer (190 uL) for cell counting. After counting, cells were centrifuged (335 g, 10 min at RT) and resuspended at 10⁷ cells/mL in RPMI/additives supplemented with 5% FCS.

### Cell Preparation

Fresh splenocytes were seeded in round bottom 96-well plates at 10⁶ cells/well (100 uL). The cells were then stimulated for 6 hours (37° C., 5% CO₂) with anti-CD28 and anti-CD49d antibodies at 1ug/mL per well, containing 100 uL of either:
15 mer overlapping peptide pool covering the sequences of gE protein from HSV-2 (1ug/mL per peptide per well).
15 mer overlapping peptide pool covering the sequences of gE protein from HSV-1 (1ug/mL per peptide per well).
15 mer overlapping peptide pool covering the sequences of human p-actin protein (1ug/mL per peptide per well) (irrelevant stimulation).
RPMI/additives medium (as negative control of the assay).
PMA—ionomycin solution at working concentrations of 0.25 ug/mL and 2.5 ug/mL respectively (as positive control of the assay).

After 2 hours of ex vivo stimulation, brefeldin A protein transport inhibitor diluted 1/200 in RPMI/additives supplemented with 5% FCS was added for 4 additional hours to inhibit cytokine secretion. Plates were then transferred at 4° C. for overnight incubation.

### Intracellular Cytokine Staining

After overnight incubation at 4° C., cells were transferred to V-bottom 96-well plates, centrifuged (189 g, 5 min at 4° C.) and washed with 250 uL of cold PBS+1% FCS (flow buffer). After a second centrifugation (189 g, 5 min at 4° C.), cells were resuspended to block unspecific antibody binding (10 min at 4° C.) in 50 uL of flow buffer containing anti-CD16/32 antibodies diluted 1/50. Then, 50 uL flow buffer containing mouse anti-CD4-V700 antibodies, anti-CD8-PerCp-Cy5.5 antibodies and fixable yellow dead cell stain, anti-CD62L BV786, anti-CD127 BV421 was added for 30 min in darkness at 4° C. After incubation, 100 uL of flow buffer was added into each well and cells were then centrifuged (189 g for 5 min at 4° C.). A second washing step was performed with 200 uL of flow buffer and after centrifugation, cells were fixed and permeabilized by adding 200 uL of fixation solution for 20 min at 4° C. in darkness. After plate centrifugation (500 g for 5 min at 4° C.), cells were washed with 200 uL of cell wash buffer, centrifuged (500 g for 5 min 4° C.) and resuspended in 50 uL of cell wash buffer containing anti-IL2-FITC, anti-IFN-γ-APC and anti-TNF-α-PE, anti-IL-13 PE Cy7, anti-IL-17 BV605 antibodies, for 1 hour at 4° C. in darkness. After incubation, 100 uL of flow buffer was added into each well and cells were then finally washed with 200 uL of cell wash buffer (centrifugation 500 g for 5 min at 4° C.) and resuspended in 220 uL PBS.

### Cell Acquisition and Analysis

Stained cells were acquired by flow cytometry and analyzed. Live cells were identified with staining and then lymphocytes were isolated based on forward/side scatter lights (FSC/SSC) gating. The acquisition was performed on ˜20,000 CD4+/CD8+ T-cell events. The percentages of IFN-γ^{+/-}, IL-2^{+/-}, TNF-α^{+/-} and IL-17^{+/-} producing cells were calculated on CD4+ and CD8+ T cell populations (IL-13 was not analysed). For each sample, unspecific signal detected after medium stimulation was removed from the specific signal detected after peptide pool stimulation.

Competitive ELISA to Evaluate the Ability of Vaccine-Specific Antibodies to Decrease Human IqG Fc Binding by HSV-2 gE/gI Protein

The ability of polyclonal sera collected in different groups of mice to decrease in-vitro hIgG Fc binding by recombinant HSV-2 gE/gI protein was investigated by competitive ELISA. Recombinant HSV-2 gE/gI protein was used as coating antigen.

Polystyrene 96-well ELISA plates were coated with 50 uL/well of HSV-2 gE/gI protein diluted at a concentration of 4ug/mL in free calcium/magnesium PBS buffer and incubated overnight at 4° C. After incubation, the coating solution was removed and the plates were blocked with 100 uL/well of PBS supplemented with 0.1% Tween20+1% BSA for 1 h at 37° C.

Sixty microliters (60 uL) of two serial dilution of individual mouse serum (starting dilution 1/10 in blocking buffer) was prepared into 96-well clear V-bottom polypropylene microplate and mixed with 60 uL/well of biotinylated-hIgG antibodies pre-diluted at 0.7ug/mL in blocking buffer. The blocking buffer from HSV-2 gE/gI coated plates was removed and 100 uL of the mixture was then transferred in the corresponding wells for an incubation period of 24h at 37° C. Positive control serum of the assay was a pool of serum samples from mice immunized with HSV-2 gE/gI antigen in previous experiments. Negative control serum for the assay was a pool of irrelevant HPV serum samples diluted 1/1000 and mixed with hIgG.

After incubation, the plates were washed four times with PBS 0.1% Tween20 (washing buffer) and 50 uL/well of streptavidin-horseradish peroxidase diluted 2000× was added on the well and plates were incubated for 30 min at 37° C. After incubation, plates were washed four times with washing buffer and 50 uL/well of a solution containing 75% single-component TMB peroxidase ELISA substrate diluted in sodium citrate 0.1M pH5.5 buffer were added for 10 min at room temperature. Enzymatic color development was stopped with 50 uL/well of 0.4N sulfuric acid 1M (H₂SO₄) and the plates were read at an absorbance of 450/620 nm using an ELISA reader. Optical densities (OD) were captured and fitted to a curve. Titers were expressed as the effective dilution at which 50% (i.e. ED50) of the signal was achieved by sample dilution. For each plate and using a reference sample (i.e. irrelevant serum), the reference ED50 value was estimated using the following formula:

$$ED 50 = {OD}_{0 \%} + 0.5 * ( {OD}_{100 \%} - {OD}_{0 \%} )$$

where OD_{100%} is the highest OD obtained with similar samples and OD_{0%} is the lowest achievable signal. For each plate, the former was obtained by averaging (mean) 6 replicates while the latter was set at zero. The samples' ED50 titers were computed by way of linear interpolation between the left and right measurements closest to the ED50 estimate within the plate. The approximation was obtained, on the untransformed OD and the logarithm base 10 transformed dilutions.

Samples were not assigned a titer in the following cases:
no measurement was available above or below the ED50,
curve crossed at least twice the ED50 and
one of the dilution step (left or right) closest to the ED50 was missing.

### Results

### Evaluation of Vaccine-Specific Antibody Responses

The anti-HSV-2 gE-specific IgG antibody response was investigated by ELISA at day 21 (21PI) and day 35 (14PII) while the anti-HSV-1 gE/gI cross-reactive IgG antibody response was only investigated at day 35 (14PII). Results are expressed in ELISA titers and normalized using a sample reference to allow titers comparisons between groups.

Anti-HSV-2 gE-Specific IgG Antibodies

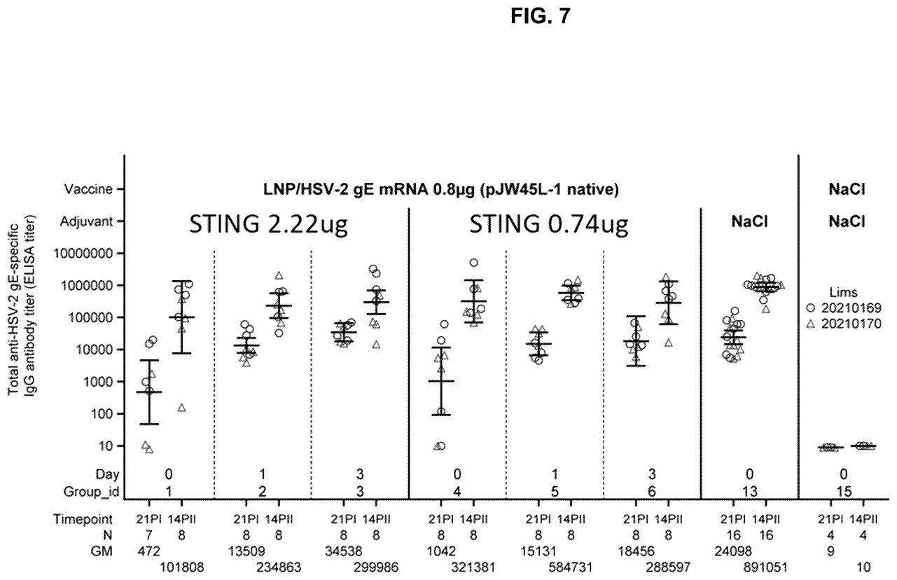

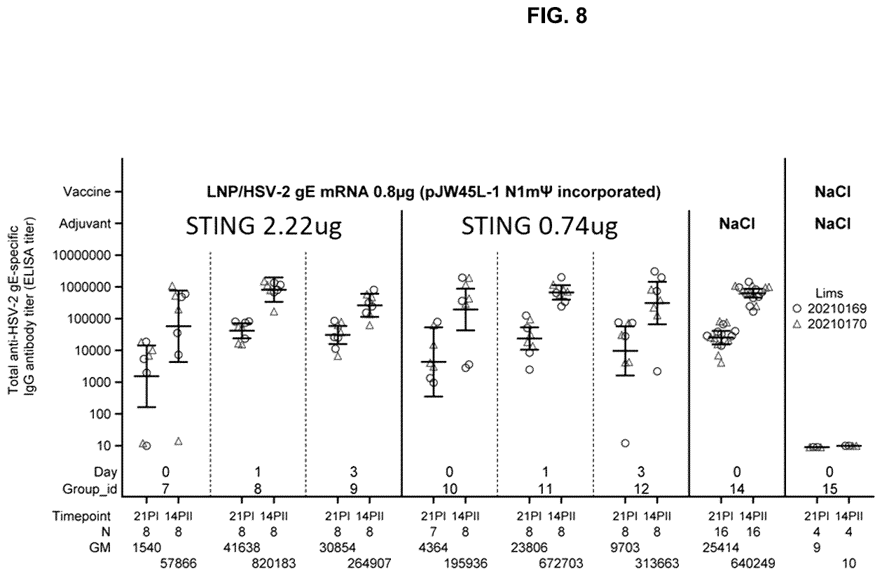

The results for anti-HSV-2 gE-specific IgG antibodies are shown in FIG. 7 (native mRNA) and FIG. 8 (N1m4).

Evaluation of Total Anti-HSV-1 gE/gI Cross-Reactive IqG Antibodies Measured by ELISA

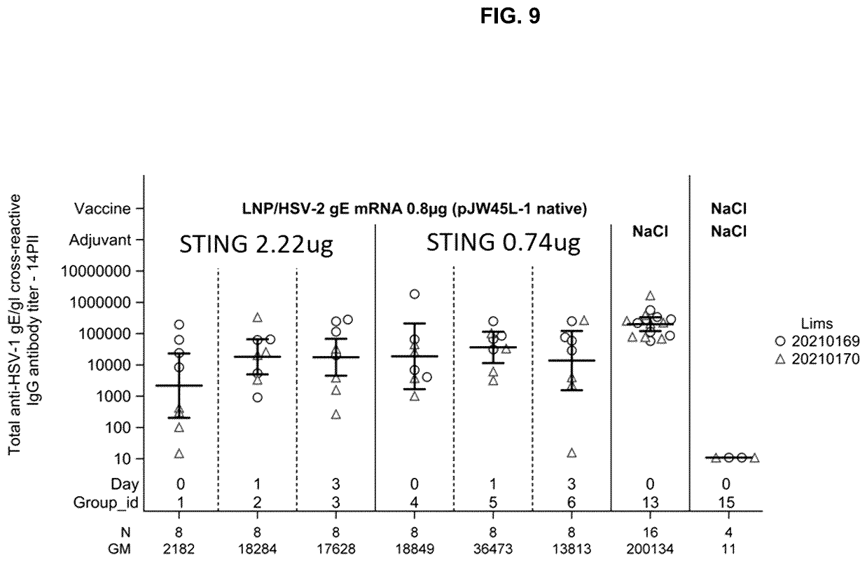

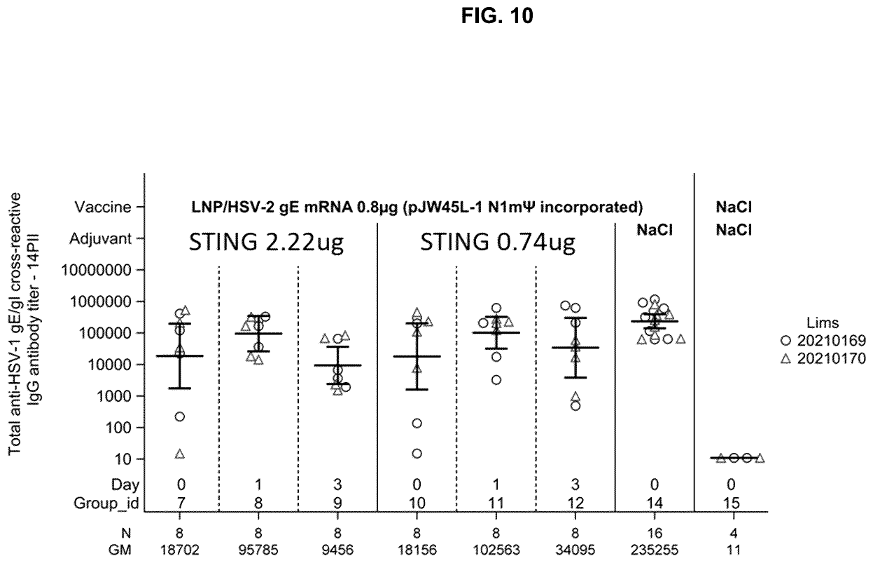

The results for anti-HSV-1 gE/gI cross-reactive IgG antibodies are shown in FIG. 9 (native mRNA) and FIG. 10 (N1m4).

### Evaluation of Vaccine-Specific Antibody Function 14 Days Post-Second Vaccination

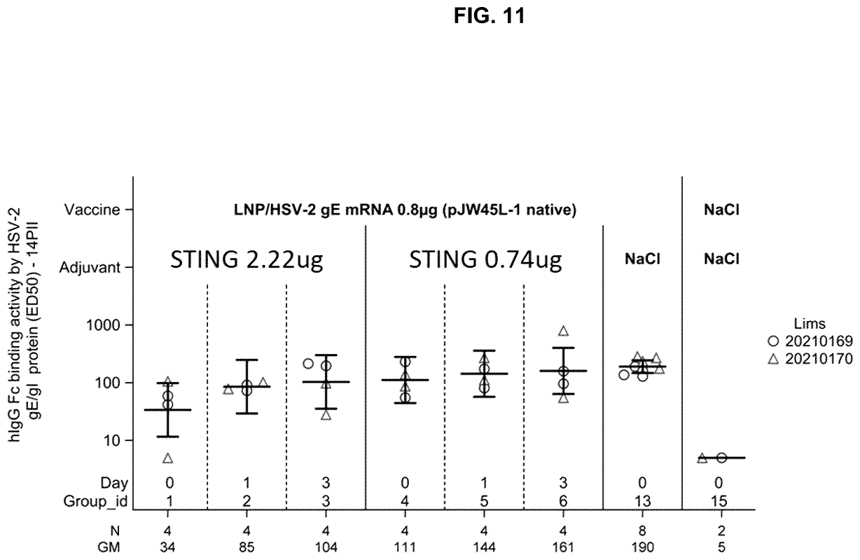

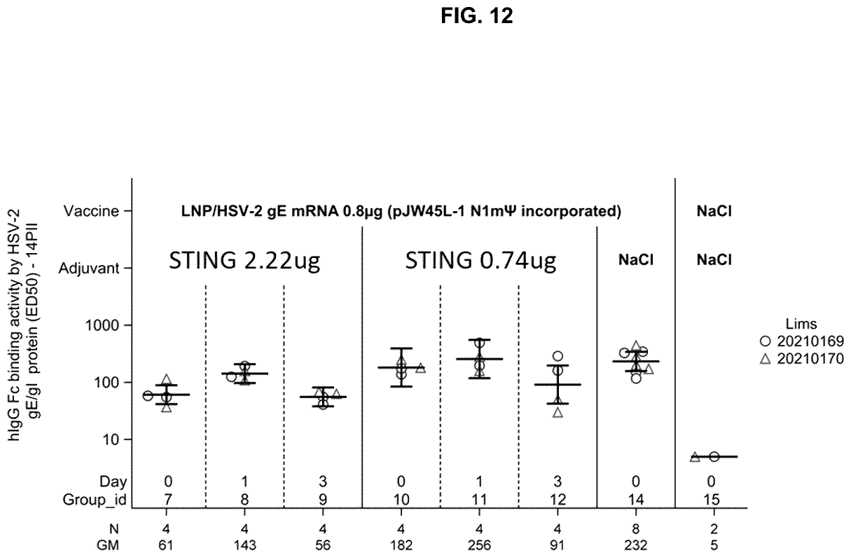

Vaccine-specific antibody functions were investigated in the sera collected at 14 days post-second immunization by assaying the ability of polyclonal antibodies to decrease binding of human IgG (hIgG) Fc by HSV-2 gE/gI protein. Results are shown in FIG. 11 (native mRNA) and FIG. 12 (N1m4).

### Evaluation of Vaccine-Specific T Cell Responses 14 Days Post-Second Vaccination

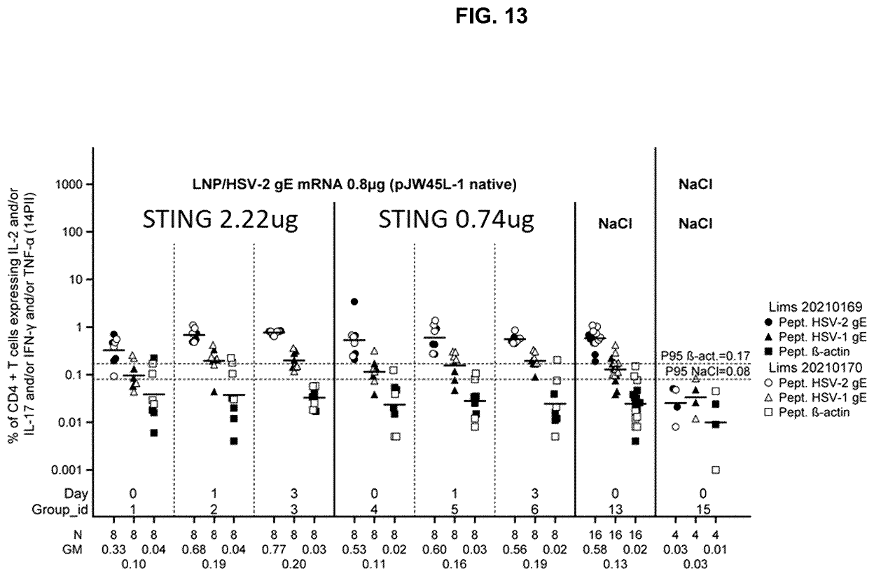

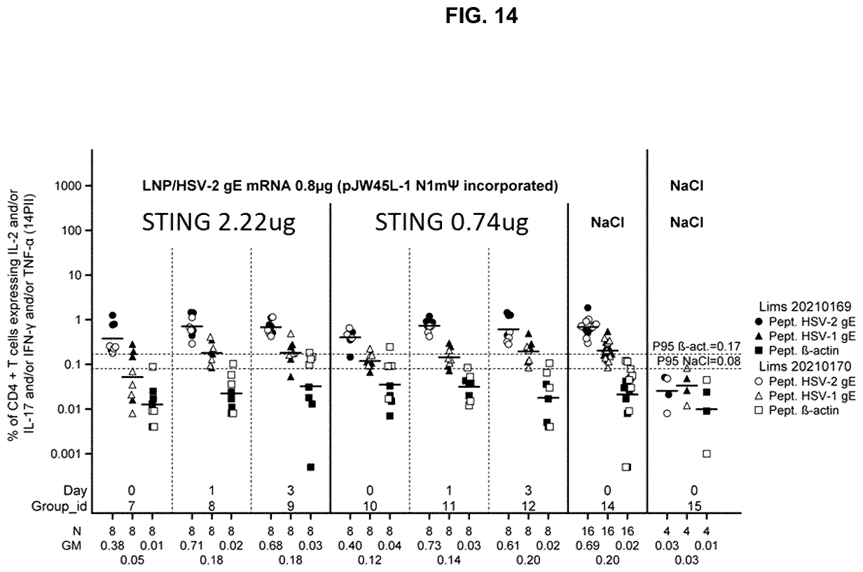

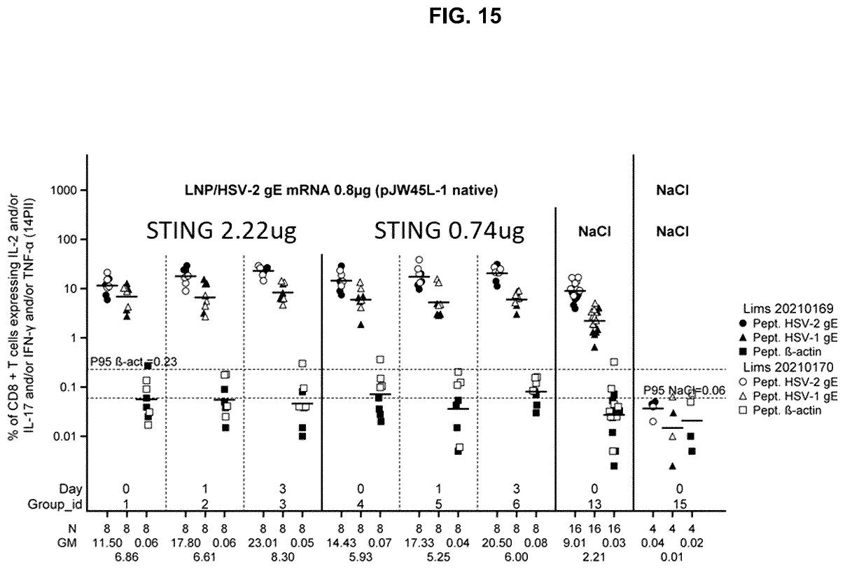

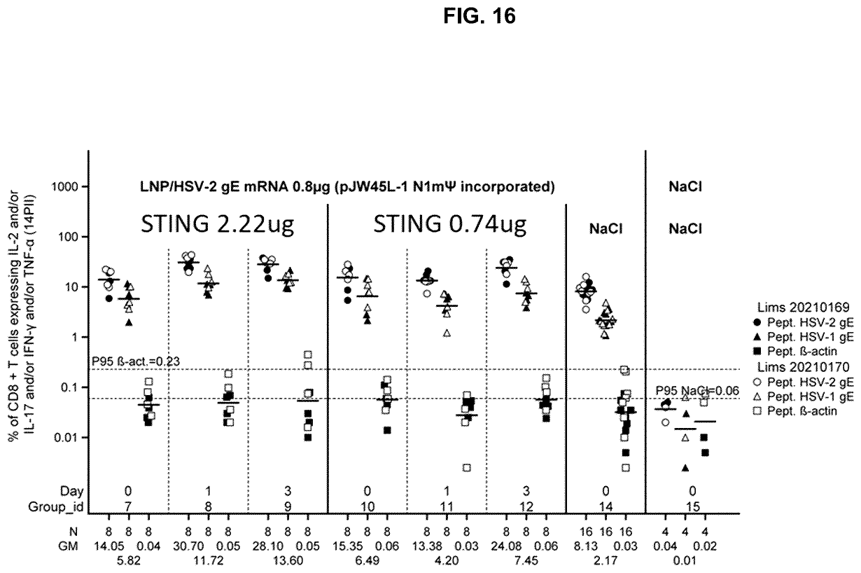

Fourteen days after the second immunization (day 35), the anti-HSV-2 gE-specific and anti-HSV-1 gE cross-reactive CD4+/CD8+ T cell responses were measured in the spleen. Results are provided in FIGS. 13 to 16.

### REFERENCES

Brito, L. A et al. A cationic nanoemulsion for the delivery of next-generation RNA vaccines, Molecular Therapy, 2014, 22,12, 2118-2129.
Bosch et al., The Coronavirus Spike Protein Is a Class I Virus Fusion Protein: Structural and Functional Characterization of the Fusion Core Complex J. Virol. 77:8801-8811 (2003)
Dayhoff M, O et al. A model of evolutionary change in proteins, Atlas of Protein Sequence and Structure, 1978 vol 5, supp. 3.
Dubensky TW Jr, et al. Rationale, progress and development of vaccines utilizing STING-activating cyclic dinucleotide adjuvants, Ther Adv Vaccines 2013 1(4):131-43
Jiang S, et al., Lipidoid-Coated Iron Oxide Nanoparticles for Efficient DNA and siRNA delivery Nano Lett., 13:1059-1064 (2013).
Kirchdoerfer et al., Stabilized coronavirus spikes are resistant to conformational changes induced by receptor recognition or proteolysis Sci. Rep. 8:15701 (2018)
Klucker M-F, et al. AF03, an Alternative Squalene Emulsion-Based Vaccine Adjuvant Prepared by a Phase Inversion Temperature Method J Pharm Sci. 2012 101(12):4490-4500
Li, Structure, Function, and Evolution of Coronavirus Spike Proteins Annu. Rev. Virol. 3:237-261 (2016)
Liu et al., Improvement of Pharmacokinetic Profile of TRAIL via Trimer-Tag Enhances Its Antitumor Activity in Vivo Sci Rep. 7:8953 (2017)
Norbert Pardi, Michael J. Hogan, Frederick W. Porter and Drew Weissman Nature Reviews Drug Discovery 2018 17:261-279
Pallesen et al., Immunogenicity and structures of a rationally designed prefusion MERS-CoV spike antigen Proc. Natl. Acad. Sci. U.S.A. 114:E7348-E7357 (2017)
Shah R, et al. The impact of size on particulate vaccine adjuvants. Nanomedicine (Lond) 2014 9:2671-2681
Shah R, et al. The development of self-emulsifying oil-in-water emulsion adjuvant and an evaluation of the impact of droplet size on performance. J Pharm Sci. 2015 104:1352-1361
Shah R, et al. The droplet size of emulsion adjuvants has significant impact on their potency, due to differences in immune cell-recruitment and -activation Scientific Reports 2019 9:11520
Wrapp et al., Cryo-EM structure of the 2019-nCoV spike in the prefusion conformation Science 367:1260-1263 (2020)
Zhang, C et al. Advances in mRNA vaccines for infectious diseases, Front. Immunol. 2019, 10:594.
Handbook of Pharmaceutical Excipients (eds. Rowe, Sheskey, & Quinn) 6th edition, 2009 ISBN 978-0-85369-792-3
Light Scattering from Polymer Solutions and Nanoparticle Dispersions Schartl W. 2007 ISBN 978-3-540-71950-2
(https://www.who.int/emergencies/diseases/novel-coronavirus-2019; accessed 16 Jun. 2020).
WO2018/176103
WO2017/162265
WO2005/113782
WO2016/172396
WO2017/070620
WO2012/006376
WO2012/030901
WO2012/031046
WO2012/031043
WO2012/006378
WO2011/076807
WO2013/033563
WO2013/006825
WO2014/136086
WO2015/095340
WO2015/095346
WO2016/037053
WO2012/006380
WO2013/006837
WO2013/006834
WO2017/175147
U.S. Pat. No. 4,373,071
U.S. Pat. No. 4,458,066
U.S. Pat. No. 4,500,707
U.S. Pat. No. 4,668,777
U.S. Pat. No. 4,973,679
U.S. Pat. No. 5,047,524
U.S. Pat. No. 5,132,418
U.S. Pat. No. 5,153,319
U.S. Pat. No. 5,262,530
U.S. Pat. No. 5,700,642
US 2021/0101924

1. A method of eliciting an immune response against an antigen in a subject, the method comprising administering to the subject (i) a carrier-formulated mRNA encoding the antigen and (ii) an adjuvant comprising a stimulator of interferon genes (STING) agonist.

2-6. (canceled)

7. A kit comprising:
(i) a first container comprising carrier-formulated mRNA encoding an antigen; and
(ii) a second container comprising an adjuvant comprising a STING agonist.

8. (canceled)

9. An immunogenic composition comprising: (i) a carrier-formulated mRNA encoding an antigen and (ii) an adjuvant comprising a STING agonist.

10. The immunogenic composition of claim 9, wherein the STING agonist is a small molecule.

11.-16. (canceled)

17. The immunogenic composition of claim 9, wherein the STING agonist is a nucleic acid, a protein, or a peptide.

18. The immunogenic composition of claim 10, wherein the STING agonist is a modified or unmodified cyclic dinucleotide.

19. The immunogenic composition of claim 10, wherein:
the STING agonist is selected from: a compound of one of Formula (I), Formula (II), and Formula (III); a pharmaceutically acceptable salt thereof: a tautomer thereof; and any combination thereof;
Formula (I) is: 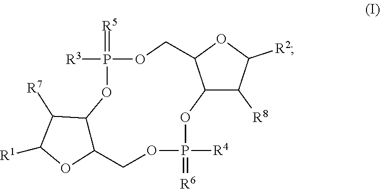
Formula (II) is: 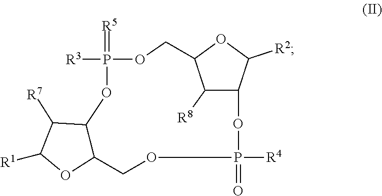
Formula (III) is: 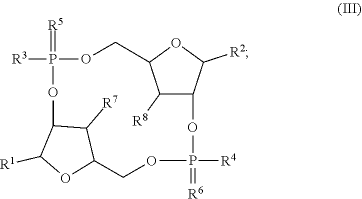
where in each of Formula (I), Formula (II), and Formula (III):
R¹ and R² are each independently selected from: 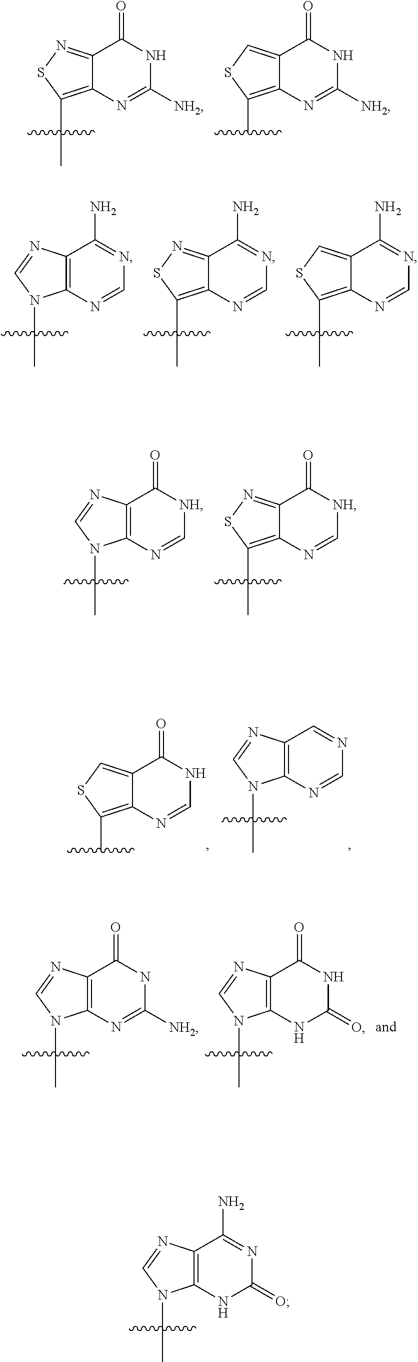
R³ and R⁴ are each independently —SH or —OH;
R⁵ and RP are oxygen or sulphur; and
R⁷ and R⁸ are each independently a halogen, hydrogen, —OH, or OCH₃.

20. The immunogenic composition of claim 10, wherein the STING agonist is 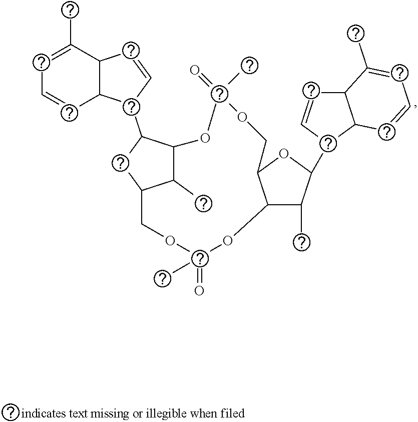
a pharmaceutically acceptable salt thereof, a tautomer thereof, or any combination thereof.

21. The immunogenic composition according to claim 10, wherein the STING agonist is: c-di^{tz}GMP, c-di-^{th}GMP, c-G^{tz}GMP, c-GAMP, c-^{th}GMP, c-^{tz}GMP, c-di-^{th}AMP c-di^{tz}AMP, c-di-AMP, c-di-GMP, c-diXGMP, c-G^{th}XMP, c-GXMP, c-ACMP, c-A^{th}XMP, c-A^{tz}XMP, c-di-^{th}XMP, or c-di^{tz}XMP.

22. The immunogenic composition of claim 10, wherein the STING agonist is:
2′,3′-cGAMP,
3′,3′-cGAMP, 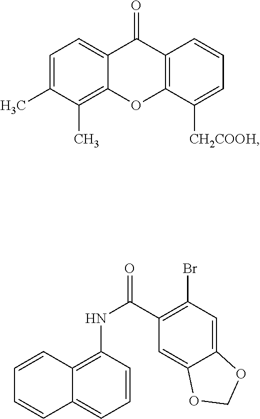
(6-bromo-N-(naphthalen-1-yl)benzo[d][1,3]dioxole-5-carboxamide),
or a pharmaceutically acceptable salt thereof.

23.-25. (canceled)

26. The immunogenic composition of claim 10, wherein:
the STING agonist is a compound of Formula A, a pharmaceutically acceptable salt thereof, a tautomer thereof, or any combination thereof;
Formula A is: 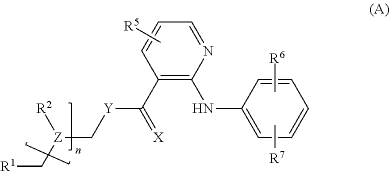
wherein:
X is O or NR^{4A};
Y is O, NR^{4A}, CH₂, or absent;
n is 0, 1, 2, or 3;
R¹ and R² are independently selected from OH, OR³, OR^{3A}, SR³, and NR³R⁴;
R³, R⁴, and R^{4A} are independently selected from hydrogen, a C₁-C₁₀ alkyl optionally substituted with 1-6 halogen, a C₆-C₁₀ aryl, and a 5-10 membered heteroaryl or R³ and R⁴ together with the nitrogen atom to which they are attached form a 3 to 7 membered heterocycle or a 5 to 10 membered heteroaryl;
R^{3A} is 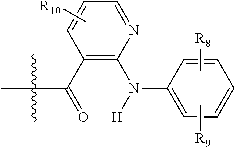
wherein [symbol] represents the point of connection of R^{3A} to the remainder of the molecule;
R⁵-R¹⁰ are independently selected from hydrogen, a halogen, a pseudohalogen, a C1-C10 alkyl optionally substituted with 1-6 halogens, a C6-C10 aryl, and a 5 to 10 membered heteroaryl.

27. The immunogenic composition of claim 10, wherein the STING agonist is: 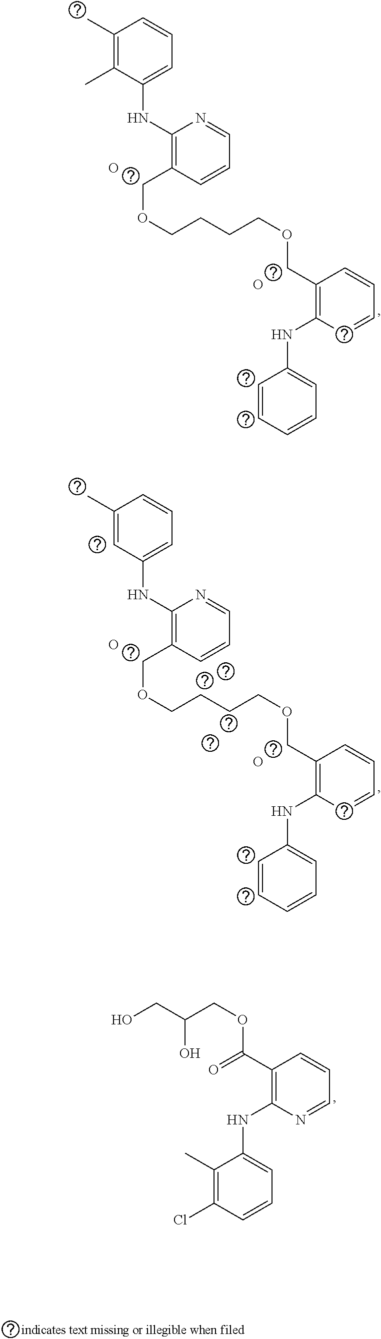
or a pharmaceutically acceptable salt thereof.

28. The immunogenic composition of claim 10, wherein the STING agonist is a flavonoid.

29. The immunogenic composition of claim 28, wherein the STING agonist comprises 10-(carboxymethyl)-9(10H)acridone, 5,6-Dimethylxanthenone-4-acetic acid, methoxyvone, 6,4′-dimethoxyflavone, 4′-methoxyflavone, 3′,6′-dihydroxyflavone, 7,2′-dihydroxyflavone, daidzein, formononetin, retusin 7-methyl ether, or xanthone.

30. The immunogenic composition according to claim 10T wherein:
the STING agonist is a compound of Formula B, a pharmaceutically acceptable salt thereof, a tautomer thereof, or any combination thereof; and
Formula B is: 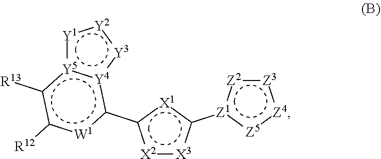
wherein: [symbol]
represents two conjugated double bonds in each of the five-membered rings and three conjugated double bonds in the six-membered ring,
W¹ is selected from CR¹¹ and N;
X¹ is selected from CR¹, C(R¹)₂, N, NR¹, O and S;
X² is selected from CR², C(R²)₂, N, NR², O, and S;
X³ is selected from CR³, C(R³)₂, N, NR³, O, and S;
where two or three of X¹, X², and X³ are independently selected from N, NR¹, NR², NR³, O, and S; and
where at least one of X¹, X², and X³ is selected from N, NR¹, NR², and NR³;
Y¹ is selected from N, NR⁴, O, S, CR⁴, and C(R⁴)₂;
Y² is selected from N, NR⁵, O, S, CR⁵, and C(R⁵)₂;
Y³ is selected from N, NR⁶, O, S, CR⁶, and C(R⁶)₂;
Y⁴ is selected from C and N;
Y⁵ is selected from C and N;
where at least one and not more than two of Y¹, Y², and Y³ are independently selected from N, NR⁴, NR⁵, and NR⁶;
where when Y⁴ is N, Y⁵ is C;
where when Y⁴ is C, Y⁵ is N;
Z¹ is selected from C and N;
Z² is selected from N, NR⁸, and CR⁸;
Z³ is selected from N, NR⁹, and CR⁹;
Z⁴ is selected from N, NR¹⁰, and CR¹⁰;
Z⁵ is selected from N, NR⁷, and CR⁷;
where two or three of Z¹, Z², Z³, Z⁴, and Z⁵ are independently selected from N, NR⁷, NR⁸, NR⁹, and NR¹⁰;
each R¹ is independently selected from H, a C₁-C₈ alkyl, a C₁-C₈ alkylene-NRR, and a C₁-C₈ alkylene-C(O)OR;
each R² is independently selected from H, a C₁-C₈ alkyl, a C₁-C₈ alkylene-NRR, a C₁-C₈ alkylene-C(O)OR, a C₁-C₈ alkylene-OR, and a C₁-C₈ alkylene-O—P(O)(OH)₂;
each R³ is independently selected from the group consisting H, a C₁-C₈ alkyl, a C₁-C₈ alkylene-NRR, a C₁-C₈ alkylene-C(O)OR, and a C₁-C₈ alkylene-O—P(O)(OH)₂;
each R⁴ is independently selected from the H, —OR, —NRR, a C₁-C₈ alkyl which is optionally substituted with one or two —OR, a C₁-C₈ alkylene-NRR, —C(O)OR, a C₁-C₈ alkylene-C(O)OR, a 3-10 membered heterocycle, a C₁-C₈ alkylene-3-10 membered heterocycle which is optionally substituted with one 3-10 membered heterocycle, a (C₃-C₁₀)-cycloalkyl, and a C₁-C₈ alkylene-(C₃-C₁₀)-cycloalkyl;
each R⁵ is independently selected from H, OR, a C₁-C₈ alkyl, —NRR, a C₁-C₈ alkylene-NRR, —C(O)OR, a C₁-C₈ alkylene-C(O)OR, a 3-10 membered heterocycle, a C₁-C₈ alkylene-3-10 membered heterocycle which is optionally substituted with one 3-10 membered heterocycle, and a C₁-C₈ alkylene-OR;
each R⁶ is H;
R⁷ is selected from H, a halogen, hydroxyl, and NH₂;
R⁸ is selected from H, a C₁-C₈ alkyl which is optionally substituted with one or two —NRR or —OR, a C₁-C₈ alkylene-C(O)OR, and a C₁-C₈ alkylene-SO₂R;
R⁹ is H;
R¹⁰ is selected from H, a halogen, and a C₁-C₈ alkyl, which optionally substituted with one or two —OR;
R¹¹ is selected from H, a C₁-C₈ alkyl, —OR, and a halogen;
R¹² is —C(O)N(R)₂ or —C(O)NHR;
R¹³ is H;
wherein:
each R is independently selected from H, a C₁-C₈ alkyl, or a C₁-C₈ haloalkyl, or
two R join to form, together with the atom or atoms to which they are bound, a —C₃-C₁₀ cycloalkyl or 3-10 membered heterocycle, which contains one, two, or three atoms selected from N, O and S; and
wherein the —C₃-C₁₀ cycloalkyl and the 3-10 membered heterocycle is optionally substituted with one or more substituents each independently selected from a C₁-C₈ alkyl, hydroxy, a C₁-C₈ alkoxy, a —(C₃-C₁₀) cycloalkyl, a 3-10 membered heterocycle, a halogen, and a nitrile.

31. The immunogenic composition of claim 10, wherein the STING agonist is: 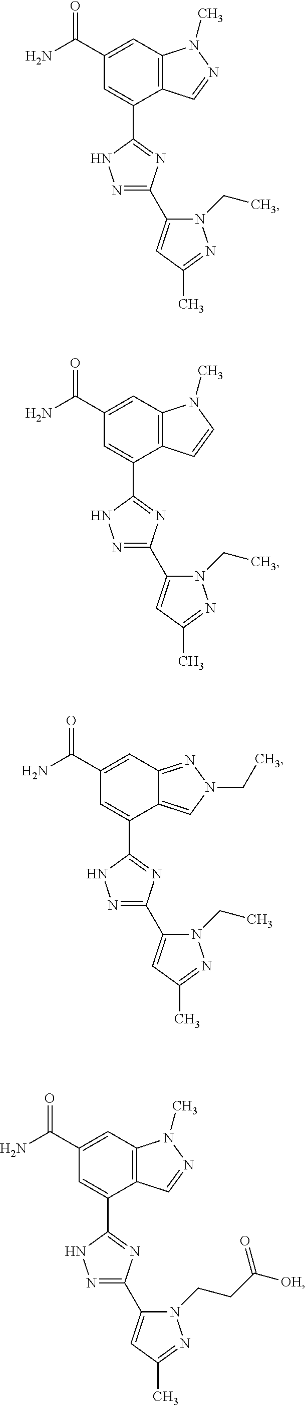 [SMILES: CCn1nc(C)cc1-c1n[nH]c(-c2cc(C(N)=O)cc3c2cnn3C)n1 ; CCn1nc(C)cc1-c1n[nH]c(-c2cc(C(N)=O)cc3c2ccn3C)n1 ; CCn1cc2c(-c3nc(-c4cc(C)nn4CC)n[nH]3)cc(C(N)=O)cc2n1 ; Cc1cc(-c2n[nH]c(-c3cc(C(N)=O)cc4c3cnn4C)n2)n(CCC(=O)O)n1] 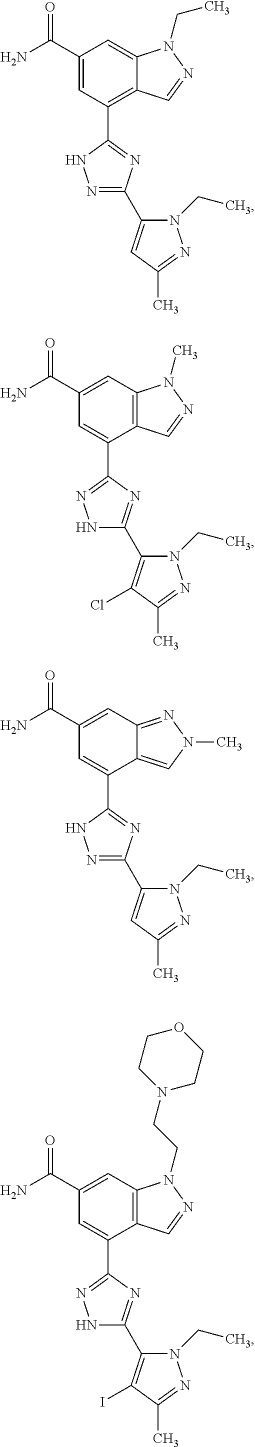 [SMILES: CCn1nc(C)cc1-c1n[nH]c(-c2cc(C(N)=O)cc3c2cnn3CC)n1 ; CCn1nc(C)c(Cl)c1-c1nc(-c2cc(C(N)=O)cc3c2cnn3C)n[nH]1 ; CCn1nc(C)cc1-c1n[nH]c(-c2cc(C(N)=O)cc3nn(C)cc23)n1 ; CCn1nc(C)c(I)c1-c1nc(-c2cc(C(N)=O)cc3c2cnn3CCN2CCOCC2)n[nH]1] 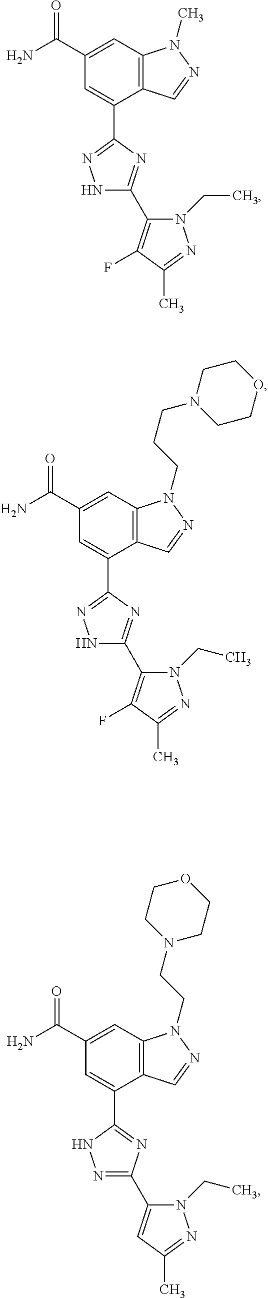 [SMILES: CCn1nc(C)c(F)c1-c1nc(-c2cc(C(N)=O)cc3c2cnn3C)n[nH]1 ; CCn1nc(C)c(F)c1-c1nc(-c2cc(C(N)=O)cc3c2cnn3CCCN2CCOCC2)n[nH]1 ; CCn1nc(C)cc1-c1n[nH]c(-c2cc(C(N)=O)cc3c2cnn3CCN2CCOCC2)n1] 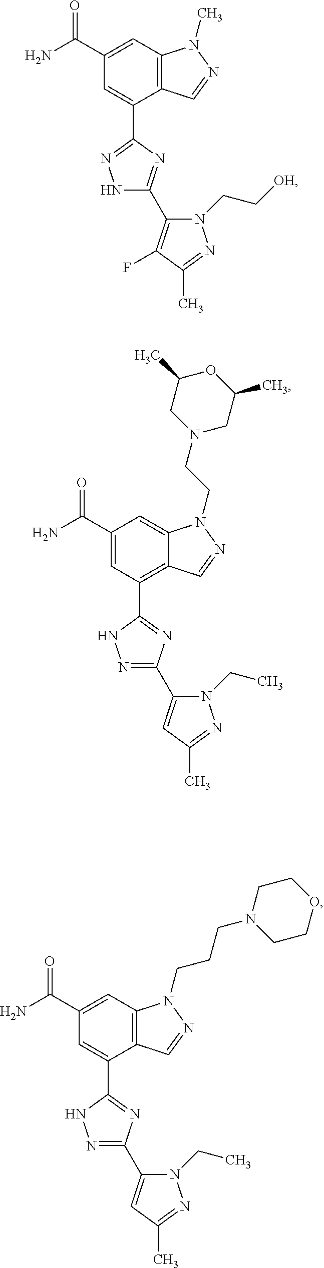 [SMILES: Cc1nn(CCO)c(-c2nc(-c3cc(C(N)=O)cc4c3cnn4C)n[nH]2)c1F ; CCn1nc(C)cc1-c1n[nH]c(-c2cc(C(N)=O)cc3c2cnn3CCN2C[C@@H](C)O[C@@H](C)C2)n1 ; CCn1nc(C)cc1-c1n[nH]c(-c2cc(C(N)=O)cc3c2cnn3CCCN2CCOCC2)n1] 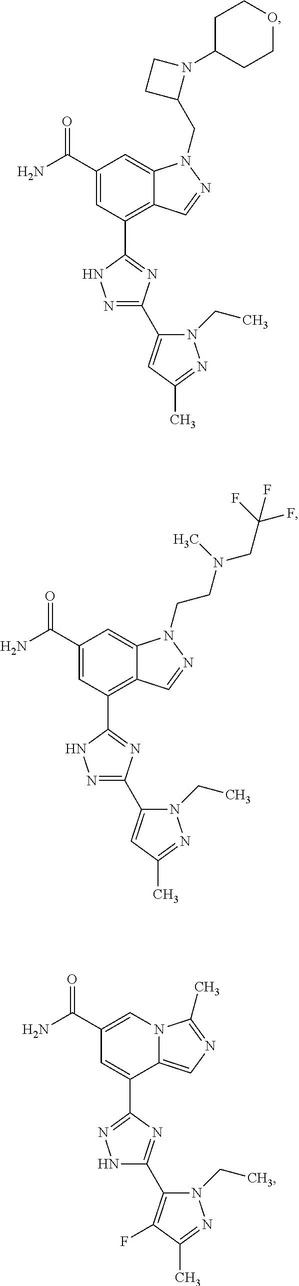 [SMILES: CCn1nc(C)cc1-c1n[nH]c(-c2cc(C(N)=O)cc3c2cnn3CC2CCN2C2CCOCC2)n1 ; CCn1nc(C)cc1-c1n[nH]c(-c2cc(C(N)=O)cc3c2cnn3CCN(C)CC(F)(F)F)n1 ; CCn1nc(C)c(F)c1-c1nc(-c2cc(C(N)=O)cn3c(C)ncc23)n[nH]1] 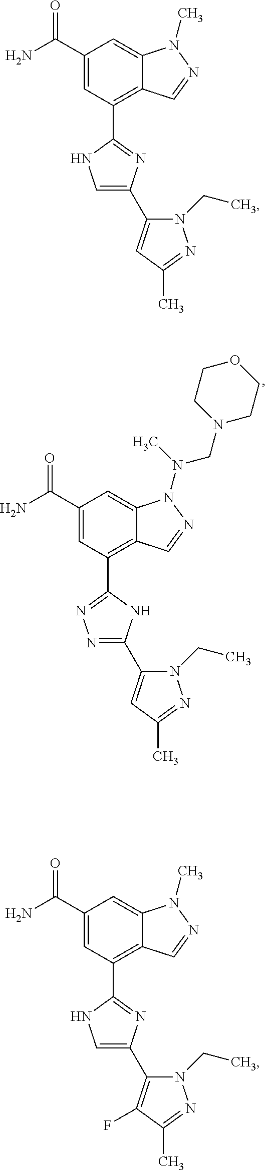 [SMILES: CCn1nc(C)cc1-c1c[nH]c(-c2cc(C(N)=O)cc3c2cnn3C)n1 ; CCn1nc(C)cc1-c1nnc(-c2cc(C(N)=O)cc3c2cnn3N(C)CN2CCOCC2)[nH]1 ; CCn1nc(C)c(F)c1-c1c[nH]c(-c2cc(C(N)=O)cc3c2cnn3C)n1] 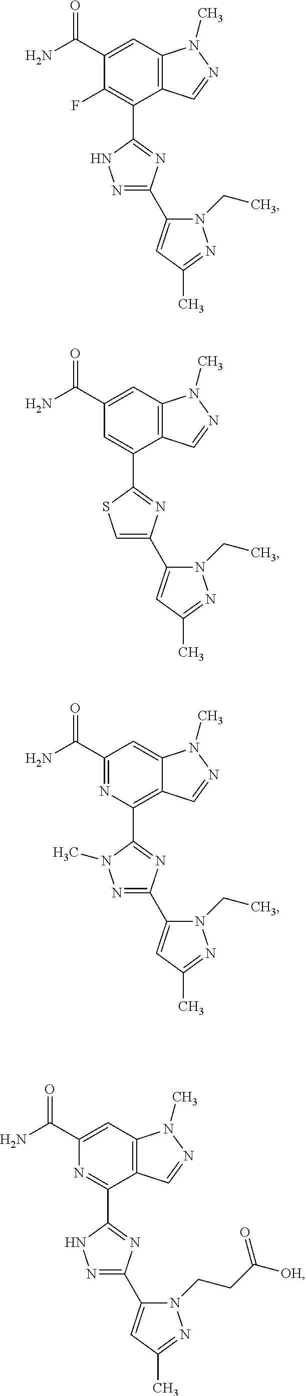 [SMILES: CCn1nc(C)cc1-c1n[nH]c(-c2c(F)c(C(N)=O)cc3c2cnn3C)n1 ; CCn1nc(C)cc1-c1csc(-c2cc(C(N)=O)cc3c2cnn3C)n1 ; CCn1nc(C)cc1-c1nc(-c2nc(C(N)=O)cc3c2cnn3C)n(C)n1 ; Cc1cc(-c2n[nH]c(-c3nc(C(N)=O)cc4c3cnn4C)n2)n(CCC(=O)O)n1] 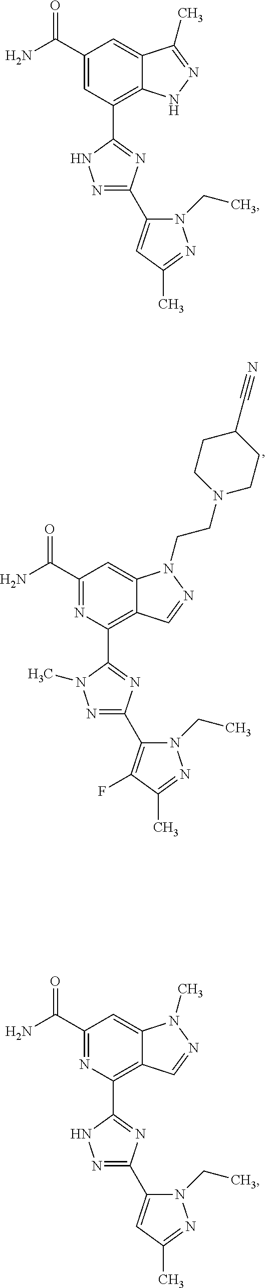 [SMILES: CCn1nc(C)cc1-c1n[nH]c(-c2cc(C(N)=O)cc3c(C)n[nH]c23)n1 ; CCn1nc(C)c(F)c1-c1nc(-c2nc(C(N)=O)cc3c2cnn3CCN2CCC(C#N)CC2)n(C)n1 ; CCn1nc(C)cc1-c1n[nH]c(-c2nc(C(N)=O)cc3c2cnn3C)n1] 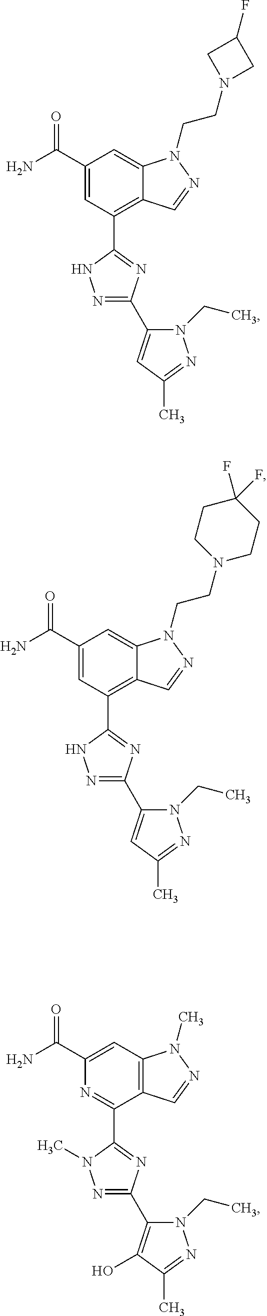 [SMILES: CCn1nc(C)cc1-c1n[nH]c(-c2cc(C(N)=O)cc3c2cnn3CCN2CC(F)C2)n1 ; CCn1nc(C)cc1-c1n[nH]c(-c2cc(C(N)=O)cc3c2cnn3CCN2CCC(F)(F)CC2)n1 ; CCn1nc(C)c(O)c1-c1nc(-c2nc(C(N)=O)cc3c2cnn3C)n(C)n1] 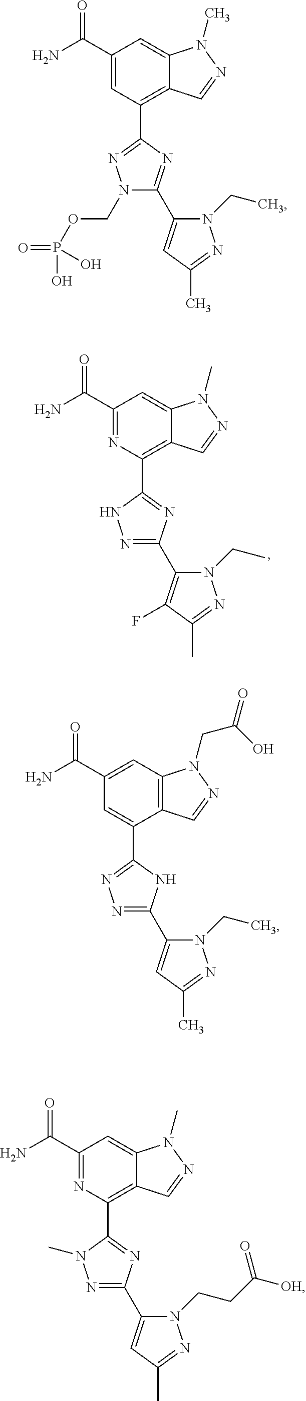 [SMILES: CCn1nc(C)cc1-c1nc(-c2cc(C(N)=O)cc3c2cnn3C)nn1COP(=O)(O)O ; CCn1nc(C)c(F)c1-c1n[nH]c(-c2nc(C(N)=O)cc3c2cnn3C)n1 ; CCn1nc(C)cc1-c1nnc(-c2cc(C(N)=O)cc3c2cnn3CC(=O)O)[nH]1 ; Cc1cc(-c2nc(-c3nc(C(N)=O)cc4c3cnn4C)n(C)n2)n(CCC(=O)O)n1] 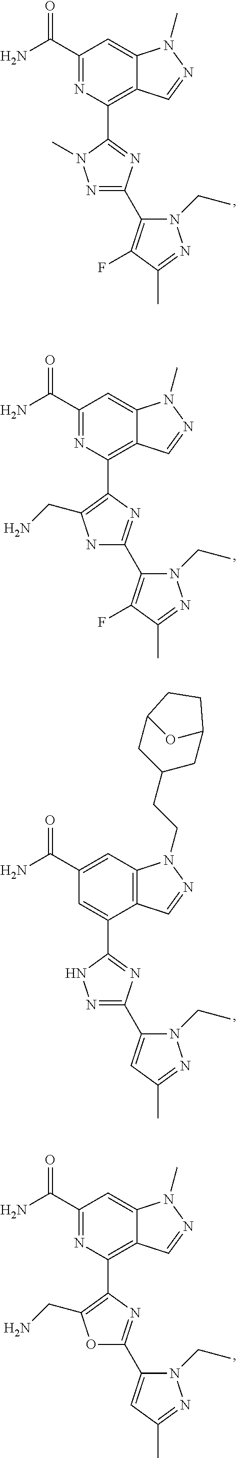 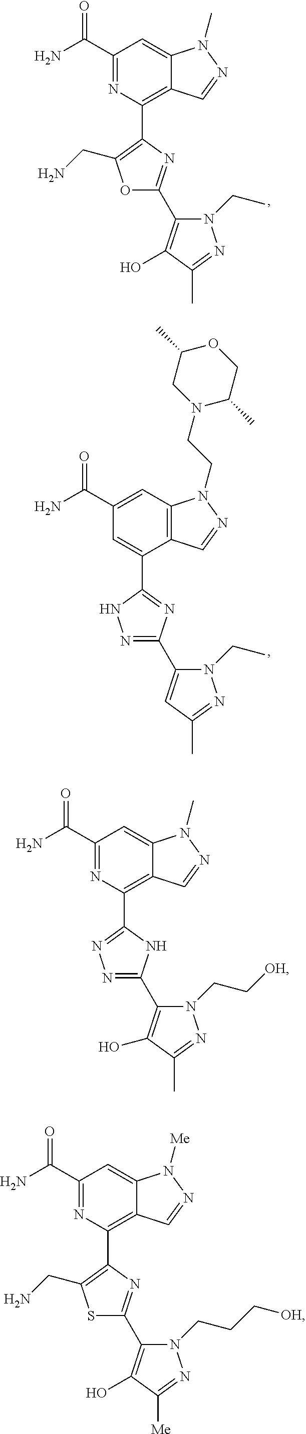 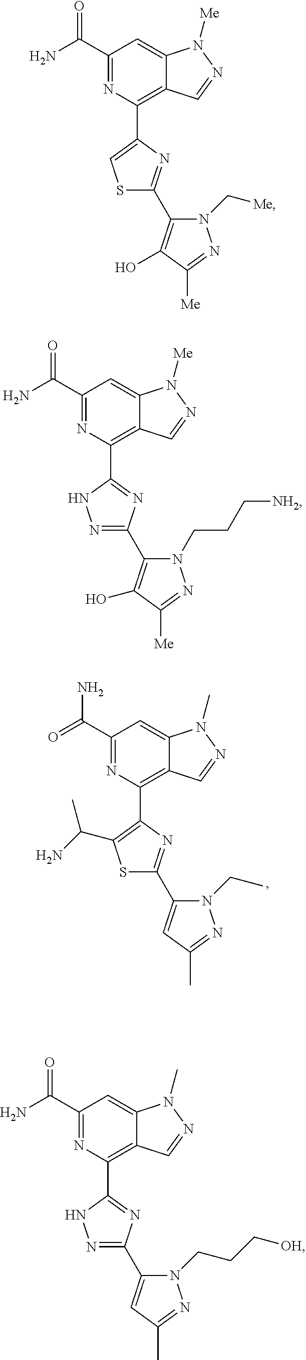 [SMILES: CCn1nc(C)c(O)c1-c1nc(-c2nc(C(N)=O)cc3c2cnn3C)cs1 ; Cc1nn(CCCN)c(-c2n[nH]c(-c3nc(C(N)=O)cc4c3cnn4C)n2)c1O ; CCn1nc(C)cc1-c1nc(-c2nc(C(N)=O)cc3c2cnn3C)c(C(C)N)s1 ; Cc1cc(-c2n[nH]c(-c3nc(C(N)=O)cc4c3cnn4C)n2)n(CCCO)n1] 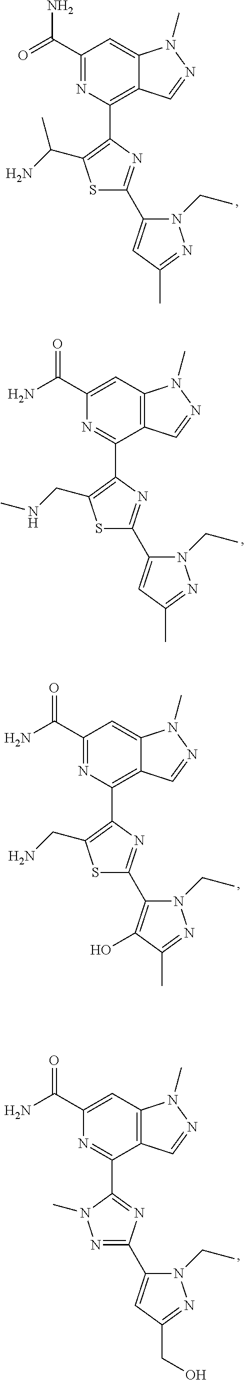 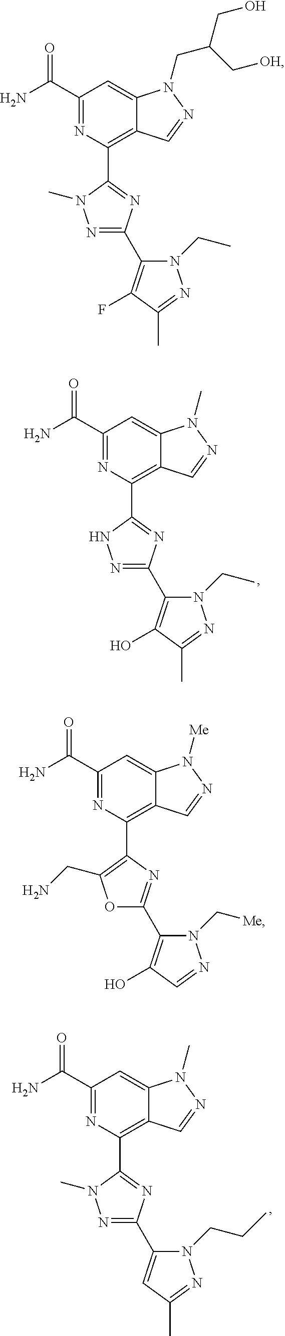 [SMILES: CCn1nc(C)c(F)c1-c1nc(-c2nc(C(N)=O)cc3c2cnn3CC(CO)CO)n(C)n1 ; CCn1nc(C)c(O)c1-c1n[nH]c(-c2nc(C(N)=O)cc3c2cnn3C)n1 ; CCn1ncc(O)c1-c1nc(-c2nc(C(N)=O)cc3c2cnn3C)c(CN)o1 ; CCCn1nc(C)cc1-c1nc(-c2nc(C(N)=O)cc3c2cnn3C)n(C)n1] 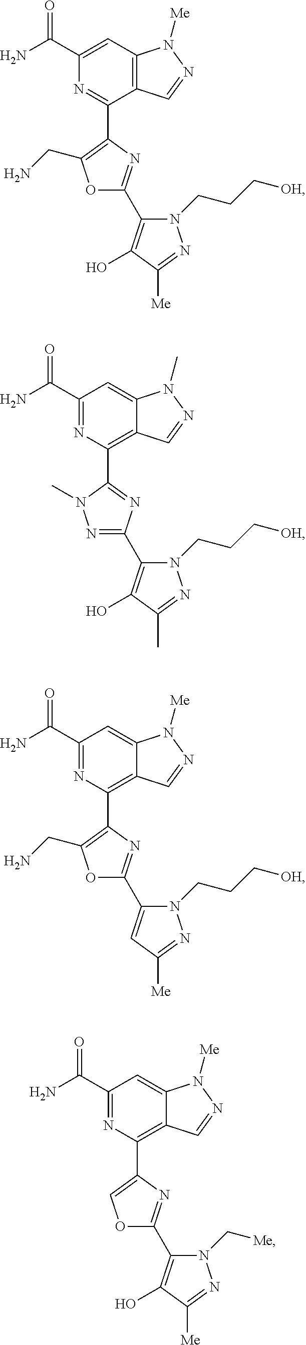 [SMILES: Cc1nn(CCCO)c(-c2nc(-c3nc(C(N)=O)cc4c3cnn4C)c(CN)o2)c1O ; Cc1nn(CCCO)c(-c2nc(-c3nc(C(N)=O)cc4c3cnn4C)n(C)n2)c1O ; Cc1cc(-c2nc(-c3nc(C(N)=O)cc4c3cnn4C)c(CN)o2)n(CCCO)n1 ; CCn1nc(C)c(O)c1-c1nc(-c2nc(C(N)=O)cc3c2cnn3C)co1] 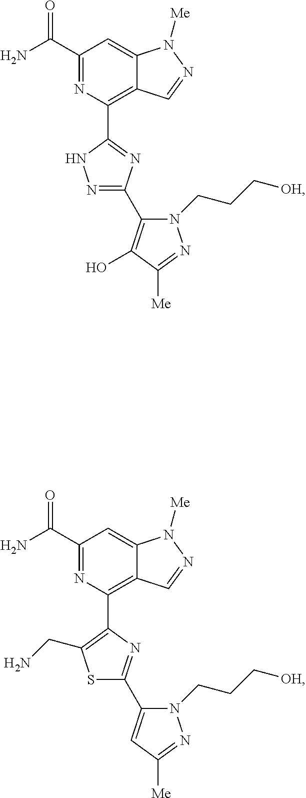 [SMILES: Cc1nn(CCCO)c(-c2n[nH]c(-c3nc(C(N)=O)cc4c3cnn4C)n2)c1O ; Cc1cc(-c2nc(-c3nc(C(N)=O)cc4c3cnn4C)c(CN)s2)n(CCCO)n1]
a pharmaceutically acceptable salt thereof, a tautomer thereof, or any combination thereof.

32. The immunogenic composition of claim 10, wherein:
the STING agonist is a compound of Formula C, a pharmaceutically acceptable salt thereof, a tautomer thereof, or any combination thereof; and
Formula C is: 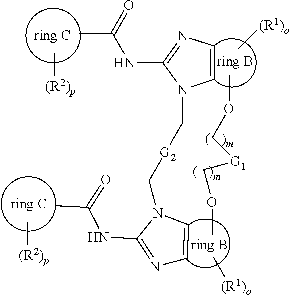
wherein:
G₁ is independently selected from ring A and 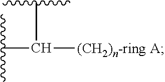
ring A is independently selected from an heterocyclyl, which is optionally substituted with 1 to 4 substituents independently selected from oxo (=0), a halogen, a nitrile, an alkyl, a perhaloalkyl, —OR⁴, —C(═O)OH, —OP(O)(OR⁴)₂, —P(O)(OR⁴)₂, —P(O)(OR⁴)R^{4a}, —SO₂R^{4a}, —SO₂NH₂, —C(═O)N(H)R⁴, a —C(═O)N(alkyl)R⁴, —N(H)C(═O)R^{4a}, —N(H)R⁴, and a —N(alkyl)R⁴, and a heteroaryl, which is optionally substituted with 1 to 4 substituents selected from a halogen, a nitrile, an alkyl, a perhaloalkyl, a —O-alkyl, a —O— perhaloalkyl, a —N(alkyl)alkyl, —N(H)R⁴, a —SO₂-alkyl, a —N(alkyl)C(═O)alkyl, a —N(H)C(═O)alkyl, a —C(═O)N(alkyl)alkyl, a —C(═O)N(H)alkyl, —C(═O)NH₂, a —SO₂N(alkyl)alkyl, a —SO₂N(H)alkyl, a —SO₂NH₂, —C(═O)OH, —OP(O)(OR⁴)₂, —P(O)(OR⁴)₂, and —P(O)(OR⁴)R^{4a},
ring B is an aromatic carbocyclic ring;
ring C is a five-membered heteroaryl, which is optionally substituted with 1 to 4 substituents selected from a halogen, a nitrile, an alkyl, a perhaloalkyl, a —O-alkyl, a —O-perhaloalkyl, a —N(alkyl)alkyl, —N(H)R⁴, a —SO₂-alkyl, a —N(alkyl)C(═O)alkyl, a —N(H)C(═O)alkyl, a —C(═O)N(alkyl)alkyl, a —C(═O)N(H)alkyl, —C(═O)NH₂, a —SO₂N(alkyl)alkyl, a —SO₂N(H)alkyl, a —SO₂NH₂, —C(═O)OH, —OP(O)(OR⁴)₂, —P(O)(OR⁴)₂, and —P(O)(OR⁴)R^{4a};
R¹ is —CON(R³)₂;
R² is independently selected from hydrogen; a C₁-C₆ alkyl, which is optionally substituted with 1 to 4 substituents independently selected from a halogen, an alkyl, a perhaloalkyl, a cycloalkyl, a heterocyclyl, —N(R⁴)₂, and —OR⁴; and a optionally-substitute C₃-C₅ monocyclic cycloalkyl, which is optionally substituted with 1 to 4 substituents independently selected from a halogen, an alkyl, a perhaloalkyl, —N(R⁴)₂, and —OR⁴;
R³ is independently selected from hydrogen and a C₁-C₆ alkyl, which is optionally substituted with 1 to 4 substituents independently selected from a halogen, an alkyl, a perhaloalkyl, a cycloalkyl, a heterocyclyl, —N(R⁴)₂, and —OR⁴;
m is selected from 0 and 1;
n is selected from 0, 1, and 2;
o is 1;
p is selected from 0, 1, and 2;
each R⁴ is independently selected from hydrogen, an alkyl, and a cycloalkyl; and
each R^{4a} is independently selected from an alkyl and a cycloalkyl.

33. The immunogenic composition of claim 10, wherein the STING agonist is: 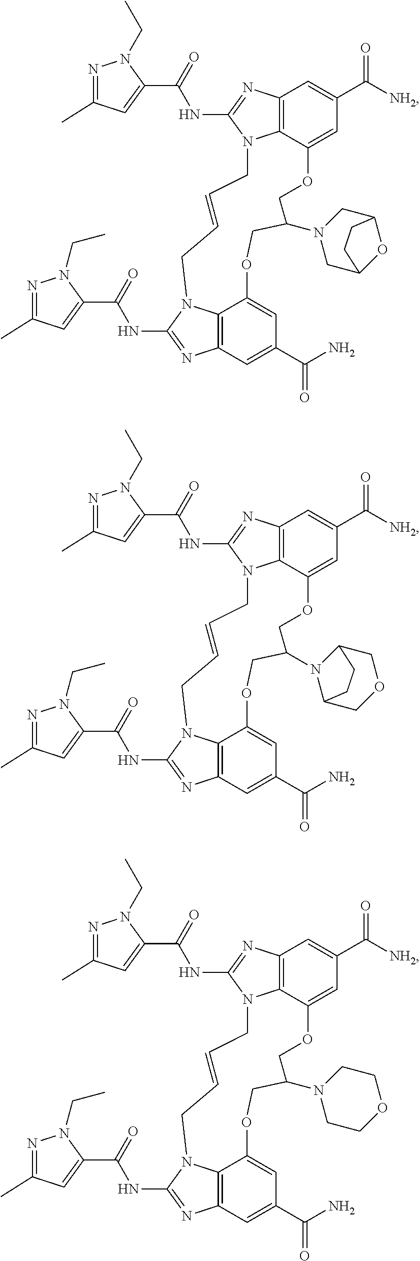 [SMILES: CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CC2CCC(C1)O2)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1C2CCC1COC2)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCOCC1)CO3] 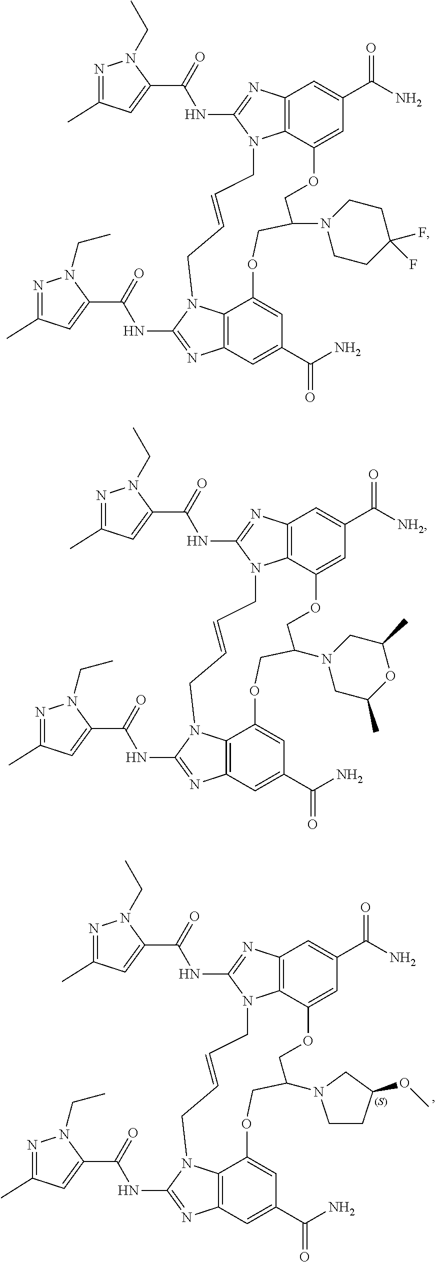 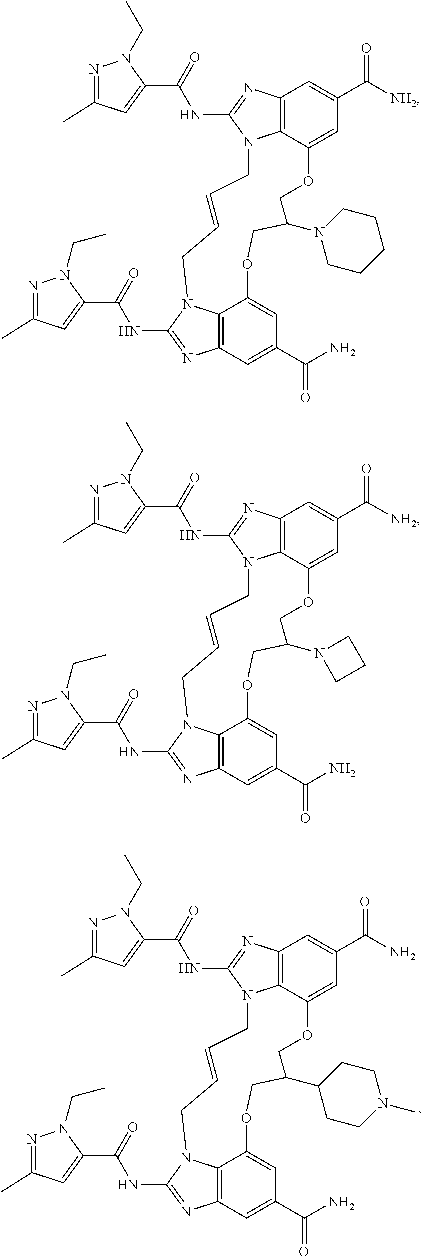 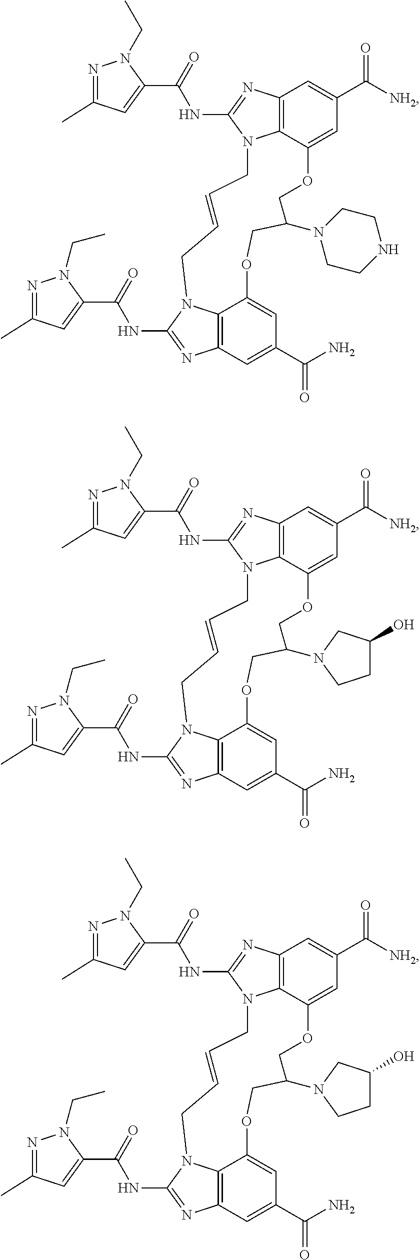 [SMILES: CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCNCC1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CC[C@H](O)C1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CC[C@@H](O)C1)CO3] 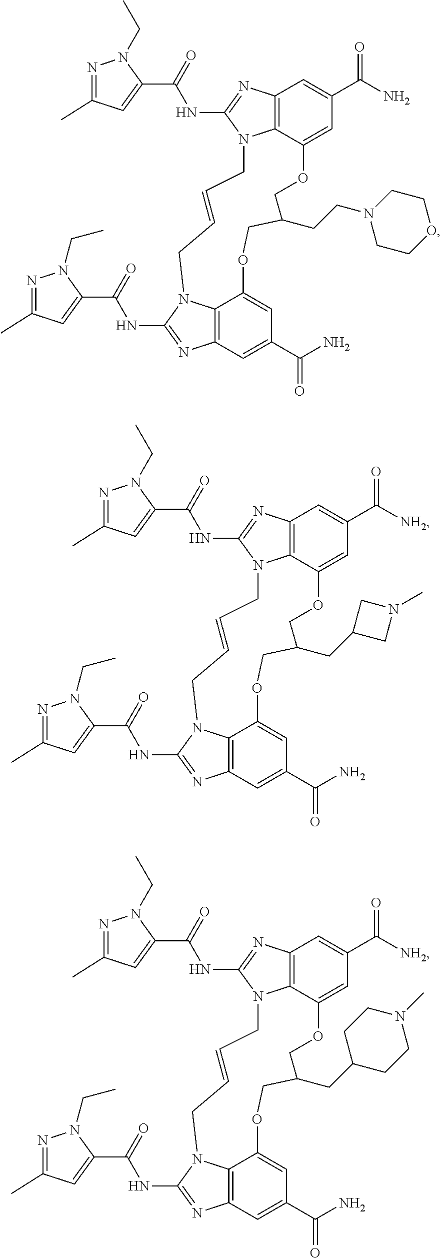 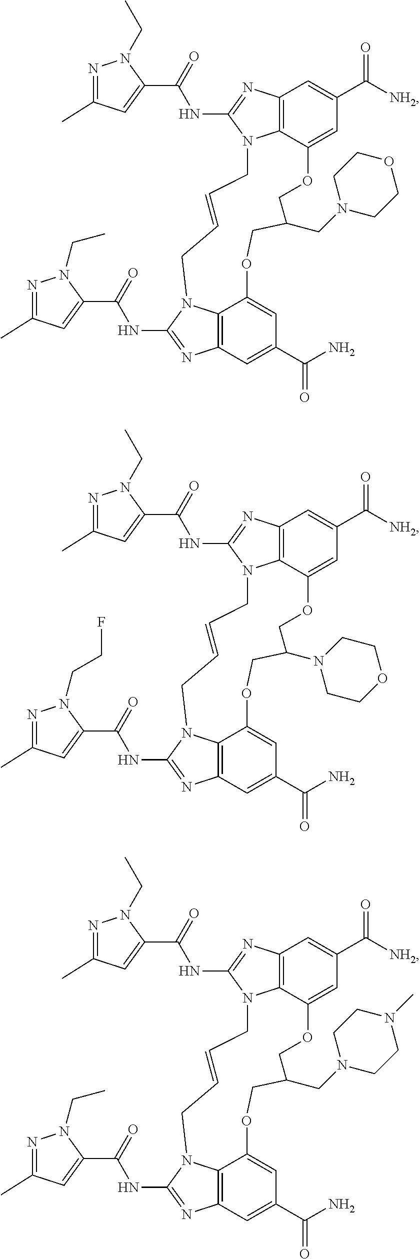 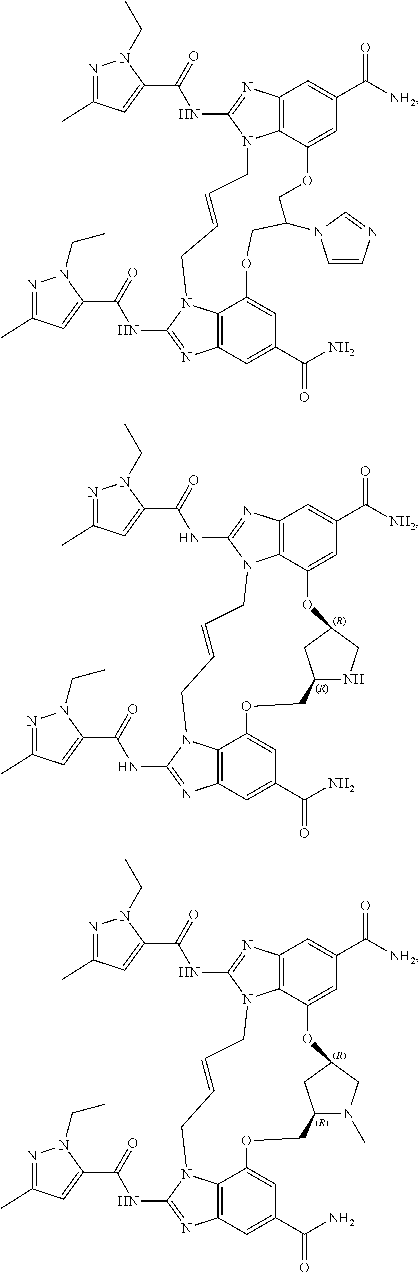 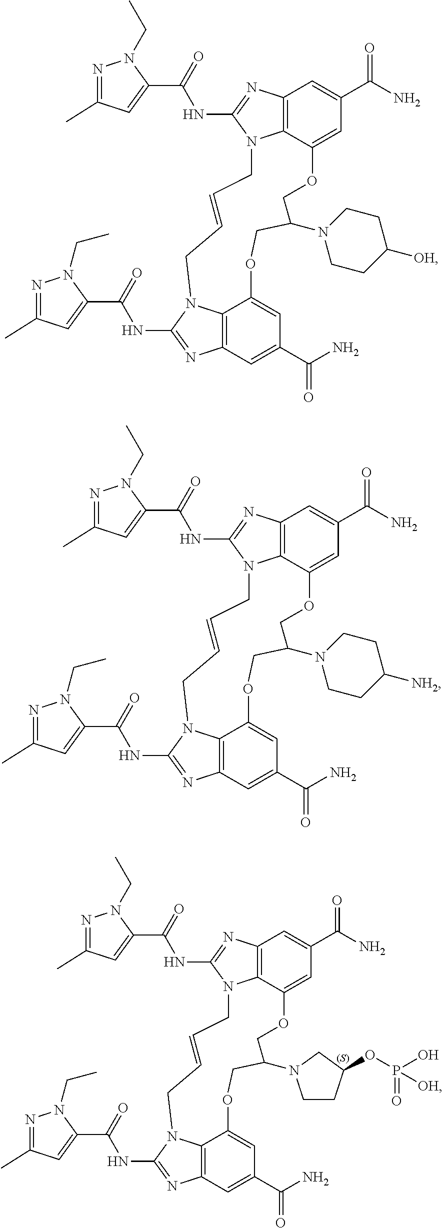 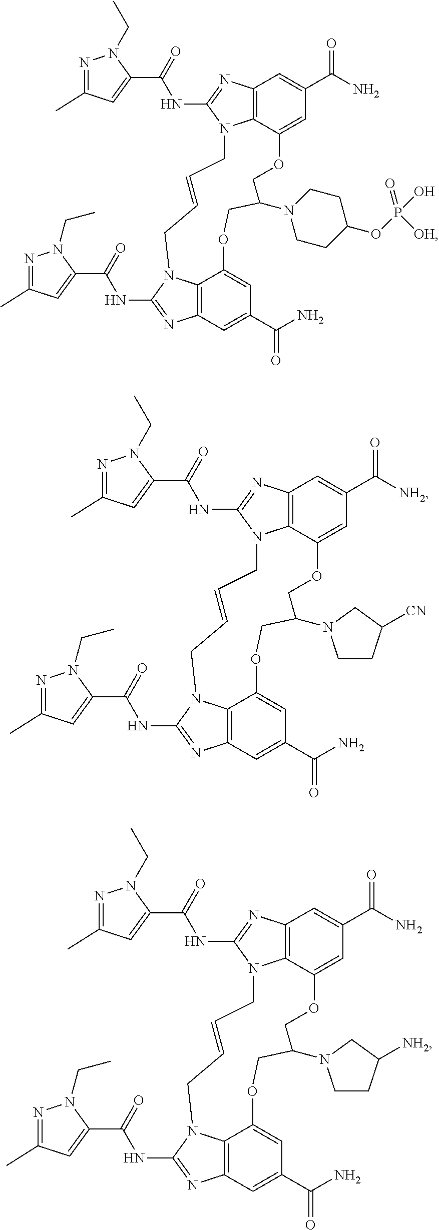 [SMILES: CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCC(OP(=O)(O)O)CC1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCC(C#N)C1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCC(N)C1)CO3] 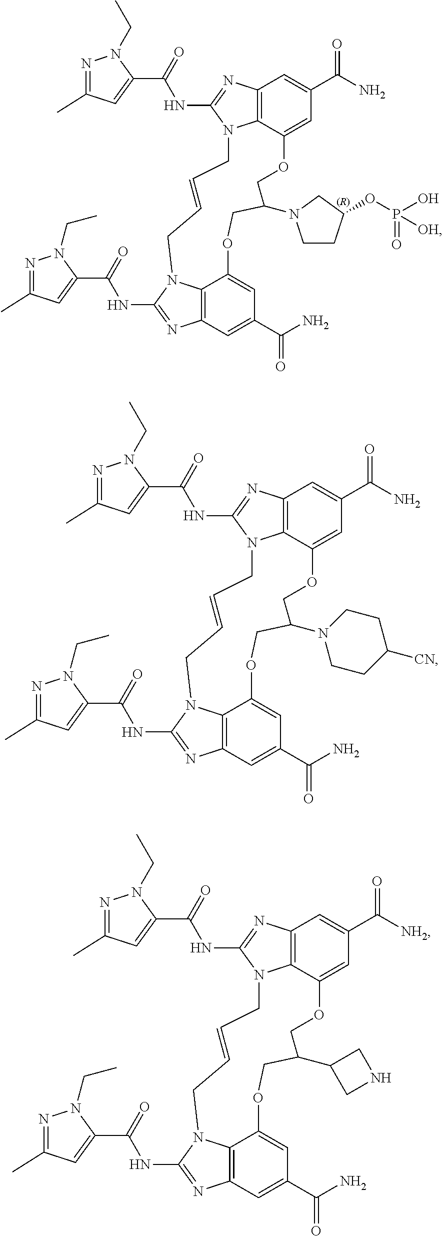 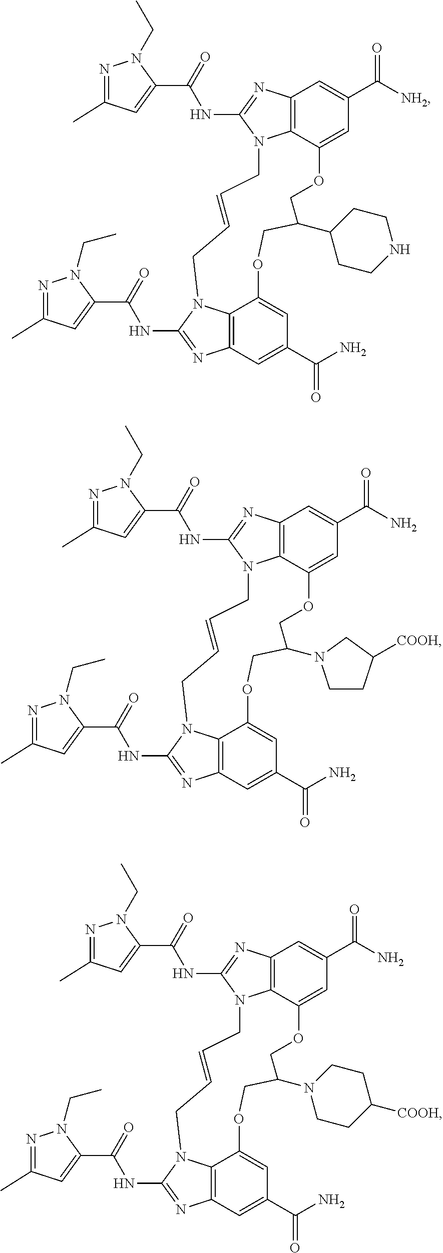 [SMILES: CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(C1CCNCC1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCC(C(=O)O)C1)CO3 ; CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC(N1CCC(C(=O)O)CC1)CO3] 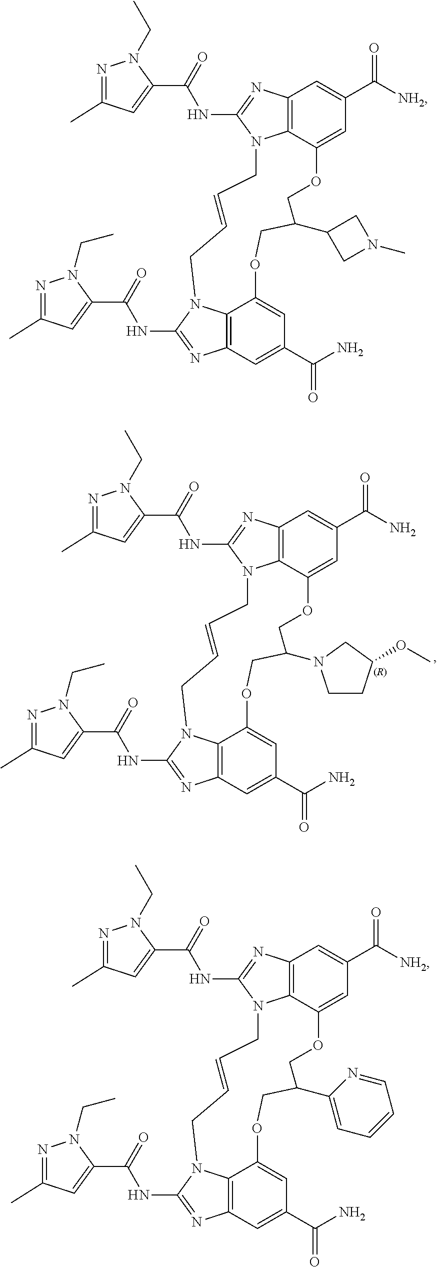
a pharmaceutically acceptable salt thereof, a tautomer thereof, or any combination thereof.

34. The immunogenic composition of claim 10, wherein the STING agonist is: IMSA101, ADU-S100 (MIW815), BMS-986301, CRD5500, CMA 10-carboxymethyl-9-acridanone), diABZI STING agonist-1 (CAS No.: 2138299-34-8), DMXAA (ASA404/vadimezan), 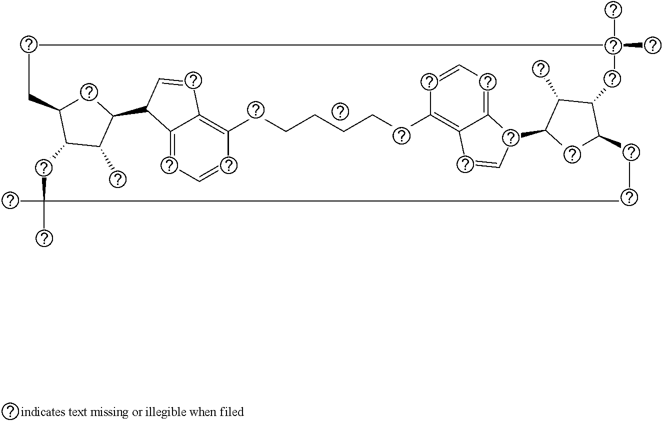
(E7766, Cas no. 2242635-02-3), MK-1454, MK-2118, SB-11285, SRCB-0074, TAK-676, TTI-10001, or a pharmaceutically acceptable salt thereof.

35. The immunogenic composition of claim 10, wherein:
the STING agonist is a compound according to Formula (I-N), a pharmaceutically acceptable salt thereof, a tautomer thereof, or any combination thereof; and
Formula (I-N) is: 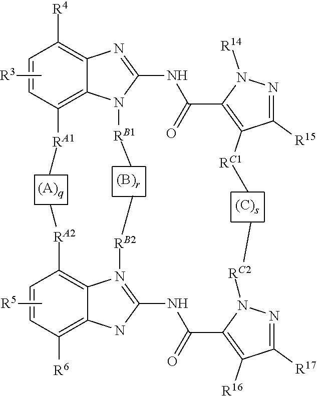
wherein:
q is 0 or 1;
r is 0 or 1;
s is 0 or 1;
q+r+s=1 or 2;
when q is 0, R^{A1} and R^{A2} are each independently: hydrogen, a halogen, hydroxy, —O—P(O)(OH)₂, —O—(O)(R^{I}R^{II})₂, —N(R^{e})(R^{f}), —CO₂R^{f}, —N(R^{f})COR^{b}, a —N(R^{g})SO₂(C₁-C₄alkyl)-N(R^{e})(R^{f}), or a —N(R^{g})CO(C₁-C₄alkyl)-N(R^{h})(R^{f}),
a first optionally substituted (C₁-C₆alkyl), a first optionally substituted (C₁-C₆alkyl)oxy-, a first optionally substituted (C₁-C₆alkyl)amino-, or a first optionally substituted (C₁-C₆alkyl)(C₁-C₄alkyl)amino-, wherein the (C₁-C₆alkyl) of the first optionally substituted (C₁-C₆alkyl), the first optionally substituted (C₁-C₆alkyl)oxy-, the first optionally substituted (C₁-C₆alkyl)amino-, and the first optionally substituted (C₁-C₆alkyl)(C₁-C₄alkyl)amino- are each optionally substituted by 1-4 substituents each independently selected from hydroxy, —O—P(O)(OH)₂, —O—P(O)(R^{I}R^{II})₂, C₁-C₄alkoxy-, —N(R^{e})(R^{f}), —CO₂(R^{f}), —CON(R^{e})(R^{f}), a first optionally substituted phenyl, a first optionally substituted 5-6 membered heterocycloalkyl, and a first optionally substituted 5-6 membered heteroaryl group, wherein the first optionally substituted phenyl, the first optionally substituted 5-6 membered heterocycloalkyl, and the first optionally substituted 5-6 membered heteroaryl are each optionally substituted by 1-4 substituents each independently selected from:
a halogen, hydroxy, —O—P(O)(OH)₂, —O—P(O)(R^{I}R^{II})₂, amino, a (C₁-C₆alkyl)amino-, a (C₁-C₆alkyl)(C₁-C₆alkyl)amino-, a —(C₁-C₆alkyl)-NH₂, a halo(C₁-C₆alkyl), a hydroxy-(C₁-C₄alkyl)-, a —(C₁-C₄alkyl)-O—P(O)(OH)₂, a —(C₁-C₄alkyl)-O—P(O)(R^{I}R^{II})₂, a halo(C₁-C₄alkoxy)-, a C₁-C₄alkoxy-, a hydroxy-(C₂-C₄alkoxy)-, a —(C₂-C₄alkoxy)-O—P(O)(OH)₂, a —(C₂-C₄alkoxy)-O—P(O)(R^{I}R^{II})₂, a —C₁-C₄alkyl-(C₁-C₄alkoxy), and a C₁-C₄alkoxy-(C₁-C₄alkoxy)-;
when r is 0, R^{B1} and R^{B2} are each independently: hydrogen, a second optionally substituted C₁-C₆alkyl, a halo(C₁-C₆alkyl), a first optionally substituted C₂-C₆alkenyl, an optionally substituted C₂-C₆alkynyl, a first optionally substituted C₃-C₆cycloalkyl, a first optionally substituted 4-6 membered heterocycloalkyl, a second optionally substituted phenyl, a second optionally substituted 5-6 membered heteroaryl, or a first optionally substituted 9-10 membered heteroaryl, wherein the second optionally substituted C₁-C₆alkyl, the first optionally substituted C₂-C₆alkenyl, the optionally substituted C₂-C₆alkynyl, the first optionally substituted C₃-C₆cycloalkyl, the first optionally substituted 4-6 membered heterocycloalkyl, the second optionally substituted phenyl, the second optionally substituted 5-6 membered heteroaryl, and the first optionally substituted 9-10 membered heteroaryl are each independently and optionally substituted by 1-4 substituents each independently selected from:
a halogen, a nitro, —R^{c}, —OH, —O—P(O)(OH)₂, —O—P(O)(R^{I}R^{II})₂, —OR^{c}, —NH₂, —NR^{c}R^{c}, —NR^{c}R^{d}, —OCOR^{c}, —CO₂H, —CO₂R^{c}, —SOR^{c}, —SO₂R^{c}, —CONH₂, —CONR^{c}R^{d}, —SO₂NH₂, —SO₂NR^{c}R^{d}, —OCONH₂, —OCONR^{c}R^{d}, —NR^{d}COR^{c}, —NR^{d}SOR^{c}, —NR^{d}CO₂R^{c}, and —NR^{d}SO₂R^{c};
when s is 0:
R^{C1} is hydrogen, a halogen, or a C₁-C₄alkyl and
R^{C2} is a C₁-C₄alkyl, which is optionally substituted by a substituent selected from:
—OR^{c}, —NR^{c}R^{d}, —CO₂R^{c}, —CONR^{c}R^{d}, —SO₂NR^{c}R^{d}, and —OCONR^{c}R^{d};
when q is 1
R^{A1} and R^{A2} are each independently: —CH₂—, —NR^{e}—, or —O—, and
A, taken together with R^{A1} and R^{A2}, forms a linking group, wherein A is: a -halo(C₁-C₁₂alkyl)-, a first optionally substituted —C₁-C₁₂alkyl-, a first optionally substituted —C₂-C₁₂alkenyl-, a first optionally substituted —C₂-C₁₂alkynyl-, a first optionally substituted —C₁-C₆alkyl-O—C₁-C₆alkyl-, a first optionally substituted —C₁-C₆alkyl-NR^{a}—C₁-C₆alkyl-, a first optionally substituted —C₁-C₆alkyl-(C₃-C₆cycloalkyl)-C₁-C₆alkyl-, a first optionally substituted —C₁-C₆alkyl-phenyl-C₁-C₆alkyl-, a first optionally substituted —C₁-C₆alkyl-(4-6 membered heterocycloalkyl)-C₁-C₆alkyl-, or a first optionally substituted —C₁-C₆alkyl-(5-6 membered heteroaryl)-C₁-C₆alkyl-, wherein:
the alkyl moiety of the first optionally substituted —C₁-C₁₂alkyl-, the first optionally substituted —C₂-C₁₂alkenyl-, the first optionally substituted —C₂-C₁₂alkynyl-, the first optionally substituted —C₁-C₆alkyl-O—C₁-C₆alkyl-, the first optionally substituted —C₁-C₆alkyl-NR^{a}—C₁-C₆alkyl-, the first optionally substituted —C₁-C₆alkyl-(C₃-C₆cycloalkyl)-C₁-C₆alkyl-, the first optionally substituted —C₁-C₆alkyl-phenyl-C₁-C₆alkyl-, the first optionally substituted —C₁-C₆alkyl-(4-6 membered heterocycloalkyl)-C₁-C₆alkyl-, and the first optionally substituted —C₁-C₆alkyl-(5-6 membered heteroaryl)-C₁-C₆alkyl- are each optionally substituted by 1-4 substituents each independently selected from:
a halogen, halo(C₁-C₄alkyl), —OH, —O—P(O)(OH)₂, —O—P(O)(R^{I}R^{II})₂, —OR^{c}, —NH₂, —NR^{c}R^{d}, —OCOR^{c}, —CO₂H, —CO₂R^{c}, —SOR^{c}, —SO₂R^{c}, —CONH₂, —CONR^{c}R^{d}, —SO₂ NH₂, —SO₂NR^{c}R^{d}, —OCONH₂, —OCONR^{c}R^{d}, —NR^{d}CO R^{c}, —NR^{d}SOR^{c}, —NR^{d}CO₂R^{c}, and —NR^{d}SO₂R^{c}, and the C₃-C₆cycloalkyl moiety of the first optionally substituted —C₁-C₆alkyl-(C₃-C₆cycloalkyl)-C₁-C₆alkyl-, the phenyl moiety of the first optionally substituted C₁-C₆alkyl-phenyl-C₁-C₆alkyl-, the 4-6 membered heterocycloalkyl moiety of the first optionally substituted —C₁-C₆alkyl-(4-6 membered heterocycloalkyl)-C₁-C₆alkyl-, and the 5-6 membered heteroaryl moiety of the first optionally substituted —C₁-C₆alkyl-(5-6 membered heteroaryl)-C₁-C₆alkyl- are each independently and optionally substituted by 1-4 substituents each independently selected from:
a halogen, a hydroxy, —O—P(O)(OH)₂, —O—P(O)(R^{I}R^{II})₂, an amino, a (C₁-C₄alkyl)amino-, a (C₁-C₄alkyl)(C₁-C₄alkyl)amino-, a C₁-C₄alkyl, a halo(C₁-C₄alkyl), a halo(C₁-C₄alkoxy)-, a C₁-C₄alkoxy-, a hydroxy-(C₁-C₄alkoxy)-, a —(C₁-C₄alkoxyl)-O—P(O)(OH)₂, a —(C₁-C₄alkoxyl)-O—P(O)(R^{I}R^{II})₂, and a C₁-C₄alkoxy-(C₁-C₄alkoxy)-;
when r is 1:
R^{B1} and R^{B2} are each independently —CH₂— and
B, taken together with R^{B1} and R^{B2}, forms a linking group,
wherein B is a bond or B is a -halo(C₁-C₁₀alkyl)-, an optionally substituted —C₁-C₁₀alkyl-, an optionally substituted —C₂-C₁₀alkenyl-, an optionally substituted —C₂-C₁₀alkynyl-, a second optionally substituted —C₁-C₆alkyl-O—C₁-C₆alkyl-, a second optionally substituted —C₁-C₆alkyl-NR^{a}—C₁-C₆alkyl-, a second optionally substituted C₃-C₆cycloalkyl, a third optionally substituted phenyl, a second optionally substituted 4-6 membered heterocycloalkyl, a third optionally substituted 5-6 membered heteroaryl, an optionally substituted —C₁-C₄alkyl-(C₃-C₆cycloalkyl)-C₁-C₄alkyl-, an optionally substituted —C₁-C₄alkyl-phenyl-C₁-C₄alkyl-, an optionally substituted —C₁-C₄alkyl-(4-6 membered heterocycloalkyl)-C₁-C₄alkyl-, or an optionally substituted —C₁-C₄alkyl-(5-6 membered heteroaryl)-C₁-C₄alkyl-, wherein:
the alkyl moiety of the optionally substituted —C₁-C₁₀alkyl-, the optionally substituted —C₂-C₁₀alkenyl-, the optionally substituted —C₂-C₁₀alkynyl-, the second optionally substituted —C₁-C₆alkyl-O—C₁-C₆alkyl-, the second optionally substituted —C₁-C₆alkyl-NR^{a}—C₁-C₆alkyl-, the optionally substituted —C₁-C₄alkyl-(C₃-C₆cycloalkyl)-C₁-C₄alkyl-, the optionally substituted —C₁-C₄alkyl-phenyl-C₁-C₄alkyl-, the optionally substituted —C₁-C₄alkyl-(4-6 membered heterocycloalkyl)-C₁-C₄alkyl-, and the optionally substituted —C₁-C₄alkyl-(5-6 membered heteroaryl)-C₁-C₄alkyl- are each independently and optionally substituted by 1 or 2 substituents each independently selected from:
a halogen, a halo(C₁-C₄alkyl), —OH, —O—P(O)(OH)₂, —O—P(O)(R_{I}R_{II})₂, —OR^{c}, —NH₂, —NR^{c}R^{d}, —OCOR^{c}, —CO₂H, —CO₂R^{c}, —SOR^{c}, —SO₂R^{c}, —CONH₂, —CONR^{c}R^{d}, —SO₂ NH₂, —SO₂NR^{c}R^{d}, —OCONH₂, —OCONR^{c}R^{d}, —NR^{d}CO R^{c}, —NR^{d}SOR^{c}, —NR^{d}CO₂R^{c}, and —NR^{d}SO₂R^{c}, and
the second optionally substituted C₃-C₆cycloalkyl, the third optionally substituted phenyl, the second optionally substituted 4-6 membered heterocycloalkyl, the third optionally substituted 5-6 membered heteroaryl the C₃-C₆cycloalkyl moiety of the optionally substituted —C₁-C₄alkyl-(C₃-C₆cycloalkyl)-C₁-C₄alkyl-, the phenyl moiety of the optionally substituted —C₁-C₄alkyl-phenyl-C₁-C₄alkyl-, the 4-6 membered heterocycloalkyl moiety of the optionally substituted —C₁-C₄alkyl-(4-6 membered heterocycloalkyl)-C₁-C₄alkyl-, and the 5-6 membered heteroaryl moiety of the optionally substituted —C₁-C₄alkyl-(5-6 membered heteroaryl)-C₁-C₄alkyl- are each independently and optionally substituted by 1-4 substituents each independently selected from:
a halogen, a hydroxy, —O—P(O)(OH)₂, —O—P(O)(R^{I}R^{II})₂, amino, a (C₁-C₄alkyl)amino-, a (C₁-C₄alkyl)(C₁-C₄alkyl)amino-, C₁-C₄alkyl, halo(C₁-C₄alkyl), a halo(C₁-C₄alkoxy)-, a C₁-C₄alkoxy-, hydroxy-(C₂-C₄alkoxy)-, a -(C₂-C₄alkoxy)O—P(O)(OH)₂, a —(C₂-C₄alkoxy)-O—P(O)(R^{I}R^{II})₂, and a C₁-C₄alkoxy-(C₁-C₄alkoxy)-;
when s is 1
R^{C1} and R^{C2} are each independently —CH₂— and
C, taken together with R^{C1} and R^{C2}, forms a linking group,
wherein C is: a -halo(C₁-C₁₂alkyl)-, a second optionally substituted —C₁-C₁₂alkyl-, a second optionally substituted —C₂-C₁₂alkenyl-, a second optionally substituted —C₂-C₁₂alkynyl-, a third optionally substituted —C₁-C₆alkyl-O—C₁-C₆alkyl-, a third optionally substituted —C₁-C₆alkyl-NR^{a}—C₁-C₆alkyl-, a second optionally substituted —C₁-C₆alkyl-(C₃-C₆cycloalkyl)-C₁-C₆alkyl-, a second optionally substituted —C₁-C₆alkyl-phenyl-C₁-C₆alkyl-, a second optionally substituted —C₁-C₆alkyl-(4-6 membered heterocycloalkyl)-C₁-C₆alkyl-, or a second optionally substituted —C₁-C₆alkyl-(5-6 membered heteroaryl)-C₁-C₆alkyl-, wherein:
the alkyl moiety of the second optionally substituted —C₁-C₁₂alkyl-, the second optionally substituted —C₂-C₁₂alkenyl-, the second optionally substituted —C₂-C₁₂alkynyl-, the third optionally substituted —C₁-C₆alkyl-O—C₁-C₆alkyl-, the third optionally substituted —C₁-C₆alkyl-NR^{a}—C₁-C₆alkyl-, the second optionally substituted —C₁-C₆alkyl-(C₃-C₆cycloalkyl)-C₁-C₆alkyl-, the second optionally substituted —C₁-C₆alkyl-phenyl-C₁-C₆alkyl-, the second optionally substituted —C₁-C₆alkyl-(4-6 membered heterocycloalkyl)-C₁-C₆alkyl-, and the second optionally substituted —C₁-C₆alkyl-(5-6 membered heteroaryl)-C₁-C₆alkyl- are each independently and optionally substituted by 1 or 2 substituents each independently selected from:
a halogen, a halo(C₁-C₄alkyl), —OH, —O—P(O)(OH)₂, —O—P(O)(R^{I}R^{II})₂, —OR^{c}, —NH₂, —NR^{c}R^{d}, —OCOR⁰, —CO₂H, —CO₂R^{c}, —SOR^{c}, —SO₂R^{c}, —CONH₂, —CONR^{c}R^{d}, —SO₂ NH₂, —SO₂NR^{c}R^{d}, —OCONH₂, —OCONR^{c}R^{d}, —NR^{d}CO R^{c}, —NR^{d}SOR^{c}, —NR^{d}CO₂R, and —NR^{d}SO₂R^{c}, and
the C₃-C₆cycloalkyl moiety of the second optionally substituted —C₁-C₆alkyl-(C₃-C₆cycloalkyl)-C₁-C₆alkyl-, the phenyl moiety of the second optionally substituted —C₁-C₆alkyl-phenyl-C₁-C₆alkyl-, the 4-6 membered heterocycloalkyl moiety of the second optionally substituted —C₁-C₆alkyl-(4-6 membered heterocycloalkyl)-C₁-C₆alkyl-, or the 5-6 membered heteroaryl moiety of the second optionally substituted —C₁-C₆alkyl-(5-6 membered heteroaryl)-C₁-C₆alkyl- are each independently and optionally substituted by 1-4 substituents each independently selected from a halogen, hydroxy, —O—P(O)(OH)₂, —O—P(O)(R^{I}R^{II})₂, an amino, a (C₁-C₄alkyl)amino-, a (C₁-C₄alkyl)(C₁-C₄alkyl)amino-, a C₁-C₄alkyl, a halo(C₁-C₄alkyl), a halo(C₁-C₄alkoxy)-, a C₁-C₄alkoxy-, a hydroxy-(C₂-C₄alkoxy)-, a —(C₂-C₄alkoxy)-O—P(O)(OH)₂, a —(C₂-C₄alkoxy)-O—P(O)(R^{I}R^{II})₂, and a —C₁-C₄alkoxy-(C₁-C₄alkoxy)-;
R³ and R⁵ are each independently —CON(R^{d})(R^{f}), or one of R³ and R⁵ is —CON(R^{d})(R^{f}), and the other of R³ and R⁵ is H, COOH, or —CO₂(R^{c});
R⁴ and R⁶ are each independently selected from hydrogen, a halogen, a halo(C₁-C₆alkyl), a halo(C₁-C₆alkoxy)-, hydroxy, —O—P(O)(OH)₂, —O—P(O)(R^{I}R^{II})₂, —NH₂, —NR^{c}R^{c}, —NR^{c}R^{d}, —COR^{c}, —CO₂R^{c}, —N(R^{d})COR^{c}, —N(R^{d})SO₂R^{c}, —N(R^{g})SO₂(C₁-C₂alkyl)-N(R^{h})(R), —N(R^{g})CO(C₁-C₂alkyl)-N(R^{h})(R^{f}), a second optionally substituted (C₁-C₆alkyl), a second optionally substituted (C₁-C₆alkyl)oxy-, a second optionally substituted (C₁-C₆alkyl)amino-, and a second optionally substituted (C₁-C₆alkyl)(C₁-C₄alkyl)amino-,
wherein the (C₁-C₆alkyl) moiety of the second optionally substituted (C₁-C₆alkyl), the (C₁-C₆alkyl) moiety of the second optionally substituted (C₁-C₆alkyl)oxy-, the (C₁-C₆alkyl) moiety of the second optionally substituted (C₁-C₆alkyl)amino-, and the (C₁-C₆alkyl) moiety of the second optionally substituted (C₁-C₆alkyl)(C₁-C₄alkyl)amino- are each independently and optionally substituted by 1-4 substituents each independently selected from:
—OH, —O—P(O)(OH)₂, —O—P(O)(R^{I}R^{II})₂, —OR^{c}, —NH₂, —NR^{c}R^{c}, —NR^{c}R^{d}, —CO₂H, —CO₂R^{c}, —OCOR^{c}, —CO₂H, —CO₂R^{c}, —SOR^{c}, —SO₂R^{c}, —CONH₂, —CONR^{c}R^{d}, —SO₂NH₂, —SO₂NR^{c}R^{d}, —OCONH₂, —OCONR^{c}R^{d}, —NR^{d}COR^{c}, —NR^{d}SOR^{c}, —NR^{d}CO₂R^{c}, —NR^{d}SO₂R^{c}, a fourth optionally substituted phenyl, a second optionally substituted 5-6 membered heterocycloalkyl, and a second optionally substituted 5-6 membered heteroaryl group, wherein:
the fourth optionally substituted phenyl, the second optionally substituted 5-6 membered heterocycloalkyl, and the second 5-6 membered heteroaryl are each independently and optionally substituted by 1-4 substituents each independently selected from:
a halogen, hydroxy, —O—P(O)(OH)₂, a —O—P(O)(R^{I}R^{II})₂, an amino, a (C₁-C₄alkyl)amino-, (C₁-C₄alkyl)(C₁-C₄alkyl)amino-, a C₁-C₄alkyl, halo(C₁-C₄alkyl), a hydroxy-(C₁-C₄alkyl)-, a —(C₁-C₄alkyl)-O—P(O)(OH)₂, a —(C₁-C₄alkyl)-O—P(O)(R¹R¹)₂, a halo(C₁-C₄alkoxy)-, a C₁-C₄alkoxy-, a hydroxy-(C₂-C₄alkoxy)-, a —(C₂-C₄alkoxy)-O—P(O)(OH)₂, a —(C₂-C₄alkoxy)-O—P(O)(RR)₂, a C₁-C₄alkoxy-(C₁-C₄alkoxy)-, —COR^{d}, —CON(R^{d})(R^{f}), and —CO₂R^{d};
R¹⁴ is C₁-C₄alkyl, which is optionally substituted by a substituent selected from —OR^{c}, —NR^{c}R^{d}, —CO₂R^{c}, —CONR^{c}R^{d}, —SO₂NR^{c}R^{d}, and —OCONR^{c}R^{d};
R¹⁶ is hydrogen, a halogen, or a C₁-C₄alkyl;
R¹⁵ and R¹⁷ are each independently hydrogen, a cyclopropyl, or a C₁-C₄alkyl;
R^{a} is a hydrogen, —R^{c}, —COR^{c}, —CO₂H, —CO₂R^{c}, —SOR^{c}, —SO₂R^{c}, —CONH₂, —CONR^{c}R^{d}, —SO₂NH₂, or —SO₂NR^{c}R^{d};
each R^{b} is independently a C₁-C₄alkyl, a halo(C₁-C₄alkyl), a —(C₁-C₄alkyl)-OH, a —(C₁-C₄alkyl)-O—P(O)(OH)₂, a —(C₁-C₄alkyl)-O—P(O)(R^{I}R^{II})₂, a —(C₁-C₄alkyl)-O—(C₁-C₄alkyl), a —(C₁-C₄alkyl)-N(R^{e})(R^{f}), a —(C₁-C₄alkyl)-O—CO(C₁-C₄alkyl), or a —(C₁-C₄alkyl)-CO—O—(C₁-C₄alkyl);
each R^{c} is independently a C₁-C₄alkyl, a halo(C₁-C₄alkyl), a —(C₁-C₄alkyl)-OH, a —(C₁-C₄alkyl)-O—P(O)(OH)₂, a —(C₁-C₄alkyl)-O—P(O)(R¹R¹)₂, a —(C₁-C₄alkyl)-O—(C₁-C₄alkyl), a —(C₁-C₄alkyl)-N(R^{e})(R^{f}), a —(C₁-C₄alkyl)-O—CO(C₁-C₄alkyl), a —(C₁-C₄alkyl)-CO—O—(C₁-C₄alkyl), a third optionally substituted C₃-C₆cycloalkyl, a fifth optionally substituted phenyl, a third optionally substituted 4-6 membered heterocycloalkyl, a third optionally substituted 5-6 membered heteroaryl, a second optionally substituted 9-10 membered heteroaryl, an optionally substituted —C₁-C₄alkyl-C₃-C₆cycloalkyl, a optionally substituted —C₁-C₄alkyl-phenyl, an optionally substituted —C₁-C₄alkyl-4-6 membered heterocycloalkyl, an optionally substituted —C₁-C₄alkyl-5-6 membered heteroaryl, or an optionally substituted —C₁-C₄alkyl-9-10 membered heteroaryl, wherein the third optionally substituted C₃-C₆cycloalkyl, the fifth optionally substituted phenyl, the third optionally substituted 4-6 membered heterocycloalkyl, the third optionally substituted 5-6 membered heteroaryl, the 9-10 membered heteroaryl moiety of the optionally substituted —C₁-C₄alkyl-9-10 membered heteroaryl, and the C₃-C₆cycloalkyl moiety of the optionally substituted —C₁-C₄alkyl-C₃-C₆cycloalkyl are each independently and optionally substituted by 1-4 substituents each independently selected from a halogen, hydroxy, —O—P(O)(OH)₂, —O—P(O)(R^{I}R^{II})₂, an amino, a —(C₁-C₄alkyl)NH₂, a (C₁-C₄alkyl)amino-, a (C₁-C₄alkyl)(C₁-C₄alkyl)amino-, a C₁-C₄alkyl, halo(C₁-C₄alkyl), a halo(C₁-C₄alkoxy)-, a C₁-C₄alkoxy-, hydroxy-(C₂-C₄alkoxy)-, a —(C₂-C₄alkoxy)-O—P(O)(OH)₂, a —(C₂-C₄alkoxy)-O—P(O)(R^{I}R^{II})₂, a C₁-C₄alkoxy-(C₁-C₄alkoxy)-, —COR^{d}, —CON(R^{d})(R^{f}), and —CO₂R^{d};
each R^{d} is independently H or a C₁-C₄alkyl;
each R^{c} is independently H, a (C₁-C₄alkyl), a —CO(C₁-C₄alkyl), a —OCO(C₁-C₄alkyl), a —CO₂(C₁-C₄alkyl), a —(C₁-C₄alkyl)NH₂, a —(C₁-C₄alkyl)C₁-C₄alkoxy, an optionally substituted —CO-(5-6 membered heterocycloalkyl), an optionally substituted —CO(C₁-C₄alkyl)-(5-6 membered heterocycloalkyl), an optionally substituted —CO(5-6 membered heteroaryl), an optionally substituted —CO(C₁-C₄alkyl)-(5-6 membered heteroaryl) wherein the 5-6 membered heterocycloalkyl moiety of the optionally substituted —CO-(5-6 membered heterocycloalkyl), the 5-6 membered heterocycloalkyl moiety of the optionally substituted —CO(C₁-C₄alkyl)-(5-6 membered heterocycloalkyl), the 5-6 membered heteroaryl moiety of the optionally substituted —CO(5-6 membered heteroaryl), and the 5-6 membered heteroaryl moiety of the optionally substituted —CO(C₁-C₄alkyl)-(5-6 membered heteroaryl) are each independently and optionally substituted 1-4 substituents each independently selected from:
a halogen, hydroxy, —O—P(O)(OH)₂, —O—P(O)(R^{I}R^{II})₂, an amino, a (C₁-C₄alkyl)amino-, a (C₁-C₄alkyl)(C₁-C₄alkyl)amino-, a C₁-C₄alkyl, halo(C₁-C₄alkyl), a halo(C₁-C₄alkoxy)-, a C₁-C₄alkoxy-, hydroxy-(C₂-C₄alkoxy)-, a —(C₂-C₄alkoxy)O—P(O)(OH)₂, a —(C₂-C₄alkoxy)-O—P(O)(R^{I}R^{II})₂, a C₁-C₄alkoxy-(C₁-C₄alkoxy)-, —COR^{d}, —CON(R^{d})(R^{f}), and —CO₂R^{d};
each R^{f} is independently hydrogen or a (C₁-C₄alkyl);
R^{g} and R^{h} are each independently hydrogen or a (C₁-C₄alkyl) or R^{g} and R^{h}, taken together with the atom or atoms through which they are connected, form a 5-6 membered ring; and
each occurrence of R^{I} and R^{II} are independently a (C₁-C₆alkyl)oxy.

36. The immunogenic composition according to claim 10, wherein the STING agonist is: 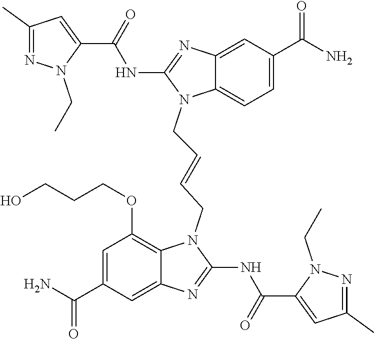
((E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-hydroxypropoxy)-1H-benzo[d]imidazole-5-carboxamide) 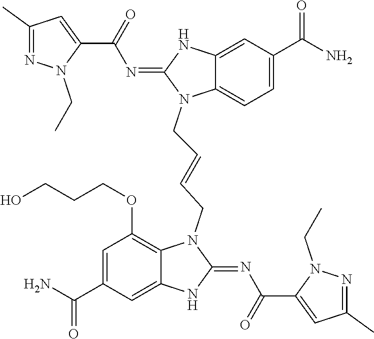
((E)-1-((E)-4-((E)-5-carbamoyl-2-((1-ethyl-3-methyl-1H-pyrazole-5-carbonyl)imino)-2,3-dihydro-1H-benzol[d]imidazol-1-yl)but-2-en-1-yl)-2-((1-ethyl-3-methyl-1H-pyrazole-5-carbonyl)imino)-7-(3-hydroxypropoxy)-2,3-dihydro-1H-benzo[d]imidazole-5-carboxamide), 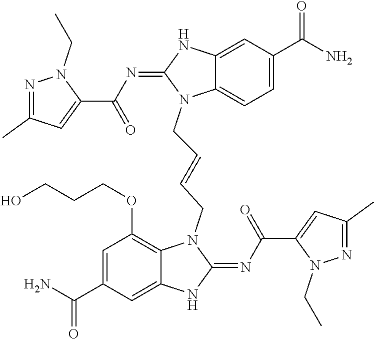
((Z)-1-((E)-4-((Z)-5-carbamoyl-2-((1-ethyl-3-methyl-1H-pyrazole-5-carbonyl)imino)-2,3-dihydro-1H-benzo[d]imidazol-1-yl)but-2-en-1-yl)-2-((1-ethyl-3-methyl-1H-pyrazole-5-carbonyl)imino)-7-(3-hydroxypropoxy)-2,3-dihydro-1H-benzo[d]imidazole-5-carboxamide); 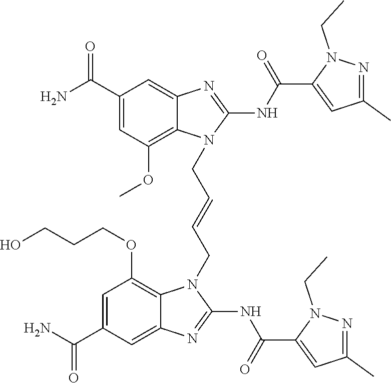 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCO)c21]
((E)-1-(4-(5-carbamoyl-2-(1-ethyl-3-methyl-1H-pyrazole-5-carboxamido)-7-(3-hydroxypropoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide)i 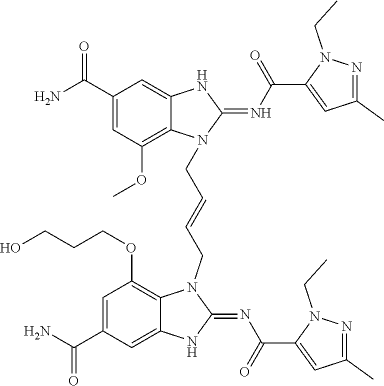 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCO)c21]
((E)-1-((E)-4-((E)-5-carbamoyl-2-((1-ethyl-3-methyl-1H-pyrazole-5-carbonyl)imino)-7-(3-hydroxypropoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide), 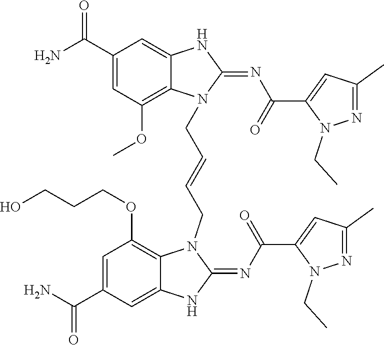 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCO)c21]
((Z)-1-((E)-4-((Z)-5-carbamoyl-2-((1-ethyl-3-methyl-1H-pyrazole-5-carbonyl)imino)-7-(3-hydroxypropoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide); 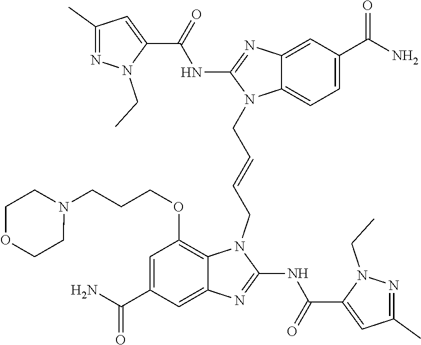 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCOCC3)c21]
((E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-morpholinopropoxy)-1H-benzo[d]imidazole-5-carboxamide). 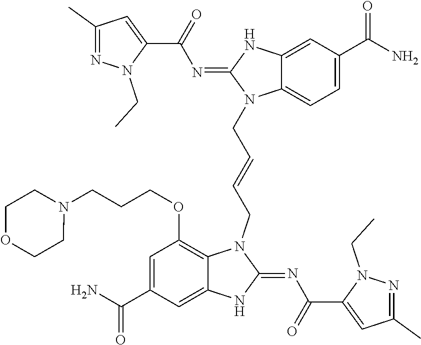 [SMILES: CCn1nc(C)cc1C(=O)/N=c1\[nH]c2cc(C(N)=O)ccc2n1C/C=C/Cn1/c(=N/C(=O)c2cc(C)nn2CC)[nH]c2cc(C(N)=O)cc(OCCCN3CCOCC3)c21]
((E)-1-((E)-4-((E)-5-carbamoyl-2-((1-ethyl-3-methyl-1H-pyrazole-5-carbonyl)imino)-2,3-dihydro-1H-benzo[d]imidazol-1-yl)but-2-en-1-yl)-2-((1-ethyl-3-methyl-1H-pyrazole-5-carbonyl)imino)-7-(3-morpholinopropoxy)-2,3-dihydro-1H-benzo[d]imidazole-5-carboxamide), 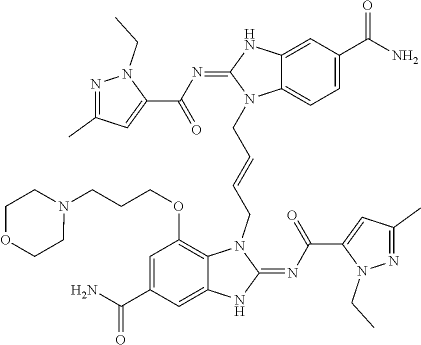 [SMILES: CCn1nc(C)cc1C(=O)/N=c1/[nH]c2cc(C(N)=O)ccc2n1C/C=C/Cn1/c(=N\C(=O)c2cc(C)nn2CC)[nH]c2cc(C(N)=O)cc(OCCCN3CCOCC3)c21]
((Z)-1-((E)-4-((Z)-5-carbamoyl-2-((1-ethyl-3-methyl-1H-pyrazole-5-carbonyl)imino)-2,3-dihydro-1H-benzol[d]imidazol-1-yl)but-2-en-1-yl)-2-((1-ethyl-3-methyl-1H-pyrazole-5-carbonyl)imino)-7-(3-morpholinopropoxy)-2,3-dihydro-1H-benzo[d]imidazole-5-carboxamide); 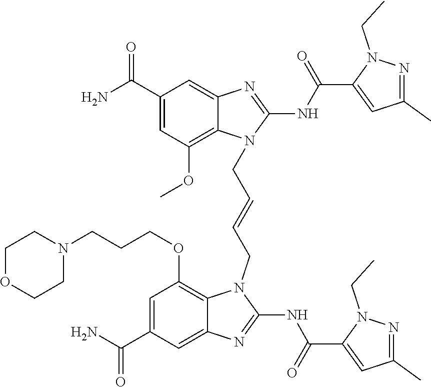 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCOCC3)c21]
((E)-1-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide); 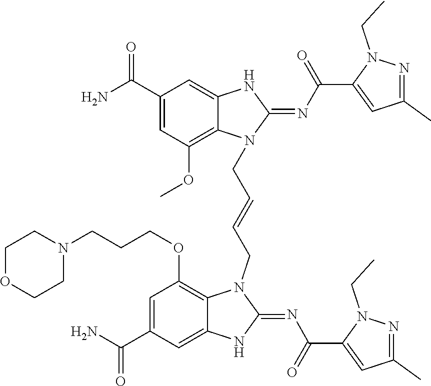 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCOCC3)c21]
(E)-1-((E)-4-((E)-5-carbamoyl-2-((1-ethyl-3-methyl-1H-pyrazole-5-carbonyl)imino)-7-(3-morpholinopropoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide), 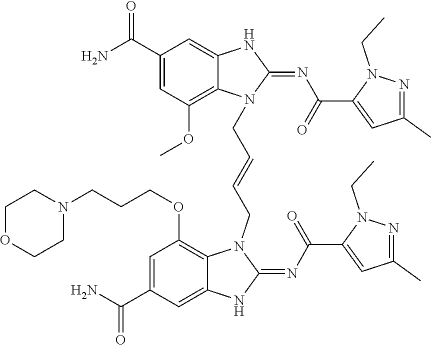 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCOCC3)c21]
((Z)-1-((E)-4-((Z)-5-carbamoyl-2-((1-ethyl-3-methyl-1H-pyrazole-5-carbonyl)imino)-7-(3-morpholinopropoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide); 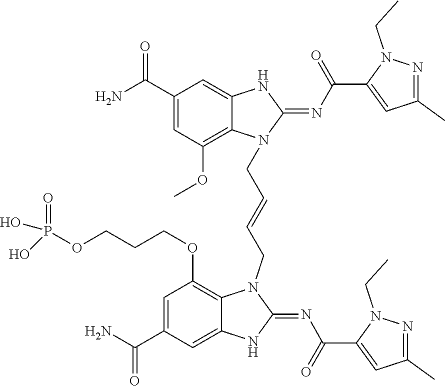 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCOP(=O)(O)O)c21]
(3-(((Z)-6-carbamoyl-3-((E)-4-((Z)-5-carbamoyl-2-((1-ethyl-3-methyl-1H-pyrazole-5-carbonyl)imino)-7-methoxy-2,3-dihydro-1H-benzo[d]imidazol-1-yl)but-2-en-1-yl)-2-((1-ethyl-3-methyl-1H-pyrazole-5-carbonyl)imino)-2,3-dihydro-1H-benzo[d]imidazol-4-yl)oxy)propyldihydrogen phosphate). 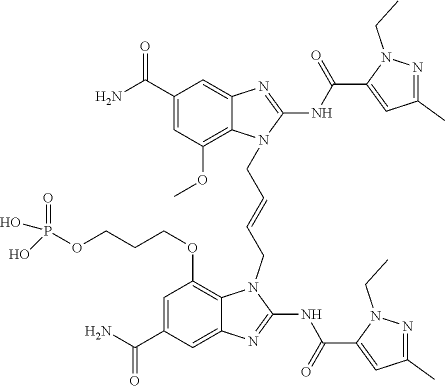 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCOP(=O)(O)O)c21]
(E3-((5-carbamoyl-1-((4-(5-carbamoyl-2-((1-ethyl-3-methyl-H-pyrazole-5-carboxamido)-7-methoxy-1H-benzo[d]imidazol-yl)but-2-en-1-yl)-2-(1-ethyl-3-methyl-1H-pyrazole-5-carboxamido)-1H-benzo[d]imidazol-7-yl)oxy)propyl dihydrogen phosphate); 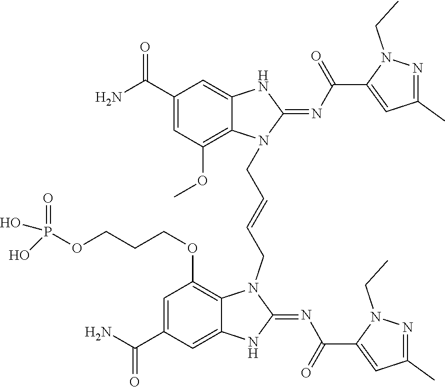 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCOP(=O)(O)O)c21]
(3-(((E)-6-carbamoyl-3-((E)-4-((E)-5-carbamoyl-2-((1-ethyl-3-methyl-1H-pyrazole-5-carbonyl)imino)-7-methoxy-2,3-dihydro-TH-benzo[d]imidazol-1-yl)but-2-en-1-yl)-2-((1-ethyl-3-methyl-1H-pyrazole-5-carbonyl)imino)-2,3-dihydro-1H-benzo[d]imidazol-4-yl)oxy)propyl dihydrogen phosphate)f 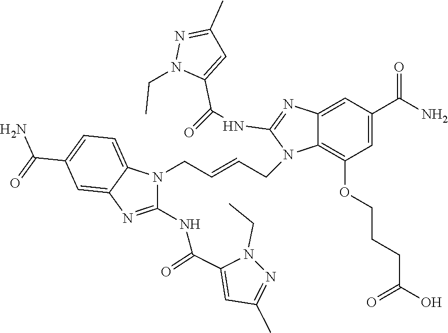 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCC(=O)O)c21]
((E)-4-((5-carbamoyl-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)butanoic acid); 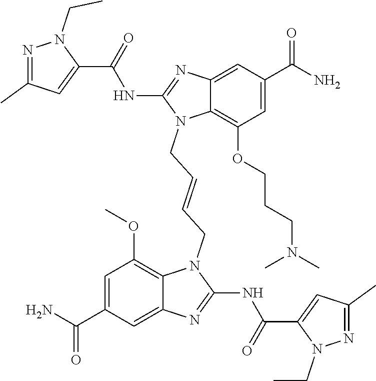
((E)-1-(4-(5-carbamoyl-2-(1-ethyl-3-methyl-1H-pyrazole-5-carboxamido)-7-methoxy-1H-benzo[d]imidazol-1-yl)but-2-en-1-yl)-7-(3-(dimethylamino)propoxy)-2-(1-ethyl-3-methyl-1H-pyrazole-5-carboxamido)-1H-benzo[d]imidazole-5-carboxamide) 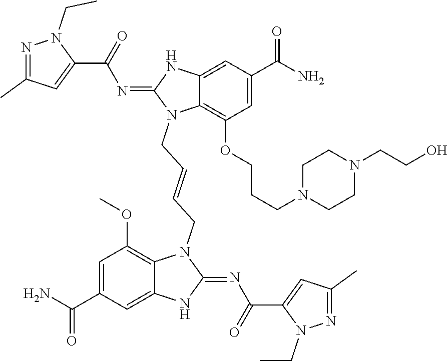 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(CCO)CC3)c21]
((E)-1-((E)-4-((E)-5-carbamoyl-2-((1-ethyl-3-methyl-1H-pyrazole-5-carbonyl)imino)-7-(3-(4-(2-hydroxyethyl)piperazin-1-yl)propoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide);
a pharmaceutically acceptable salt thereof; a tautomer thereof; or any combination thereof.

37. The immunogenic composition of claim 17, wherein the STING agonist is: 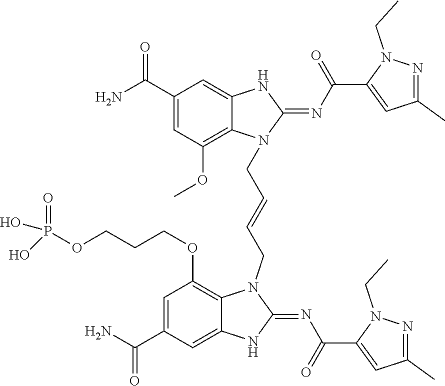 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCOP(=O)(O)O)c21]
(3-(((E)-6-carbamoyl-3-((E)-4-((E)-5-carbamoyl-2-((1-ethyl-3-methyl-1H-pyrazole-5-carbonyl)imino)-7-methoxy-2,3-dihydro-1H-benzo[d]imidazol-1-yl)but-2-en-1-yl)-2-((l-ethyl-3-methyl-1H-pyrazole-5-carbonyl)imino)-2,3-dihydro-1H-benzo[d]imidazol-4-yl)oxy)propyl dihydrogen phosphate), a pharmaceutically acceptable salt thereof, a tautomer thereof, or any combination thereof.

38.-203. (canceled)


SEQUENCE LISTING (13 sequences)

SEQ ID NO: 1 - protein, 1273 aa, Human coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1208 aa, Human coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 3 - protein, 192 aa, Human coronavirus
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 4 - protein, 1208 aa, Artificial Sequence
  SARS-CoV-2 Stabilized Spike protein ectodomain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 5 - DNA, 2864 bp, Artificial Sequence
  Polynucleotide sequence of HSV-2 gE/gI insert
gggcccctataactctctacggctaacctgaatggactacgacatagtctagtccgccaagatggccaggggagccggcctcgtgttcttcgtgggcgtgtgggtggtgagctgtctggccgctgcccctagaaccagctggaagagagtgacctccggagaggacgtggtgctgctccctgctcccgctggacctgaggaaagaaccagagcccacaagctgctgtgggctgctgagcctctggacgcctgcggacctctgagaccttcctgggtggctctgtggcctcccagaagagtgctggagacagtggtggacgccgcctgcatgagagcccctgaacccctggccatcgcctactcccccccttttcccgccggagacgagggcctgtatagcgagctggcctggagagacagggtggccgtggtcaatgagagcctggtcatctacggagccctggagaccgacagcggcctgtataccctgagcgtggtgggcctgagcgacgaagccagacaggtggcttccgtcgtgctggtggtggaacctgcccccgtccctacacctacccctgacgactacgacgaggaggacgacgccggcgtgtccgaaagaacccccgtgagcgtgcctccccccacccctcctagaagacctcctgtggctcctcctacccaccccagggtgatccctgaggtgagccacgtcagaggcgtgaccgtgcacatggagacacctgaggccatcctgttcgcccctggcgagaccttcggaaccaatgtgtccatccacgccattgcccacgacgacggcccctacgccatggacgtggtgtggatgaggtttgacgtgccctccagctgcgccgagatgaggatctacgaggcctgcctgtaccacccccagctgcccgagtgtctgtcccctgctgacgctccttgcgccgtcagcagctgggcctatagactggccgtgagatcctacgctggctgtagcagaaccaccaggccccccagatgcttcgccgaagccagaatggagcctgtgcctggactggcctggctggcttccaccgtgaatctggagtttcagcacgccagcccccaacacgccggactgtacctgtgcgtggtgtacgtggacgaccacatccatgcctggggacacatgaccatcagcaccgccgcccagtacaggaacgctgtggtggaacagcacctgccccagaggcaacctgagcccgtggagcccaccagacctcatgtgagagcccctccccctgctccctccgccagaggacctctgagactgggctgattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagtctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcctggcaggagcctgcaaggcctggccatcctgggcctgtgggtctgtgctaccggcctggtggtgagaggacccaccgtgtccctggtgagcgacagcctggtggatgccggagctgtgggacctcagggcttcgtggaagaggacctgagggtgttcggcgaactccactttgtgggcgcccaggtgccccacaccaactactacgacggcatcatcgagctgttccactaccccctgggtaatcattgtcccagagtggtgcacgtggtgaccctgaccgcctgtcctagaaggcccgctgtggccttcaccctgtgcagaagcacccaccacgcccacagccctgcctaccccacactggaactgggactggccagacagcccctgctgagggtgaggacagccaccagggactatgccggactgtacgtgctgagagtgtgggtgggaagcgccaccaacgcctccctgttcgtgctgggcgtggctctgagcgccaatggcaccttcgtgtacaacggctccgactatggcagctgtgaccccgcccagctcccctttagcgctcctaggctgggacctagctccgtgtacacccccggagccagcagacccacaccccccagaaccaccaccagccctagctcccctcgtgatcctacccctgcccctggcgatacaggaacccctgcccccgctagcggagaaagagcccctcccaacagcaccaggtccgccagcgagtccaggcacaggctgacatgatgaggcgcgcccacccagcggccgc

SEQ ID NO: 6 - DNA, 12775 bp, Artificial Sequence
  Polynucleotide sequence of parent SAM pTC83R_P989 plasmid
aagttgggtaacgccagggttttcccagtcacacgcgtaatacgactcactatagataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggccaggggagccggcctcgtgttcttcgtgggcgtgtgggtggtgagctgtctggccgctgcccctagaaccagctggaagagagtgacctccggagaggacgtggtgctgctccctgctcccgctggacctgaggaaagaaccagagcccacaagctgctgtgggctgctgagcctctggacgcctgcggacctctgagaccttcctgggtggctctgtggcctcccagaagagtgctggagacagtggtggacgccgcctgcatgagagcccctgaacccctggccatcgcctactcccccccttttcccgccggagacgagggcctgtatagcgagctggcctggagagacagggtggccgtggtcaatgagagcctggtcatctacggagccctggagaccgacagcggcctgtataccctgagcgtggtgggcctgagcgacgaagccagacaggtggcttccgtcgtgctggtggtggaacctgcccccgtccctacacctacccctgacgactacgacgaggaggacgacgccggcgtgtccgaaagaacccccgtgagcgtgcctccccccacccctcctagaagacctcctgtggctcctcctacccaccccagggtgatccctgaggtgagccacgtcagaggcgtgaccgtgcacatggagacacctgaggccatcctgttcgcccctggcgagaccttcggaaccaatgtgtccatccacgccattgcccacgacgacggcccctacgccatggacgtggtgtggatgaggtttgacgtgccctccagctgcgccgagatgaggatctacgaggcctgcctgtaccacccccagctgcccgagtgtctgtcccctgctgacgctccttgcgccgtcagcagctgggcctatagactggccgtgagatcctacgctggctgtagcagaaccaccaggccccccagatgcttcgccgaagccagaatggagcctgtgcctggactggcctggctggcttccaccgtgaatctggagtttcagcacgccagcccccaacacgccggactgtacctgtgcgtggtgtacgtggacgaccacatccatgcctggggacacatgaccatcagcaccgccgcccagtacaggaacgctgtggtggaacagcacctgccccagaggcaacctgagcccgtggagcccaccagacctcatgtgagagcccctccccctgctccctccgccagaggacctctgagactgggctgattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagtctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcctggcaggagcctgcaaggcctggccatcctgggcctgtgggtctgtgctaccggcctggtggtgagaggacccaccgtgtccctggtgagcgacagcctggtggatgccggagctgtgggacctcagggcttcgtggaagaggacctgagggtgttcggcgaactccactttgtgggcgcccaggtgccccacaccaactactacgacggcatcatcgagctgttccactaccccctgggtaatcattgtcccagagtggtgcacgtggtgaccctgaccgcctgtcctagaaggcccgctgtggccttcaccctgtgcagaagcacccaccacgcccacagccctgcctaccccacactggaactgggactggccagacagcccctgctgagggtgaggacagccaccagggactatgccggactgtacgtgctgagagtgtgggtgggaagcgccaccaacgcctccctgttcgtgctgggcgtggctctgagcgccaatggcaccttcgtgtacaacggctccgactatggcagctgtgaccccgcccagctcccctttagcgctcctaggctgggacctagctccgtgtacacccccggagccagcagacccacaccccccagaaccaccaccagccctagctcccctcgtgatcctacccctgcccctggcgatacaggaacccctgcccccgctagcggagaaagagcccctcccaacagcaccaggtccgccagcgagtccaggcacaggctgacatgatgaggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgatt

SEQ ID NO: 7 - DNA, 10522 bp, Artificial Sequence
  Polynucleotide sequence of SAM gE_P317R_IRES_gI transcript
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggccaggggagccggcctcgtgttcttcgtgggcgtgtgggtggtgagctgtctggccgctgcccctagaaccagctggaagagagtgacctccggagaggacgtggtgctgctccctgctcccgctggacctgaggaaagaaccagagcccacaagctgctgtgggctgctgagcctctggacgcctgcggacctctgagaccttcctgggtggctctgtggcctcccagaagagtgctggagacagtggtggacgccgcctgcatgagagcccctgaacccctggccatcgcctactcccccccttttcccgccggagacgagggcctgtatagcgagctggcctggagagacagggtggccgtggtcaatgagagcctggtcatctacggagccctggagaccgacagcggcctgtataccctgagcgtggtgggcctgagcgacgaagccagacaggtggcttccgtcgtgctggtggtggaacctgcccccgtccctacacctacccctgacgactacgacgaggaggacgacgccggcgtgtccgaaagaacccccgtgagcgtgcctccccccacccctcctagaagacctcctgtggctcctcctacccaccccagggtgatccctgaggtgagccacgtcagaggcgtgaccgtgcacatggagacacctgaggccatcctgttcgcccctggcgagaccttcggaaccaatgtgtccatccacgccattgcccacgacgacggcccctacgccatggacgtggtgtggatgaggtttgacgtgccctccagctgcgccgagatgaggatctacgaggcctgcctgtaccacccccagctgcccgagtgtctgtcccctgctgacgctccttgcgccgtcagcagctgggcctatagactggccgtgagatcctacgctggctgtagcagaaccaccaggccccccagatgcttcgccgaagccagaatggagcctgtgcctggactggcctggctggcttccaccgtgaatctggagtttcagcacgccagcccccaacacgccggactgtacctgtgcgtggtgtacgtggacgaccacatccatgcctggggacacatgaccatcagcaccgccgcccagtacaggaacgctgtggtggaacagcacctgccccagaggcaacctgagcccgtggagcccaccagacctcatgtgagagcccctccccctgctccctccgccagaggacctctgagactgggctgattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagtctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgaccatgcctggcaggagcctgcaaggcctggccatcctgggcctgtgggtctgtgctaccggcctggtggtgagaggacccaccgtgtccctggtgagcgacagcctggtggatgccggagctgtgggacctcagggcttcgtggaagaggacctgagggtgttcggcgaactccactttgtgggcgcccaggtgccccacaccaactactacgacggcatcatcgagctgttccactaccccctgggtaatcattgtcccagagtggtgcacgtggtgaccctgaccgcctgtcctagaaggcccgctgtggccttcaccctgtgcagaagcacccaccacgcccacagccctgcctaccccacactggaactgggactggccagacagcccctgctgagggtgaggacagccaccagggactatgccggactgtacgtgctgagagtgtgggtgggaagcgccaccaacgcctccctgttcgtgctgggcgtggctctgagcgccaatggcaccttcgtgtacaacggctccgactatggcagctgtgaccccgcccagctcccctttagcgctcctaggctgggacctagctccgtgtacacccccggagccagcagacccacaccccccagaaccaccaccagccctagctcccctcgtgatcctacccctgcccctggcgatacaggaacccctgcccccgctagcggagaaagagcccctcccaacagcaccaggtccgccagcgagtccaggcacaggctgacatgatgaggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaa

SEQ ID NO: 8 - RNA, 10522 nt, Artificial Sequence
  SAM pTC83R_P989 transcript theoretical RNA sequence
gauaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagauggccaggggagccggccucguguucuucgugggcguguggguggugagcugucuggccgcugccccuagaaccagcuggaagagagugaccuccggagaggacguggugcugcucccugcucccgcuggaccugaggaaagaaccagagcccacaagcugcugugggcugcugagccucuggacgccugcggaccucugagaccuuccuggguggcucuguggccucccagaagagugcuggagacagugguggacgccgccugcaugagagccccugaaccccuggccaucgccuacucccccccuuuucccgccggagacgagggccuguauagcgagcuggccuggagagacaggguggccguggucaaugagagccuggucaucuacggagcccuggagaccgacagcggccuguauacccugagcguggugggccugagcgacgaagccagacagguggcuuccgucgugcuggugguggaaccugcccccgucccuacaccuaccccugacgacuacgacgaggaggacgacgccggcguguccgaaagaacccccgugagcgugccuccccccaccccuccuagaagaccuccuguggcuccuccuacccaccccagggugaucccugaggugagccacgucagaggcgugaccgugcacauggagacaccugaggccauccuguucgccccuggcgagaccuucggaaccaauguguccauccacgccauugcccacgacgacggccccuacgccauggacgugguguggaugagguuugacgugcccuccagcugcgccgagaugaggaucuacgaggccugccuguaccacccccagcugcccgagugucuguccccugcugacgcuccuugcgccgucagcagcugggccuauagacuggccgugagauccuacgcuggcuguagcagaaccaccaggccccccagaugcuucgccgaagccagaauggagccugugccuggacuggccuggcuggcuuccaccgugaaucuggaguuucagcacgccagcccccaacacgccggacuguaccugugcgugguguacguggacgaccacauccaugccuggggacacaugaccaucagcaccgccgcccaguacaggaacgcugugguggaacagcaccugccccagaggcaaccugagcccguggagcccaccagaccucaugugagagccccucccccugcucccuccgccagaggaccucugagacugggcugauuaaaacagcuguggguuguucccacccacagggcccacugggcgcuagcacucugauuuuacgaaauccuugugcgccuguuuuauaucccuucccuaauucgaaacguagaagcaaugcgcaccacugaucaauaguaggcguaacgcgccaguuacgucaugaucaagcauaucuguucccccggacugaguaucaauagacugcuuacgcgguugaaggagaaaacguucguuauccggcuaacuacuucgagaagcccaguaacaccauggaagcugcaggguguuucgcucagcacuucccccguguagaucaggucgaugagccacugcaauccccacaggugacuguggcaguggcugcguuggcggccugccuauggggagacccauaggacgcucuaauguggacauggugcgaagagucuauugagcuaguuaguaguccuccggccccugaaugcggcuaauccuaacugcggagcacaugccuucaacccagaggguagugugucguaacgggcaacucugcagcggaaccgacuacuuuggguguccguguuucuuuuuuauucuuauauuggcugcuuauggugacaauuacagaauuguuaccauauagcuauuggauuggccauccgguguguaauagagcuguuauauaccuauuuguuggcuuuguaccacuaacuuuaaaaucuauaacuacccucaacuuuauauuaacccucaauacaguugaccaugccuggcaggagccugcaaggccuggccauccugggccugugggucugugcuaccggccugguggugagaggacccaccgugucccuggugagcgacagccugguggaugccggagcugugggaccucagggcuucguggaagaggaccugaggguguucggcgaacuccacuuugugggcgcccaggugccccacaccaacuacuacgacggcaucaucgagcuguuccacuacccccuggguaaucauugucccagaguggugcacguggugacccugaccgccuguccuagaaggcccgcuguggccuucacccugugcagaagcacccaccacgcccacagcccugccuaccccacacuggaacugggacuggccagacagccccugcugagggugaggacagccaccagggacuaugccggacuguacgugcugagagugugggugggaagcgccaccaacgccucccuguucgugcugggcguggcucugagcgccaauggcaccuucguguacaacggcuccgacuauggcagcugugaccccgcccagcuccccuuuagcgcuccuaggcugggaccuagcuccguguacacccccggagccagcagacccacaccccccagaaccaccaccagcccuagcuccccucgugauccuaccccugccccuggcgauacaggaaccccugcccccgcuagcggagaaagagccccucccaacagcaccagguccgccagcgaguccaggcacaggcugacaugaugaggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaaa

SEQ ID NO: 9 - protein, 419 aa, Artificial Sequence
  Polypeptide sequence of HSV-2 gE P317R
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTRPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLG

SEQ ID NO: 10 - protein, 256 aa, Artificial Sequence
  Polypeptide sequence of HSV-2 gI
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTSPSSPRDPTPAPGDTGTPAPASGERAPPNSTRSASESRHRLT

SEQ ID NO: 11 - RNA, 1593 nt, Artificial Sequence
  gE mRNA with 5′ UTR, 3′ UTR and polyA tail
gggaaauaagagagaaaagaagaguaagaagaaauauaagaccccggcgccgccaccauggccaggggagccggccucguguucuucgugggcguguggguggugagcugucuggccgcugccccuagaaccagcuggaagagagugaccuccggagaggacguggugcugcucccugcucccgcuggaccugaggaaagaaccagagcccacaagcugcugugggcugcugagccucuggacgccugcggaccucugagaccuuccuggguggcucuguggccucccagaagagugcuggagacagugguggacgccgccugcaugagagccccugaaccccuggccaucgccuacucccccccuuuucccgccggagacgagggccuguauagcgagcuggccuggagagacaggguggccguggucaaugagagccuggucaucuacggagcccuggagaccgacagcggccuguauacccugagcguggugggccugagcgacgaagccagacagguggcuuccgucgugcuggugguggaaccugcccccgucccuacaccuaccccugacgacuacgacgaggaggacgacgccggcguguccgaaagaacccccgugagcgugccuccccccaccccuccuagaagaccuccuguggcuccuccuacccaccccagggugaucccugaggugagccacgucagaggcgugaccgugcacauggagacaccugaggccauccuguucgccccuggcgagaccuucggaaccaauguguccauccacgccauugcccacgacgacggccccuacgccauggacgugguguggaugagguuugacgugcccuccagcugcgccgagaugaggaucuacgaggccugccuguaccacccccagcugcccgagugucuguccccugcugacgcuccuugcgccgucagcagcugggccuauagacuggccgugagauccuacgcuggcuguagcagaaccaccccuccccccagaugcuucgccgaagccagaauggagccugugccuggacuggccuggcuggcuuccaccgugaaucuggaguuucagcacgccagcccccaacacgccggacuguaccugugcgugguguacguggacgaccacauccaugccuggggacacaugaccaucagcaccgccgcccaguacaggaacgcugugguggaacagcaccugccccagaggcaaccugagcccguggagcccaccagaccucaugugagagccccucccccugcucccuccgccagaggaccucugagacugggcugaucuagacggcgcgcccacccagcggccgcugauaauaggcuggagccucgguggccaugcuucuugccccuugggccuccccccagccccuccuccccuuccugcacccguacccccguggucuuugaauaaagucugauuuucauugcaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - RNA, 1593 nt, Artificial Sequence; identical to SEQ ID NO: 11
  gE mRNA with 5′ UTR, 3′ UTR and polyA tail - all U replaced with N1methyl-pseudouridine

SEQ ID NO: 13 - protein, 419 aa, Artificial Sequence
  HSV-2 gE
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLG